(19) United States
(12) Patent Application Publication
(10) Pub. No.: US 2020/0181226 A1
(43) Pub. Date: Jun. 11, 2020

(54) ANTIBODY-COUPLED T CELL RECEPTOR CONSTRUCTS AND THERAPEUTIC USES THEREOF

(71) Applicant: UNUM THERAPEUTICS INC., Cambridge, MA (US)

(72) Inventors: Kathleen MCGINNESS, Cambridge, MA (US); Gregory MOTZ, Cambridge, MA (US); Michael FRAY, Cambridge, MA (US); Birgit SCHULTES, Cambridge, MA (US); James HEMPHILL, Cambridge, MA (US); James KIM, Cambridge, MA (US); Tooba CHEEMA, Cambridge, MA (US); Taylor HICKMAN, Cambridge, MA (US)

(73) Assignee: UNUM THERAPEUTICS INC., Cambridge, MA (US)

(21) Appl. No.: 16/481,786

(22) PCT Filed: Jan. 30, 2018
(86) PCT No.: PCT/US2018/015999

§ 371 Date: Jul. 29, 2019

Related U.S. Application Data
(60) Provisional application No. 62/451,992, filed on Jan. 30, 2017.
(60) Provisional application No. 62/578,429, filed on Oct. 28, 2017.

(51) Int. Cl. C07K 14/725; C07K 16/28; A61K 39/395; C07K 14/705; C07K 14/735; A61P 35/00; C12N 5/0783; A61K 45/06

(52) U.S. Cl. CPC C07K 14/7051; C07K 16/2887; A61K 39/3955; C07K 14/70521; C07K 14/70535; C07K 14/70517; A61K 2039/505; C12N 5/0636; C12N 5/0646; A61K 45/06; C07K 2319/03; C07K 2319/02; A61P 35/00

(57) ABSTRACT

Disclosed herein are antibody-coupled T cell receptor (ACTR) polypeptides comprising: a CD16A extracellular domain, a transmembrane domain, one or more co-stimulatory signaling domains, at least one of which is a CD28 co-stimulatory signaling domain, and a CD3z cytoplasmic signaling domain. Also disclosed herein are genetically engineered immune cells, expressing: a first polypeptide which is an antibody-coupled T cell receptor (ACTR); and a second polypeptide that elicits a co-stimulatory signal as well as methods of enhancing antibody-dependent cell cytotoxicity (ADCC) in a subject comprising administering to a subject in need thereof a therapeutically effective amount of a therapeutic antibody and an effective amount of immune cells (e.g., T lymphocytes and/or NK cells) expressing an antibody-coupled T-cell receptor (ACTR) polypeptide.

### CROSS REFERENCE TO RELATED APPLICATIONS

This application claims the benefit of the filing date of U.S. Provisional Application No. 62/451,992, filed Jan. 30, 2017, and U.S. Provisional Application No. 62/578,429, filed Oct. 28, 2017. The entire contents of each of these referenced applications are incorporated by reference herein.

### BACKGROUND OF DISCLOSURE

Cancer immunotherapy, including cell-based therapy, antibody therapy and cytokine therapy, is used to provoke immune responses attacking tumor cells while sparing normal tissues. It is a promising option for treating various types of cancer because of its potential to evade genetic and cellular mechanisms of drug resistance, and to target tumor cells while sparing normal tissues. T-lymphocytes can exert major anti-tumor effects as demonstrated by results of allogeneic hematopoietic stem cell transplantation (HSCT) for hematologic malignancies, where T-cell-mediated graft-versus-host disease (GvHD) is inversely associated with disease recurrence, and immunosuppression withdrawal or infusion of donor lymphocytes can contain relapse. Weiden et al., N Engl J Med. 1979; 300(19):1068-1073; Porter et al., N Engl J Med. 1994; 330(2):100-106; Kolb et al., Blood. 1995; 86(5):2041-2050; Slavin et al., Blood. 1996; 87(6):2195-2204; and Appelbaum, Nature. 2001; 411(6835):385-389.

Cell-based therapy may involve cytotoxic T cells having reactivity skewed toward cancer cells. Eshhar et al., Proc. Natl. Acad. Sci. U.S.A; 1993; 90(2):720-724; Geiger et al., J Immunol. 1999; 162(10):5931-5939; Brentjens et al., Nat. Med. 2003; 9(3):279-286; Cooper et al., Blood. 2003; 101(4):1637-1644; and Imai et al., Leukemia. 2004; 18:676-684. One approach is to express a chimeric receptor having an antigen-binding domain fused to one or more T cell activation signaling domains. Binding of a cancer antigen via the antigen-binding domain results in T cell activation and triggers cytotoxicity. Recent results of clinical trials with infusions of chimeric receptor-expressing autologous T lymphocytes provided compelling evidence of their clinical potential. Pule et al., Nat. Med. 2008; 14(11):1264-1270; Porter et al., N Engl J Med; 2011; 25; 365(8):725-733; Brentjens et al., Blood. 2011; 118(18):4817-4828; Till et al., Blood. 2012; 119(17):3940-3950; Kochenderfer et al., Blood. 2012; 119(12):2709-2720; and Brentjens et al., Sci Transl Med. 2013; 5(177):177ra138.

Another approach is to express an antibody-coupled T cell Receptor (ACTR) protein in an immune cell, such as an NK cell or a T cell, the ACTR protein containing an extracellular Fc-binding domain. When the ACTR-expressing T cells (also called “ACTR T cells”) are administered to a subject together with an anti-cancer antibody, they may enhance toxicity against cancer cells targeted by the antibody via their binding to the Fc domain of the antibody. Kudo et al., Cancer Research (2014) 74:93-103.

Antibody-based immunotherapies, such as monoclonal antibodies, antibody-fusion proteins, and antibody drug conjugates (ADCs) are used to treat a wide variety of diseases, including many types of cancer. Such therapies may depend on recognition of cell surface molecules that are differentially expressed on cells for which elimination is desired (e.g., target cells such as cancer cells) relative to normal cells (e.g., non-cancer cells). Binding of an antibody-based immunotherapy to a cancer cell can lead to cancer cell death via various mechanisms, e.g., antibody-dependent cell-mediated cytotoxicity (ADCC), complement-dependent cytotoxicity (CDC), or direct cytotoxic activity of the payload from an antibody-drug conjugate (ADC).

### SUMMARY OF DISCLOSURE

The present disclosure is based on the development of improved antibody-coupled T-cell receptors (ACTRs), which exhibited superior in vitro and/or in vivo bioactivities, including therapeutic activities. Such improved ACTR constructs may contain a CD28 co-stimulatory domain. Alternatively or in addition, the improved ACTR constructs described herein may have no or a shortened hinge domain. T cells expressing the improved ACTR constructs described herein, either taken alone, or in combination with a separate polypeptide capable of eliciting a co-stimulatory signaling exhibited superior in vivo and in vitro bioactivities, including cytotoxicity, cell proliferation and activation (e.g., IL-2 production, percentage of CD3⁺ cells), and/or in vivo anti-tumor activity.

Accordingly, provided herein are ACTR polypeptides having one or more enhanced bioactivity, nucleic acids encoding such, immune cells (e.g., T cells or natural killing cells) expressing the ACTR, and optionally a separately polypeptide capable of eliciting a co-stimulatory signaling, and uses of such, together with a therapeutic antibody, in immune therapies.

Accordingly, one aspect of the present disclosure features an antibody-coupled T cell receptor (ACTR) polypeptide, comprising: (i) a CD16A extracellular domain, (ii) a transmembrane domain, (iii) one or more co-stimulatory signaling domains, at least one of which is a CD28 co-stimulatory signaling domain, and (iv) a CD3ζ cytoplasmic signaling domain.

Another aspect of the present disclosure features an antibody-coupled T cell receptor (ACTR) polypeptide, comprising: (i) a CD16A extracellular domain, (ii) a transmembrane domain, (iii) one or more co-stimulatory signaling domains, at least one of which is a CD28 co-stimulatory signaling domain, and (iv) a CD3ζ cytoplasmic signaling domain. If the transmembrane domain (ii) is a CD8 transmembrane domain, the ACTR polypeptide is either free of a hinge domain from any non-CD16A receptor, or comprises more than one co-stimulatory signaling domains.

In some embodiments, the ACTR polypeptide further comprises a hinge domain, which may be 1 to 60 amino acid residues in length (e.g., 1 to 30 amino acid residues in length or 31 to 60 amino acid residues in length). In other embodiments, the ACTR polypeptide described herein may be free of any hinge domain from a non-CD16A receptor. In some examples, the ACTR polypeptide may be free of any hinge domain.

In some embodiments, the hinge domain is a CD16A hinge domain, a non-CD16A receptor hinge domain, or a combination thereof. In certain embodiments, the hinge domain comprises a CD28 hinge domain.

In some embodiments, the transmembrane domain (ii) is a CD28 transmembrane domain. In that case, the ACTR polypeptide may be free of any hinge domain from any non-CD16A receptor and/or comprises more than one co-stimulatory domains.

In some embodiments, the ACTR polypeptide comprises (i) the CD28 co-stimulatory domain; and (ii) a CD28 transmembrane domain, a CD28 hinge domain, or a combination thereof.

In some examples, the ACTR polypeptide comprises the amino acid sequence of SEQ ID NO: 9, SEQ ID NO: 19, SEQ ID NO: 20, SEQ ID NO: 21, SEQ ID NO: 22, or SEQ ID NO: 27.

In some embodiments, the ACTR polypeptide comprises two co-stimulatory signaling domains, one being a CD28 co-stimulatory signaling domain and the other being a 4-1BB co-stimulatory signaling domain or an OX40 co-stimulatory signaling domain. In some embodiments, the other co-stimulatory signaling domain is a 4-1BB co-stimulatory signaling domain. In certain embodiments, the 4-1BB signaling domain is located N-terminal to the CD28 co-stimulatory signaling domain. In certain embodiments, the 4-1BB signaling domain is located C-terminal to the CD28 co-stimulatory signaling domain. In some embodiments, the other co-stimulatory signaling domain is an OX40 co-stimulatory signaling domain. In certain embodiments, the OX40 co-stimulatory signaling domain is located C-terminal to the CD28 co-stimulatory signaling domain. In certain embodiments, the OX40 co-stimulatory signaling domain is located N-terminal to the CD28 co-stimulatory signaling domain.

In some embodiments, the transmembrane domain (ii) is a CD8 transmembrane domain.

In some embodiments, any of the ACTR polypeptides described herein may further comprise a CD8 hinge domain. In certain examples, the ACTR polypeptide comprises an amino acid sequence of SEQ ID NO: 7 or SEQ ID NO: 8.

In one aspect, the present disclosure provides an antibody-coupled T cell receptor (ACTR) polypeptide, comprising: (i) a CD16A extracellular domain, (ii) a transmembrane domain, and (iii) a CD3ζ cytoplasmic signaling domain. Such an ACTR polypeptide may be free of a hinge domain from any non-CD16A receptor (e.g., is free of any hinge domain).

In some embodiments, ACTR polypeptide further comprises one or more co-stimulatory signaling domains. In certain embodiments, the one or more co-stimulatory signaling domains are selected from the group consisting of CD27, CD28, 4-1BB, ICOS, and OX40.

In some embodiments, the ACTR polypeptide comprises two co-stimulatory signaling domains. In some embodiments, one of the two co-stimulatory signaling domains is a CD28 co-stimulatory signaling domain and the other one is a 4-1BB co-stimulatory signaling domain, an OX40 co-stimulatory signaling domain, a CD27 co-stimulatory signaling domain, or an ICOS co-stimulatory signaling domain. In some embodiments, the other co-stimulatory signaling domain is a 4-1BB co-stimulatory signaling domain. In certain embodiments, the 4-1BB co-stimulatory signaling domain is located N-terminal to the CD28 co-stimulatory signaling domain. In certain embodiments, the 4-1BB co-stimulatory signaling domain is located C-terminal to the CD28 co-stimulatory signaling domain. In some embodiments, the other co-stimulatory signaling domain is an OX40 co-stimulatory signaling domain. In certain embodiments, the OX40 co-stimulatory signaling domain is located C-terminal to the CD28 co-stimulatory signaling domain. In certain embodiments, the OX40 co-stimulatory signaling domain is located N-terminal to the CD28 co-stimulatory signaling domain.

In some embodiments, the ACTR polypeptide contains a single (i.e., only one) co-stimulatory signaling domain. In some embodiments, the single co-stimulatory signaling domain is from CD28. In some embodiments, the transmembrane domain is a CD8 transmembrane domain. In specific embodiments, the ACTR polypeptide comprises the amino acid sequence of SEQ ID NO: 2, SEQ ID NO: 13, or SEQ ID NO: 17.

In one aspect, the present disclosure provides a nucleic acid, comprising a first nucleotide sequence encoding a first polypeptide that is any ACTR polypeptide disclosed herein, and optionally may comprise a second nucleotide sequence encoding a second polypeptide that elicits a co-stimulatory signal. In some embodiments, the second polypeptide comprises a co-stimulatory signaling domain, a co-stimulatory receptor, a binding moiety to a co-stimulatory receptor, or a ligand of a co-stimulatory receptor. In some embodiments, the second polypeptide may comprise a binding moiety (e.g., a single-chain antibody (scFv)) to 4-1BB, ICOS, OX40, CD27, or CD28. In some embodiments, the second polypeptide comprises 4-1BBL, CD80, CD86, OX40L, ICOSL, CD70, or a combination thereof. In certain embodiments, the second polypeptide may be 4-1BBL.

In some examples, the first polypeptide comprises the amino acid sequence of SEQ ID NO: 38, and/or the second polypeptide comprises the amino acid sequence of SEQ ID NO: 39. In some examples, the first polypeptide comprises the amino acid sequence of SEQ ID NO: 13, and/or the second polypeptide comprises the amino acid sequence of SEQ ID NO: 24.

In some embodiments, the nucleic acid further comprises a third nucleotide sequence located between the first nucleotide sequence and the second nucleotide sequence, wherein the third nucleotide sequence encodes a ribosomal skipping site, an internal ribosome entry site (IRES), or a second promoter. In certain embodiments, the ribosomal skipping site is a P2A peptide.

In some embodiments, the nucleic acid is in a vector. In some embodiments, the vector is an expression vector. In some embodiments, the vector is an adeno-associated virus (AAV) vector. In certain embodiments, the vector is a retroviral vector, for example, a gamma retroviral vector or a lentiviral vector.

In one aspect, the present disclosure provides a host cell comprising any nucleic acid disclosed herein. In certain embodiments, the host cell is an immune cell.

In one aspect, the present disclosure provides an immune cell (for example, a T cell or an NK cell) expressing a first polypeptide, which is any antibody-coupled T cell receptor (ACTR) disclosed herein. In some embodiments, the immune cell (for example, a T cell or an NK cell) further expresses a second polypeptide, which comprises co-stimulatory domain or a ligand of a co-stimulatory receptor. In some embodiments, the second polypeptide comprises 4-1BBL, CD80, CD86, OX40L, ICOSL, CD70, or a combination thereof. In certain embodiments, the second polypeptide comprises 4-1BBL.

In one aspect, the present disclosure provides a method for enhancing antibody-dependent cell-mediated cytotoxicity in a subject, the method comprising administering to a subject in need thereof an effective amount of any immune cell (for example, a T cell or an NK cell) or any of the vectors disclosed herein, and an effective amount of a therapeutic antibody.

In some embodiments, the therapeutic antibody is specific to TNF-alpha, HER2, CD52, CD38, BCMA, GPC3, PDGF-R-alpha, CD25, VEGF, BLyS, CD30, IL1-B, EGFR, RANK ligand, GD2, C5, CD11a, CD22, CD123, CD33, CTLA4, CEACAM5, alpha-4 integrin, CD20, CD19, IgE, RSV, VEGFR2, IL6R, IL12, IL-23, FOLR1 (folate receptor alpha), Lewis Y, PD-1, B7-H1 (PD-L1, CD274), B7-H2 (PD-DC, CD273), B7-H3, B7-H4, CD138 (Syndecan-1), integrin alpha4-beta7, or PSMA.

In some embodiments, the therapeutic antibody is selected from the group consisting of Adalimumab, Ado-Trastuzumab emtansine, Alemtuzumab, Atezolizumab, Avelumab, Basiliximab, Bevacizumab, Belimumab, Brentuximab vedotin, Canakinumab, Cetuximab, Daclizumab, Daratumumab, Denosumab, Dinutuximab, Durvalumab, Eculizumab, Efalizumab, Epratuzumab, Gemtuzumab, Golimumab, Infliximab, Ipilimumab, Labetuzumab, Natalizumab, Obinutuzumab, Ofatumumab, Olaratumab, Omalizumab, Palivizumab, Panitumumab, Pertuzumab, Ramucirumab, Rituximab, Tocilizumab, Trastuzumab, Ustekinumab, and Vedolizumab.

In some embodiments, the immune cell is an autologous T cell isolated from the subject. In other embodiments, the immune cell is an allogenic T cell. In certain embodiments, the immune cell is a T cell having the endogenous T cell receptor inhibited or eliminated.

In some embodiments, the immune cell is a T cell that is expanded and/or activated ex vivo prior to the administration.

In some embodiments, the subject is a human patient having or suspected of having cancer.

In another aspect, the present disclosure provides a method for preparing immune cells expressing an antibody-coupled T cell receptor (ACTR), the method comprising introducing any nucleic acid disclosed herein into a population of immune cells (for example, T cells or NK cells). In some embodiments, the method further comprises identifying or isolating immune cells expressing the ACTR.

In some embodiments, the nucleic acid is introduced into the immune cells (for example, T cells or NK cells) by a method selected from the group consisting of retroviral transduction, lentiviral transduction, DNA electroporation, and RNA electroporation.

In one aspect, the present disclosure provides a genetically engineered immune cell, expressing: (i) a first polypeptide which is an antibody-coupled T cell receptor (ACTR) (e.g., an ACTR comprising a CD28 cytoplasmic signaling domain); and (ii) a second polypeptide that elicits a co-stimulatory signal. In some examples, the second polypeptide may comprise a co-stimulatory receptor, a ligand thereof, or a binding moiety (e.g., a single-chain antibody) to a co-stimulatory receptor. Examples include, but are not limited to, 4-1BB, ICOS, OX40, CD27 or CD28, a ligand thereof, or a binding moiety to such a receptor.

In some embodiments, the ACTR is free of any co-stimulatory signaling domain.

In another aspect, the present disclosure provides a method of using the ACTR-expressing immune cells together with an anti-CD20 antibody for treating a solid tumor (e.g., lymphoma). In some embodiments, the method comprises: (i) administering to a subject in need thereof an effective amount of one or more lymphodepleting agents (e.g., fludarabine, cyclophosphamide, or a combination thereof); (ii) administering to the subject an anti-CD20 antibody (e.g., rituximab) after (i); and (iii) administering to the subject immune cells (e.g., T cells) expressing an antibody-coupled T cell receptor (ACTR) after (ii). The ACTR may comprise: (a) an Fc binding domain of CD16 (e.g., the CD16V isoform); (b) a co-stimulatory signaling domain of CD28, and (c), a cytoplasmic signaling domain of CD3ζ. Optionally, the ACTR may further comprise a hinge domain from CD28 and/or transmembrane domain from CD28, which is located between (a) and (b). In one example, the ACTR comprises the amino acid sequence of SEQ ID NO:9.

The subject to be treated by this method may be a human patient having a relapsed or refractory CD20+ lymphoma, for example, diffuse large B-cell lymphoma (DLBCL), mantle cell lymphoma (MCL), primary mediastinal B cell lymphoma (PMBCL), grade 3b follicular lymphoma (Gr3b-FL), and transformed histology follicular lymphoma (TH-FL). In some examples, the patient was or is under a chemotherapy for disease control. In some embodiments, the immune cells are T cells, which can be administered to the subject at a dose of 40×10⁶ cells, 80×10⁶ cells, 150×10⁶ cells, or 300×10⁶ cells. In some embodiments, the subject is administered the anti-CD20 antibody before and after step (iii).

The immune cells expressing the ACTR may be prepared by collecting immune cells from the subject and introducing a nucleic acid encoding the ACTR into the immune cells for expression of the ACTR. In some instances, the collecting step comprises leukapheresis.

Further, the present disclosure provides methods and kit for treating disease involving cells expressing a surface antigen, which also present on activated T cells. For example, provided herein is a method for inducing cytotoxicity in a subject, comprising administering to a subject in need thereof (i) an antibody specific to an antigen expressed on the surface of activated T cells (e.g., CD5, CD38, or CD7); and (ii) T cells expressing an antibody-coupled T cell receptor (ACTR). The ACTR may comprise: (a) an Fc binding domain (e.g., an extracellular ligand binding domain of an Fc receptor such as CD16); (b) a transmembrane domain (e.g., of CD28); (c) at least one co-stimulatory signaling domain (e.g., of CD28 or 4-1BB); and (d) a cytoplasmic signaling domain comprising an immunoreceptor tyrosine-based activation motif (ITAM) such as that of CD3ζ. Either (c) or (d) is located at the C-terminus of the chimeric receptor. In some examples, the ACTR may further comprise a hinge domain (e.g., that from CD28), which is located between (a) and (b). In some examples, the ACTR comprises: (a) an Fc binding domain of CD16; (b) a hinge and transmembrane domain of CD28; (c) a co-stimulatory signaling domain of CD28, and (d) a cytoplasmic signaling domain of CD3ζ. In one example, the ACTR comprises the amino acid sequence of SEQ ID NO:9. In some embodiments, the T cells expressing the ACTR are expanded in vitro.

Also provided herein is a kit comprising an antibody specific to an antigen expressed on activated T cells (e.g., CD5, CD38, or CD7), and T cells expressing an antibody-coupled T cell receptor (ACTR), for example, those described herein. In some embodiments, the T cells expressing the ACTR are expanded in vitro.

The details of one or more embodiments of the disclosure are set forth in the description below. Other features or advantages of the present disclosure will be apparent from the detailed description of several embodiments and also from the appended claims.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIG. 1 includes a series of graphs showing cytotoxicity of CD20-expressing Raji target cells incubated with T-cells expressing ACTR variants in combination with the CD20-specific antibody rituximab. A dose-dependent increase in cytotoxicity was observed with expression of nucleotides encoding ACTR variants (A) SEQ ID NO: 7, (B) SEQ ID NO: 8, (C) SEQ ID NO: 9, (D) SEQ ID NO: 13.

FIG. 2 includes a series of graphs showing cytotoxicity of HER2-expressing HCC1954 target cells incubated with T-cells expressing ACTR variants in combination with the HER2-specific antibody trastuzumab. A dose-dependent increase in cytotoxicity was observed with expression of nucleotides encoding ACTR variants (A) SEQ ID NO: 7, (B) SEQ ID NO: 8, (C) SEQ ID NO: 9, (D) SEQ ID NO: 13, and (E) SEQ ID NO: 38/SEQID NO: 39.

FIG. 3 is a set of graphs demonstrating IL-2 production by T-cells expressing ACTR variants incubated with CD20-expressing Raji target cells and the CD20-specific antibody rituximab. A dose-dependent increase in IL-2 release was observed with expression of nucleotides encoding ACTR variants (A) SEQ ID NO: 7, (B) SEQ ID NO: 8, (C) SEQ ID NO: 9, (D) SEQ ID NO: 13, and (E) SEQ ID NO: 38/SEQID NO: 39. Mock cells showed no increase in IL-2 (D, E).

FIG. 4 is a set of graphs demonstrating IL-2 production by T-cells expressing ACTR variants incubated with HER2-expressing HCC1954 target cells and the HER2-specific antibody trastuzumab. A dose-dependent increase in IL-2 release was observed with expression of nucleotides encoding ACTR variants (A) SEQ ID NO: 7, (B) SEQ ID NO: 8, (C) SEQ ID NO: 9, (D) SEQ ID NO: 13, and (E) SEQ ID NO: 38/SEQID NO: 39. Mock cells showed no increase in IL-2 (D, E).

FIG. 5 is a set of graphs demonstrating the increase in the number of CD3+ cells relative to the starting T cell count when T-cells expressing ACTR variants were incubated with the CD20-expressing Raji target cells, with or without the CD20-specific antibody rituximab, and incubated for 7 days. The increase in CD3+ cells relative to the cell count on day 0 were plotted for each condition. An antibody-dependent increase in CD3+ cells was observed with expression of nucleotides encoding ACTR variants (A) SEQ ID NO: 7, (B) SEQ ID NO: 9, (C) SEQ ID NO: 13, and (D) SEQ ID NO: 38/SEQID NO: 39.

FIG. 6 is a set of graphs demonstrating anti-tumor activity of T cells expressing ACTR variants (SEQ ID NOs: 7, 9, 13, and 38/SEQID NO: 39). Mice were inoculated with Raji tumor cells, divided into treatment groups of 5, and treated either with vehicle control (saline; open circles and solid black line), with rituximab anti-CD20 antibody alone (100 g/mouse or 5 mg/kg, IP, Day 4, 11, 18, 25; closed squares with solid gray line), ACTR T cell variant alone (1×10⁷ cells, IV, Day 5, 12; open triangles with dashed black line) or a combination of rituximab and ACTR T cell variants (closed circles with solid black line) at the same doses and days as the single agents, or T cells expressing an anti-CD19 CAR variant (1×10⁷ cells, IV, Day 5, 12; gray diamonds with dashed gray line). Mice were subsequently imaged twice weekly for tumor bioluminescence using an IVIS Spectrum. Tumor burden, expressed as photons/sec, is plotted over time. ** p<0.01, **** p<0.0001 (compared to rituximab control, 2-way ANOVA with Sidac's multiple comparison test) FIG. 7 is a graph demonstrating proliferation of CD3-positive T cells after repeated stimulation with rituximab-opsonized target cells every 3-4 days with T cells expressing nucleic acids encoding ACTR variants SEQ ID NO: 7, SEQ ID NO: 8, SEQ ID NO: 9, SEQ ID NO: 13, SEQ ID NO: 38/SEQID NO: 39, and CD19 CAR.

FIG. 8 is a set of graphs demonstrating cytotoxicity against rituximab opsonized Raji target cells after each restimulation round with T cells expressing nucleic acids encoding ACTR variants SEQ ID NO: 7, SEQ ID NO: 8, SEQ ID NO: 9, SEQ ID NO: 13, and SEQ ID NO: 38/SEQID NO: 39, and CD19 CAR. The fold change in Raji target cell number relative to the number of target cells at the previous time point are plotted as a function of time. The data is plotted as a line graph (A) and a bar graph (B). Values below 1 indicate control of target cell growth and cytotoxicity.

FIG. 9 is a graph demonstrating ACTR variant SEQ ID NO: 9 activity in a repeated simulation “stress test” where ACTR variant expressing T cells were challenged with fresh CD20+ Ramos tumor cells and rituximab every 3-4 days. The total T cell and target cell counts are plotted as a function of time.

FIG. 10 is a graph demonstrating the T cell activity (IL-2 release, proliferation) of ACTR variant SEQ ID NO: 9 expressing T cells in combination with tumor targeting antibodies across a plurality of cells lines and indications.

FIG. 11 is a set of graphs demonstrating the results of binding assays using rituximab, CD20+ tumor cells, and ACTR variant SEQ ID NO: 9. FIG. 11, panel A demonstrates binding of rituximab to CD20+ lymphoma tumor cells Raji, Daudi, and RL and little to no binding to the CD20-negative cell line K562 as determined by flow cytometry. FIG. 11, panel B, demonstrates binding of rituximab to T cells expressing ACTR variant SEQ ID NO: 9 as determined by flow cytometry.

FIG. 12 is a graphic depicting a hypothetical model for ACTR expressing T cell activation. Similar to low-affinity natural T cell receptors and Fc receptors, ACTR T cells are activated via structural avidity when ACTR engages multiple rituximab molecules bound to the surface of tumor cells.

FIG. 13 is a set of graphs demonstrating (A, B) the results of cytotoxicity assays using CD20+ tumor cells in the presence of increasing cell doses of either Mock or ACTR variant SEQ ID NO: 9 expressing T cells and 1 μg/mL rituximab; and (C) the results of cytotoxicity using CD20+ tumor cells in the presence of ACTR expressing T cells and increasing concentrations of rituximab at a 2:1 E:T (effector to target cell) ratio.

FIG. 14 is a set of graphs demonstrating (A, B) results of cytokine release assays for T cells expressing ACTR variant SEQ ID NO: 9 in the presence of CD20+ tumor cells and increasing concentrations of rituximab at a 2:1 E:T (effector to target cell) ratio; and (C) results of proliferation assays of ACTR variant SEQ ID NO: 9 expressing T cells in the presence of CD20+ tumor cells and increasing concentrations of rituximab at a 1:1 E:T (effector to target cell) ratio.

FIG. 15 is a set of graphs demonstrating results of (A) cytotoxicity, (B) IL-2 production, and (C) T cell proliferation assays of ACTR variant SEQ ID NO: 9 expressing T cells in the presence of CD20+ Ramos or CD20-negative tumor cells and either rituximab or trastuzumab (anti-HER2). Antibodies were used at 4 μg/mL final concentration. These graphs demonstrate that robust ACTR T cell activity is only observed in the presence of antibody (rituximab) and a target expressing a cognate antigen (CD20-expressing Ramos cells).

FIG. 16 is a graph demonstrating the results of cytokine production assays for mock T cells and ACTR variant SEQ ID NO: 9 expressing T cells in the presence of Raji cells, 1 g/mL rituximab, and increasing concentrations (0 mg/mL, 0.4 mg/mL, 1.2 mg/mL) of human IgG (up to 3600 fold rituximab).

FIG. 17 is a set of graphs demonstrating (A) flow cytometry quantification of IL-6R on normal immune cells and the multiple myeloma cell line NCI-H929; and (B) the results of binding assays of tocilizumab (anti-IL-6R antibody) to T cells expressing ACTR variant SEQ ID NO: 9 or to mock T cells.

FIG. 18 is a set of graphs demonstrating the results of (A) cytotoxicity and (B) cytokine release assays in the absence of T cells or in the presence of ACTR variant SEQ ID NO: 9 expressing T cells or mock T cells with IL-6R+NCI-H929 cells and increasing concentrations of tocilizumab (0-25 μg/mL). ACTR variant SEQ ID NO: 9 expressing T cells in the presence of NCI-H929 cells and an anti-CD38 positive control antibody (1.5 μg/mL) are shown under the same conditions as a positive control.

FIG. 19 is a graph demonstrating percentage survival of mice with an aggressive Raji xenograft using ACTR variant SEQ ID NO: 9 expressing T cells in combination with different doses of rituximab, T cells expressing anti-CD19-CAR, rituximab alone, ACTR variant SEQ ID NO: 9 expressing T cells alone, or a control treatment. Rituximab was dosed on day 4 after tumor inoculation and weekly thereafter for a total of 4 doses. A 1×10⁷ dose of T cells (ACTR or CAR) were administered on day 5 post-tumor inoculation.

FIG. 20 is a graph demonstrating percentage survival of mice with an aggressive Raji xenograft using one or two doses of ACTR variant SEQ ID NO: 9 expressing T cells in combination with rituximab, rituximab alone, ACTR variant SEQ ID NO: 9 expressing T cells alone, or a control treatment. Rituximab (100 μg) was dosed on day 4 after tumor inoculation and weekly thereafter for a total of 4 doses. One or two doses of 1×10⁷ ACTR variant SEQ ID NO: 9 expressing T cells were given on day 5 or day 5 and day 12, one day following rituximab administration.

FIG. 21 is a set of graphs demonstrating the results of experiments using T cells from three unique Non-Hodgkin Lymphoma (NHL) donors (NHL1, NHL2, and NHL3) expressing ACTR variant SEQ ID NO: 9. FIG. 21, panel A demonstrates expression level of ACTR variant SEQ ID NO: 9 in T cells generated from PBMCs of three unique Non-Hodgkin Lymphoma (NHL) donors. Cytokine release by ACTR variant SEQ ID NO: 9-expressing T cells in the presence of CD20+ tumor cells and increasing concentrations of rituximab at a 1:1 E:T is shown in FIG. 21, panel B. ACTR variant SEQ ID NO: 9-expressing T cell proliferation in the presence of CD20+ tumor cells and increasing concentrations of rituximab at a 1:1 E:T ratio is shown in FIG. 21, panel C.

FIG. 22 is a graphic depicting an exemplary treatment schedule for patients having relapsed or refractory CD20+ B cell lymphoma with ACTR expressing T cells in combination with rituximab as an exemplary therapeutic antibody.

FIG. 23 is a graph depicting HER2 protein expression on HER2-amplified cell lines (OE19, N87, and SKBR3) and non-HER2-amplified cell lines (MCF7 and KATOIII). The HER2 expression level was determined by flow cytometry after staining with an anti-human HER2 antibody. Mean fluorescence intensity (MFI) of stained cells is plotted for each cell line.

FIG. 24 is a set of graphs demonstrating the results of assays for (A) cytotoxicity, (B) cytokine production, and (C) T cell proliferation for ACTR variant SEQ ID NO: 9 expressing T cells in the presence of HER2-amplified and non-HER2 amplified cell lines and trastuzumab (anti-HER2). Antibodies were used at 5 μg/mL final concentration.

FIG. 25 is a set of graphs demonstrating the results of assays for (A) cytotoxicity, (B) cytokine production, and (C) T cell proliferation of trastuzumab-based HER2-targeting CAR-T cells in the presence of HER2-amplified and non-HER2 amplified cell lines.

FIG. 26 is a set of graphs demonstrating the results of assays for (A) proliferation of ACTR variant SEQ ID NO: 9 expressing T cells in the presence of trastuzumab and HER2-amplified and non-HER2 amplified cell lines; and (B) proliferation of HER2-targeting CAR-T cells in the presence of HER2-amplified and non-HER2 amplified cell lines. Total T cell input on Day 0 was 100,000 cells. T cells were quantified on Day 6 by flow cytometry after staining for CD3 and total T cells are plotted as a function of antibody concentration for each target cell line for experiments with ACTR (A) or as a function of target cell for experiments with CAR T cells (B).

FIG. 27 is a graphic depicting an in vivo dosing regimen for ACTR variant SEQ ID NO: 9 expressing T cells in combination with trastuzumab in female NOD.Cg-Prkdc^{scid} IL-2rg^{tm1WJl}/SzJ mice with subcutaneous N87 gastric xenografted tumors of approximately 80 mm³ starting volume. Trastuzumab (100 μg/mouse, IP) was dosed once weekly for four weeks starting 7 days after tumor inoculation. ACTR variant SEQ ID NO: 9 expressing T cells or trastuzumab-based HER2-targeting CAR-T cells (1.5×10⁷ total T cells) were dosed once weekly for 2 weeks starting on Day 8 after tumor cell inoculation. Control mice were administered vehicle alone on the same schedule.

FIG. 28 is a graph depicting the results of the experiment shown in FIG. 27. Mean tumor volume is plotted as a function of time for treatment groups: vehicle, trastuzumab alone, ACTR variant SEQ ID NO: 9 expressing T cells alone, ACTR variant SEQ ID NO: 9 expressing T cells with trastuzumab, and anti-HER2 CAR T cells.

FIG. 29 is a graphic depicting HER2 expression on HER2-amplified tumor line (N87) cells; non-HER-amplified tumor line (MCF7) cells; HER-2 negative cell line (Daudi) cells; and various normal cell lines (mammary epithelium, pulmonary artery smooth muscle, cardiac myocytes, bronchial epithelium, or renal epithelium). HER2 levels were measured by flow cytometry after staining with an anti-human HER2 antibody. Mean fluorescence intensity (MFI) of stained cells is represented.

FIG. 30 is a set of graphs depicting the results of a cytotoxicity assay using (A) ACTR variant SEQ ID NO: 9 expressing T cells in combination with trastuzumab (5 μg/mL); or (B) trastuzumab-based HER2-targeting CAR-T cells. Target cells are N87 (HER2 amplified), MCF7 (HER2 low), Daudi (HER2 negative), and normal cell lines (mammary epithelium, pulmonary artery smooth muscle, cardiac myocytes, bronchial epithelium, or renal epithelium).

FIG. 31 is a set of graphs depicting cytokine release profiles (IL-2) of ACTR variant SEQ ID NO: 9 expressing T cells in the presence of trastuzumab (A) and anti-HER2 CAR T cells (B) and normal primary cells (mammary epithelium, pulmonary artery smooth muscle, cardiac myocytes, bronchial epithelium, or renal epithelium). HER2-amplified tumor line (N87) cells; non-HER-amplified tumor line (MCF7) cells; and HER-2 negative cell line (Daudi) cells are shown as controls.

FIG. 32 is a set of graphs depicting cytokine release profiles (IFN-γ) of ACTR variant SEQ ID NO: 9 expressing T cells in the presence of trastuzumab (A) and anti-HER2 CAR T cells (B) and normal primary cells (mammary epithelium, pulmonary artery smooth muscle, cardiac myocytes, bronchial epithelium, or renal epithelium). HER2-amplified tumor line (N87) cells; non-HER-amplified tumor line (MCF7) cells; and HER-2 negative cell line (Daudi) cells are shown as controls.

FIG. 33 is a series of graphs depicting CD38 expression on the surface of (A) multiple myeloma and lymphoma cell lines (with U266B1 as a CD38-negative control cell line); (B) plasma cells from a multiple myeloma patient in comparison with RPMI-8226, KMS-20, and NCI-H929 multiple myeloma cell lines; (C) PBMC subsets from two healthy donors; and (D) red blood cells from five healthy donors as determined by flow cytometry. Mean fluorescence intensity is plotted for each cell type evaluated.

FIG. 34 is a set of graphs depicting CD38 expression on the surface of activated ACTR variant SEQ ID NO: 9 expressing T cells or Daudi cells as measured by flow cytometry. Results are shown for ACTR variant SEQ ID NO: 9 expressing T cells stimulated with Daudi target cells or with rituximab (1 μg/mL). The mean fluorescence intensity is plotted as a function of time for total T cells (A) and ACTR-positive T cells (B) and compared to the mean fluorescence intensity of Daudi target cells.

FIG. 35 is a set of graphs depicting fold expansion (A), viability (B), cell size (C), and CD38 expression (D) of T cells activated with anti-human CD3 and anti-human CD28 activating antibodies and expanded for ten days in the presence of 100 U/mL IL-2. ACTR variant SEQ ID NO: 9 expressing T cells, and CD38-targeting THB7 CAR T cells were transduced with virus encoding ACTR or CAR, as appropriate, on day 3; mock T cells were not transduced.

FIG. 36 is a set of graphs depicting cytotoxicity observed for mock or ACTR variant SEQ ID NO: 9 expressing T cells cultured with (A) NCI-H929, (B) MM.1S, (C) RPMI-8226, or (D) Daudi target cells in the presence of daratumumab. Percent cytotoxicity is plotted as a function of antibody concentration.

FIG. 37 is a set of graphs depicting IL-2 (A) and IFN-γ (B) production from ACTR variant SEQ ID NO: 9 expressing T cell incubated in the presence of daratumumab-opsonized NCI-H929, MM.1S, RPMI-8226, and Daudi target cells. Cytokine production with mock T cells (not plotted) was below the linear range of the standard curve.

FIG. 38 is a set of graphs depicting the results of proliferation assays for mock or ACTR variant SEQ ID NO: 9 expressing T cells in the presence of daratumumab-opsonized NCI-H929, MM. 1S, RPMI-8226, and Daudi target cells. Total T cell count is plotted as a function of daratumumab antibody concentration in panels A and B. In FIG. 38, panel C, the frequency (%) of CD16+ cells was calculated within the total CD3+ T cell gate, and plotted as a function of daratumumab antibody concentration.

FIG. 39 is a set of graphs depicting the results of antibody-specific cytotoxicity assays for mock (A) or ACTR variant SEQ ID NO: 9 expressing (B) T cells incubated with donor-matched PBMCs and RPMI-8226 multiple myeloma cells (MM cells) and 1 μg/mL or 10 μg/mL daratumumab. Reactions were analyzed by flow cytometry to isolate the cytotoxic effect on different PBMC subtypes. The percent cytotoxicity is plotted as a function of cell type.

FIG. 40 is a set of graphs depicting the results of IFN-γ (A) and IL-2 (B) release assays for ACTR variant SEQ ID NO: 9 expressing T cells in the presence of autologous PBMCs or in the presence of autologous PBMCs and RPMI-8226 target cells and increasing concentrations of daratumumab.

FIG. 41 is a set of graphs depicting the evaluation of CD38 expression on red blood cells by flow cytometry (A); the evaluation of daratumumab binding to red blood cells by flow cytometry (B); and the evaluation of ACTR variant SEQ ID NO: 9 expressing T cell mediated hemolysis in the presence of varying concentrations of daratumumab using an ACTR and red blood cell co-culture assay.

FIG. 42 is a graph depicting average IL-2 production relative to that with ACTR variant SEQ ID NO: 2 from T cells from two different donors expressing ACTR variants SEQ ID NO: 2, 9, 13, 19, 20, 21, 22, and 27 in the presence of trastuzumab and Her2-expressing HCC1954 or SKBR3 target cells.

FIG. 43 is a set of graphs depicting IL-2 (A) and IFN-γ (B) release with mock T cells and T cells expressing ACTR variants SEQ ID NO: 9 and SEQ ID NO: 26 in the presence of increasing concentrations of the HER2-targeting antibody trastuzumab and HER2-expressing targets BT20 and SKBR3.

FIG. 44 is a set of graphs depicting fold expansion (A), cell size (B), and viability (C) as a function of time for T cells activated with anti-human CD3 and anti-human CD28 activating antibodies and expanded for ten days in the presence of 100 U/mL IL-2. ACTR variant SEQ ID NO: 9 expressing T cells, and CD38-targeting THB7 CAR and 056 CAR T cells were transduced with virus encoding ACTR or CAR, as appropriate, on day 3; mock T cells were not transduced.

FIG. 45 is a set of graphs depicting CD38 expression at day 6, 8, and 10 on T cells activated with anti-human CD3 and anti-human CD28 activating antibodies and expanded for ten days in the presence of 100 U/mL IL-2, as measured by flow cytometry. ACTR variant SEQ ID NO: 9 expressing T cells, and CD38-targeting THB7 CAR and 056 CAR T cells were transduced with virus encoding ACTR or CAR, as appropriate, on day 3; mock T cells were not transduced.

FIG. 46 is a set of graphs depicting cytotoxicity observed for mock T cells, ACTR variant SEQ ID NO: 9 expressing T cells, THB7 CAR T cells, and 056 CAR T cells cultured with (A) Daudi or (B) NCI-H929 target cells in the absence or presence of daratumumab. Percent cytotoxicity is plotted as a function of effector:target (E:T) ratio.

FIG. 47 is a set of graphs depicting IFN-γ (A) and IL-2 (B) production for mock T cells in the presence of daratumumab, ACTR variant SEQ ID NO: 9 expressing T cells in the presence of daratumumab, THB7 CAR T cells, and 056 CAR T cells cultured with NCI-H929, RPMI-8226, or Daudi target cells.

### DETAILED DESCRIPTION OF DISCLOSURE

Antibody-based immunotherapies are used to treat a wide variety of diseases, including many types of cancer. Such a therapy often depends on recognition of cell surface molecules that are differentially expressed on cells for which elimination is desired (e.g., target cells such as cancer cells) relative to normal cells (e.g., non-cancer cells) (Weiner et al. Cell (2012) 148(6): 1081-1084). Several antibody-based immunotherapies have been shown in vitro to facilitate antibody-dependent cell-mediated cytotoxicity of target cells (e.g. cancer cells), and for some it is generally considered that this is the mechanism of action in vivo, as well. ADCC is a cell-mediated innate immune mechanism whereby an effector cell of the immune system, such as natural killer (NK) cells, T cells, monocyte cells, macrophages, or eosinophils, actively lyses target cells (e.g., cancer cells) recognized by specific antibodies.

The present disclosure is also based, at least in part, on the unexpected findings that ACTR polypeptides comprising a CD16A extracellular domain and a CD28 co-stimulatory domain or ACTR polypeptides comprising a CD16A extracellular domain and a shortened hinge domain or no hinge domain exhibited superior bioactivity. Further, the present disclosure is also based on the findings that the ACTR technology as described herein overcame the in vitro expansion/manufacturing problems associated with conventional CAR-T cells that target antigens presenting on activated T cells due to fratricide effects. In combination with antibodies specific to such antigens, ACTR-T cells can be used to treat diseases associated with cells expressing surface antigens, which also present on activated T cells, such as CD5, CD38, or CD7.

Accordingly, the present disclosure provides improved ACTR polypeptides, a genetically engineered immune cell expressing such, and a method of enhancing antibody-dependent cell cytotoxicity (ADCC) in a subject using a combination therapy comprising a therapeutically effective amount of a therapeutic antibody and a therapeutically effective amount of immune cells (e.g., T lymphocytes or NK lymphocytes) that express an ACTR polypeptide as described herein. The present disclosure also provides immune cells (e.g., T lymphocytes and/or NK cells) that express an ACTR polypeptide and another exogenous polypeptide capable of eliciting a co-stimulatory signal.

As used herein, an ACTR polypeptide or construct refers to a non-naturally occurring molecule that can be expressed on the surface of a host cell and comprises an extracellular domain (e.g., a CD16A extracellular domain) capable of binding to a target molecule containing an Fc portion and one or more cytoplasmic signaling domains for triggering effector functions of the immune cell expressing the ACTR polypeptide, wherein at least two domains of the ACTR polypeptide may be derived from different molecules. The ACTR polypeptide may comprise a CD16A extracellular domain capable of binding to a target molecule containing an Fc portion, a transmembrane domain, one or more co-stimulatory signaling domains, and a CD3ζ cytoplasmic signaling domain. At least one of the co-stimulatory signaling domains may be a CD28 co-stimulatory domain. The ACTR polypeptide can either be free of a hinge domain from any non-CD16A receptor or comprise more than one co-stimulatory signaling domain if the transmembrane domain is a CD8 transmembrane domain.

Antibodies for use with the described methods can bind to a protein on the surface of a target cell (e.g., a cancer cell). Immune cells that express receptors capable of binding such Fc-containing molecules, for example the ACTR polypeptide molecules described herein, recognize the target cell-bound antibodies and this receptor/antibody engagement stimulates the immune cell to perform effector functions such as release of cytotoxic granules or expression of cell-death-inducing molecules, leading to enhanced cell toxicity of the target cells.

The ACTR polypeptides, cells, and methods described herein would confer a number of advantages. For example, via the CD16A extracellular domain that binds Fc, the ACTR polypeptides described herein can bind to the Fc portion of the antibodies bound to target cells rather than directly binding a specific target antigen (e.g., a cancer antigen). Thus, immune cells expressing the ACTR polypeptides described herein would be able to induce/enhance cell death of any type of cells that are bound by the therapeutic antibody. Further, the improved ACTR constructs were shown to exhibit superior bioactivities as described herein. Thus, combined therapies involving immune cells expressing such improved ACTR constructs and therapeutic antibodies would be expected to exert superior therapeutic effects on target disease cells, such as cancer cells.

I. ACTR Constructs

In some embodiments, the ACTR constructs (also called ACTR polypeptides) described herein comprise an extracellular domain with binding affinity and specificity for the Fc portion of an immunoglobulin (“Fc binder” or “Fc binding domain”), a transmembrane domain, and a cytoplasmic signaling domain comprising an immunoreceptor tyrosine-based activation motif (ITAM). In some embodiments, the ACTR polypeptides described herein may further include at least one co-stimulatory signaling domain. The ACTR polypeptides are configured such that, when expressed on a host cell, the extracellular ligand-binding domain is located extracellularly for binding to a target molecule and the ITAM-containing cytoplasmic signaling domain. The optional co-stimulatory signaling domain may be located in the cytoplasm for triggering activation and/or effector signaling. In some embodiments, an ACTR polypeptide as described herein may comprise, from N-terminus to C-terminus, the Fc binding domain, the transmembrane domain, and the ITAM-containing cytoplasmic signaling domain. In some embodiments, an ACTR polypeptide as described herein comprises, from N-terminus to C-terminus, the Fc binding domain, the transmembrane domain, at least one co-stimulatory signaling domain, and the ITAM-containing cytoplasmic signaling domain. In other embodiments, an ACTR polypeptide as described herein comprises, from N-terminus to C-terminus, the Fc binding domain, the transmembrane domain, the ITAM-containing cytoplasmic signaling domains, and at least one co-stimulatory signaling domain.

Exemplary ACTR constructs for use with the methods and compositions described herein may be found, for example, in the instant description and figures or may be found in PCT Patent Publication No.: WO2016040441A1, which is incorporated by reference herein for this purpose.

The improved ACTR polypeptides described herein may comprise a CD16A extracellular domain with binding affinity and specificity for the Fc portion of an immunoglobulin (“Fc binder” or “Fc binding domain”), a transmembrane domain, and a CD3ζ cytoplasmic signaling domain. In some embodiments, the ACTR polypeptides may further include one or more co-stimulatory signaling domains, at least one of which is a CD28 co-stimulatory signaling domain. The ACTR polypeptides are configured such that, when expressed on a host cell, the extracellular ligand-binding domain is located extracellularly for binding to a target molecule and the CD3ζ cytoplasmic signaling domain. The co-stimulatory signaling domain may be located in the cytoplasm for triggering activation and/or effector signaling. In some embodiments, an ACTR polypeptide as described herein may comprise, from N-terminus to C-terminus, the Fc binding domain such as a CD16A extracellular domain, the transmembrane domain, the optional one or more co-stimulatory domains (e.g., a CD28 co-stimulatory domain, a 4-1BB co-stimulatory signaling domain, an OX40 co-stimulatory signaling domain, a CD27 co-stimulatory signaling domain, or an ICOS co-stimulatory signaling domain), and the CD3ζ cytoplasmic signaling domain.

As used in this specification, the phrase “a protein X transmembrane domain” (e.g., a CD8 transmembrane domain) refers to any portion of a given protein, i.e., transmembrane-spanning protein X, that is thermodynamically stable in a membrane.

As used in this specification, the phrase “a protein X cytoplasmic signaling domain,” for example, a CD3 cytoplasmic signaling domain, refers to any portion of a protein (protein X) that interacts with the interior of a cell or organelle and is capable of relaying a signal.

As used in this specification, the phrase “a protein X co-stimulatory signaling domain,” e.g., a CD28 co-stimulatory signaling domain, refers to the portion of a given co-stimulatory protein (protein X, such as CD28, 4-1BB, OX40, CD27, or ICOS) that can transduce co-stimulatory signals into immune cells (such as T cells).

In some embodiments, if the transmembrane domain of the ACTR polypeptide is a CD8 transmembrane domain, the ACTR polypeptide may be free of a hinge domain from any non-CD16A receptor or contain a shortened hinge domain. Alternatively or in addition, the ACTR polypeptide may comprise more than one co-stimulatory signaling domain.

In some embodiments, ACTR polypeptides described herein may further comprise a hinge domain, which may be located at the C-terminus of the Fc binding domain and the N-terminus of the transmembrane domain. In other embodiments, the ACTR polypeptide described herein may have no non-CD16A hinge domain, or contain no hinge domain at all. In yet other embodiments, the ACTR polypeptide described herein may have a shortened hinge domain (e.g., including up to 25 amino acid residues).

Alternatively or in addition, the ACTR polypeptides described herein may contain two or more co-stimulatory signaling domains, which may link to each other or be separated by the ITAM-containing cytoplasmic signaling domain. The extracellular Fc binder, transmembrane domain, optional co-stimulatory signaling domain(s), and ITAM-containing cytoplasmic signaling domain in an ACTR polypeptide may be linked to each other directly, or via a peptide linker. In some embodiments, any of the ACTR polypeptides described herein may comprise a signal sequence at the N-terminus.

As used in this specification and the appended claims, the singular forms “a,” “an,” and “the” include plural references unless the context clearly dictates otherwise.

### A. Fc Binding Domains

The ACTR polypeptides described herein comprise an extracellular domain that is an Fc binding domain, i.e., capable of binding to the Fc portion of an immunoglobulin (e.g., IgG, IgA, IgM, or IgE) of a suitable mammal (e.g., human, mouse, rat, goat, sheep, or monkey). Suitable Fc binding domains may be derived from naturally occurring proteins such as mammalian Fc receptors or certain bacterial proteins (e.g., protein A, protein G). Additionally, Fc binding domains may be synthetic polypeptides engineered specifically to bind the Fc portion of any of the antibodies described herein with high affinity and specificity. For example, such an Fc binding domain can be an antibody or an antigen-binding fragment thereof that specifically binds the Fc portion of an immunoglobulin. Examples include, but are not limited to, a single-chain variable fragment (scFv), a domain antibody, or a nanobody. Alternatively, an Fc binding domain can be a synthetic peptide that specifically binds the Fc portion, such as a Kunitz domain, a small modular immunopharmaceutical (SMIP), an adnectin, an avimer, an affibody, a DARPin, or an anticalin, which may be identified by screening a peptide combinatory library for binding activities to Fc.

In some embodiments, the Fc binding domain is an extracellular ligand-binding domain of a mammalian Fc receptor. As used herein, an “Fc receptor” is a cell surface bound receptor that is expressed on the surface of many immune cells (including B cells, dendritic cells, natural killer (NK) cells, macrophage, neutrophils, mast cells, and eosinophils) and exhibits binding specificity to the Fc domain of an antibody. Fc receptors are typically comprised of at least two immunoglobulin (Ig)-like domains with binding specificity to an Fc (fragment crystallizable) portion of an antibody. In some instances, binding of an Fc receptor to an Fc portion of the antibody may trigger antibody dependent cell-mediated cytotoxicity (ADCC) effects. The Fc receptor used for constructing an ACTR polypeptide as described herein may be a naturally-occurring polymorphism variant (e.g., the CD16 V158 variant), which may have increased or decreased affinity to Fc as compared to a wild-type counterpart. Alternatively, the Fc receptor may be a functional variant of a wild-type counterpart, which carry one or more mutations (e.g., up to 10 amino acid residue substitutions including 1, 2, 3, 4, 5, 6, 7, 8, 9, or 10 mutations) that alter the binding affinity to the Fc portion of an Ig molecule. In some instances, the mutation may alter the glycosylation pattern of the Fc receptor and thus the binding affinity to Fc.

The table below lists a number of exemplary polymorphisms in Fc receptor extracellular domains (see, e.g., Kim et al., J. Mol. Evol. 53:1-9, 2001) which may be used in any of the methods or constructs described herein:

TABLE 1
Exemplary Polymorphisms in Fc Receptors

| Amino Acid | | | | | | | | | | |
|---|---|---|---|---|---|---|---|---|---|---|
| Number | 19 | 48 | 65 | 89 | 105 | 130 | 134 | 141 | 142 | 158 |
| FCR10 | R | S | D | I | D | G | F | Y | T | V |
| P08637 | R | S | D | I | D | G | F | Y | I | F |
| S76824 | R | S | D | I | D | G | F | Y | I | V |
| J04162 | R | N | D | V | D | D | F | H | I | V |
| M31936 | S | S | N | I | D | D | F | H | I | V |
| M24854 | S | S | N | I | E | D | S | H | I | V |
| X07934 | R | S | N | I | D | D | F | H | I | V |
| X14356 (FcγRII) | N | N | N | S | E | S | S | S | I | I |
| M31932 (FcγRI) | S | T | N | R | E | A | F | T | I | G |
| X06948 (FcαϵI) | R | S | E | S | Q | S | E | S | I | V |

Fc receptors are classified based on the isotype of the antibody to which it is able to bind. For example, Fc-gamma receptors (FcγR) generally bind to IgG antibodies, such as one or more subtype thereof (i.e., IgG1, IgG2, IgG3, IgG4); Fc-alpha receptors (FcαR) generally bind to IgA antibodies; and Fc-epsilon receptors (FcεR) generally bind to IgE antibodies. In some embodiments, the Fc receptor is an Fc-gamma receptor, an Fc-alpha receptor, or an Fc-epsilon receptor. Examples of Fc-gamma receptors include, without limitation, CD64A, CD64B, CD64C, CD32A, CD32B, CD16A, and CD16B. An example of an Fc-alpha receptor is FcαR1/CD89. Examples of Fc-epsilon receptors include, without limitation, FcεRI and FcεRII/CD23. The table below lists exemplary Fc receptors for use in constructing the ACTR polypeptides described herein and their binding activity to corresponding Fc domains:

TABLE 2
Exemplary Fc Receptors

| | Principal | |
|---|---|---|
| Receptor name | antibody ligand | Affinity for ligand |
| FcγRI (CD64) | IgG1 and IgG3 | High (Kd~10⁻⁹M) |
| FcγRIIA (CD32) | IgG | Low (Kd > 10⁻⁷M) |
| FcγRIIB1 (CD32) | IgG | Low (Kd > 10⁻⁷M) |
| FcγRIIB2 (CD32) | IgG | Low (Kd > 10⁻⁷M) |
| FcγRIIIA (CD16a) | IgG | Low (Kd > 10⁻⁶M) |
| FcγRIIIB (CD16b) | IgG | Low (Kd > 10⁻⁶M) |
| FcεRI | IgE | High (Kd~10⁻¹⁰M) |
| FcεRII (CD23) | IgE | Low (Kd > 10⁻⁷M) |
| FcαRI (CD89) | IgA | Low (Kd > 10⁻⁶M) |
| Fcα/μR | IgA and IgM | High for IgM, Mid for IgA |
| FcRn | IgG | |

Selection of the ligand binding domain of an Fc receptor for use in the ACTR polypeptides described herein will be apparent to one of skill in the art. For example, it may depend on factors such as the isotype of the antibody to which binding of the Fc receptor is desired and the desired affinity of the binding interaction.

In some examples, the Fc binding domain is the extracellular ligand-binding domain of CD16, which may incorporate a naturally occurring polymorphism that may modulate affinity for Fc. In some examples, the Fc binding domain is the extracellular ligand-binding domain of CD16 incorporating a polymorphism at position 158 (e.g., valine or phenylalanine). In some embodiments, the Fc binding domain is produced under conditions that alter its glycosylation state and its affinity for Fc.

The amino acid sequences of human CD16A F158 and CD16A V158 variants are provided below with the F158 and V158 residue highlighted in bold/face and underlined (signal peptide italicized):

CD16A F158 (SEQ ID NO: 36):
MWQLLLPTALLLLVSAGMRTEDLPKAVVFLEPQWYRVLEKDSVTLKCQ
GAYSPEDNSTQWFHNESLISSQASSYFIDAATVDDSGEYRCQTNLSTL
SDPVQLEVHIGWLLLQAPRWVFKEEDPIHLRCHSWKNTALHKVTYLQN
GKGRKYFHHNSDFYIPKATLKDSGSYFCRGLFGSKNVSSETVNITITQ
GLAVSTISSFFPPGYQ
CD16A V158 (SEQ ID NO: 37):
MWQLLLPTALLLLVSAGMRTEDLPKAVVFLEPQWYRVLEKDSVTLKCQ
GAYSPEDNSTQWFHNESLISSQASSYFIDAATVDDSGEYRCQTNLSTL
SDPVQLEVHIGWLLLQAPRWVFKEEDPIHLRCHSWKNTALHKVTYLQN
GKGRKYFHHNSDFYIPKATLKDSGSYFCRGLVGSKNVSSETVNITITQ
GLAVSTISSFFPPGYQVSFCLVMVLLFAVDTGLYFSVKTNIRSSTRDW
KDHKFKWRKDPQDK

In some embodiments, the Fc binding domain is the extracellular ligand-binding domain of CD16 incorporating modifications that render the ACTR polypeptide specific for a subset of IgG antibodies. For example, mutations that increase or decrease the affinity for an IgG subtype (e.g., IgG1) may be incorporated.

Any of the Fc binding domains described herein may have a suitable binding affinity for the Fc portion of a therapeutic antibody. As used herein, “binding affinity” refers to the apparent association constant or K_{A}. The K_{A} is the reciprocal of the dissociation constant, K_{D}. The extracellular ligand-binding domain of an Fc receptor domain of the ACTR polypeptides described herein may have a binding affinity K_{d} of at least 10⁻⁵, 10⁻⁶, 10⁻⁷, 10⁻⁸, 10⁻⁹, 10⁻¹⁰ M or lower for the Fc portion of antibody. In some embodiments, the Fc binding domain has a high binding affinity for an antibody, isotype(s) of antibodies, or subtype(s) thereof, as compared to the binding affinity of the Fc binding domain to another antibody, isotype(s) of antibodies, or subtypes(s) thereof. In some embodiments, the extracellular ligand-binding domain of an Fc receptor has specificity for an antibody, isotype(s) of antibodies, or subtype(s) thereof, as compared to binding of the extracellular ligand-binding domain of an Fc receptor to another antibody, isotype(s) of antibodies, or subtypes(s) thereof.

Other Fc binding domains as known in the art may also be used in the ACTR constructs described herein including, for example, those described in WO2015058018A1, the relevant disclosures of which are incorporated by reference for the purpose and subject matter referenced herein.

### B. Transmembrane Domain

The transmembrane domain of the ACTR polypeptides described herein can be in any form known in the art. As used herein, a “transmembrane domain” refers to any protein structure that is thermodynamically stable in a cell membrane, preferably a eukaryotic cell membrane. A transmembrane domain compatible for use in the ACTR polypeptides used herein may be obtained from a naturally occurring protein. Alternatively, it can be a synthetic, non-naturally occurring protein segment, e.g., a hydrophobic protein segment that is thermodynamically stable in a cell membrane.

Transmembrane domains are classified based on the three dimensional structure of the transmembrane domain. For example, transmembrane domains may form an alpha helix, a complex of more than one alpha helix, a beta-barrel, or any other stable structure capable of spanning the phospholipid bilayer of a cell. Furthermore, transmembrane domains may also or alternatively be classified based on the transmembrane domain topology, including the number of passes that the transmembrane domain makes across the membrane and the orientation of the protein. For example, single-pass membrane proteins cross the cell membrane once, and multi-pass membrane proteins cross the cell membrane at least twice (e.g., 2, 3, 4, 5, 6, 7 or more times).

Membrane proteins may be defined as Type I, Type II or Type III depending upon the topology of their termini and membrane-passing segment(s) relative to the inside and outside of the cell. Type I membrane proteins have a single membrane-spanning region and are oriented such that the N-terminus of the protein is present on the extracellular side of the lipid bilayer of the cell and the C-terminus of the protein is present on the cytoplasmic side. Type II membrane proteins also have a single membrane-spanning region but are oriented such that the C-terminus of the protein is present on the extracellular side of the lipid bilayer of the cell and the N-terminus of the protein is present on the cytoplasmic side. Type III membrane proteins have multiple membrane-spanning segments and may be further sub-classified based on the number of transmembrane segments and the location of N- and C-termini.

In some embodiments, the transmembrane domain of the ACTR polypeptide described herein is derived from a Type I single-pass membrane protein. Single-pass membrane proteins include, but are not limited to, CD8α, CD8β, 4-1BB/CD137, CD27, CD28, CD34, CD4, FcεRIγ, CD16, OX40/CD134, CD3ζ, CD3ε, CD3γ, CD3δ, TCRα, TCRβ, TCRζ, CD32, CD64, CD64, CD45, CD5, CD9, CD22, CD37, CD80, CD86, CD40, CD40L/CD154, VEGFR2, FAS, and FGFR2B. In some embodiments, the transmembrane domain is from a membrane protein selected from the following: CD8α, CD8β, 4-1BB/CD137, CD28, CD34, CD4, FcεRIγ, CD16, OX40/CD134, CD3ζ, CD3ε, CD3γ, CD3δ, TCRα, CD32, CD64, VEGFR2, FAS, and FGFR2B. In some examples, the transmembrane domain is of CD8 (e.g., the transmembrane domain is of CD8α). In some examples, the transmembrane domain is of 4-1BB/CD137. In other examples, the transmembrane domain is of CD28. In that case, the ACTR polypeptide described herein may be free of a hinge domain from any non-CD16A receptor. In some instances, such an ACTR polypeptide may be free of any hinge domain. Alternatively or in addition, such an ACTR polypeptide may comprise two or more co-stimulatory regions as described herein. In other examples, the transmembrane domain is of CD34. In yet other examples, the transmembrane domain is not derived from human CD8α. In some embodiments, the transmembrane domain of the ACTR polypeptide is a single-pass alpha helix.

Transmembrane domains from multi-pass membrane proteins may also be compatible for use in the ACTR polypeptides described herein. Multi-pass membrane proteins may comprise a complex alpha helical structure (e.g., at least 2, 3, 4, 5, 6, 7 or more alpha helices) or a beta sheet structure. Preferably, the N-terminus and the C-terminus of a multi-pass membrane protein are present on opposing sides of the lipid bilayer, e.g., the N-terminus of the protein is present on the cytoplasmic side of the lipid bilayer and the C-terminus of the protein is present on the extracellular side. Either one or multiple helix passes from a multi-pass membrane protein can be used for constructing the ACTR polypeptide described herein.

Transmembrane domains for use in the ACTR polypeptides described herein can also comprise at least a portion of a synthetic, non-naturally occurring protein segment. In some embodiments, the transmembrane domain is a synthetic, non-naturally occurring alpha helix or beta sheet. In some embodiments, the protein segment is at least approximately 20 amino acids, e.g., at least 18, 19, 20, 21, 22, 23, 24, 25, 26, 27, 28, 29, 30, or more amino acids. Examples of synthetic transmembrane domains are known in the art, for example in U.S. Pat. No. 7,052,906 B1 and PCT Publication No. WO 2000/032776 A2, the relevant disclosures of which are incorporated by reference herein.

In some embodiments, the amino acid sequence of the transmembrane domain does not comprise cysteine residues. In some embodiments, the amino acid sequence of the transmembrane domain comprises one cysteine residue. In some embodiments, the amino acid sequence of the transmembrane domain comprises two cysteine residues. In some embodiments, the amino acid sequence of the transmembrane domain comprises more than two cysteine residues (e.g., 3, 4, 5, or more).

The transmembrane domain may comprise a transmembrane region and a cytoplasmic region located at the C-terminal side of the transmembrane domain. The cytoplasmic region of the transmembrane domain may comprise three or more amino acids and, in some embodiments, helps to orient the transmembrane domain in the lipid bilayer. In some embodiments, one or more cysteine residues are present in the transmembrane region of the transmembrane domain. In some embodiments, one or more cysteine residues are present in the cytoplasmic region of the transmembrane domain. In some embodiments, the cytoplasmic region of the transmembrane domain comprises positively charged amino acids. In some embodiments, the cytoplasmic region of the transmembrane domain comprises the amino acids arginine, serine, and lysine.

In some embodiments, the transmembrane region of the transmembrane domain comprises hydrophobic amino acid residues. In some embodiments, the transmembrane region comprises mostly hydrophobic amino acid residues, such as alanine, leucine, isoleucine, methionine, phenylalanine, tryptophan, or valine. In some embodiments, the transmembrane region is hydrophobic. In some embodiments, the transmembrane region comprises a poly-leucine-alanine sequence.

The hydropathy, or hydrophobic or hydrophilic characteristics of a protein or protein segment, can be assessed by any method known in the art, for example the Kyte and Doolittle hydropathy analysis.

### C. Co-Stimulatory Signaling Domains

Many immune cells require co-stimulation, in addition to stimulation of an antigen-specific signal, to promote cell proliferation, differentiation and survival, as well as to activate effector functions of the cell. In some embodiments, the ACTR polypeptides described herein comprise at least one co-stimulatory signaling domain. In certain embodiments, the ACTR polypeptides may contain a CD28 co-stimulatory signaling domain. The term “co-stimulatory signaling domain,” as used herein, refers to at least a fragment of a co-stimulatory signaling protein that mediates signal transduction within a cell to induce an immune response such as an effector function. As known in the art, activation of immune cells such as T cells often requires two signals: (1) the antigen specific signal triggered by the engagement of T cell receptor (TCR) and antigenic peptide/MHC complexes presented by antigen presenting cells, which typically is driven by CD3ζ as a component of the TCR complex; and (ii) a co-stimulatory signal triggered by the interaction between a co-stimulatory receptor and its ligand. A co-stimulatory receptor transduces a co-stimulatory signal as an addition to the TCR-triggered signaling and modulates responses mediated by immune cells, such as T cells, NK cells, macrophages, neutrophils, or eosinophils.

Activation of a co-stimulatory signaling domain in a host cell (e.g., an immune cell) may induce the cell to increase or decrease the production and secretion of cytokines, phagocytic properties, proliferation, differentiation, survival, and/or cytotoxicity. The co-stimulatory signaling domain of any co-stimulatory molecule may be compatible for use in the ACTR polypeptides described herein. The type(s) of co-stimulatory signaling domain is selected based on factors such as the type of the immune cells in which the ACTR polypeptides would be expressed (e.g., T cells, NK cells, macrophages, neutrophils, or eosinophils) and the desired immune effector function (e.g., ADCC). Examples of co-stimulatory signaling domains for use in the ACTR polypeptides may be the cytoplasmic signaling domain of co-stimulatory proteins, including, without limitation, members of the B7/CD28 family (e.g., B7-1/CD80, B7-2/CD86, B7-H1/PD-L1, B7-H2, B7-H3, B7-H4, B7-H6, B7-H7, BTLA/CD272, CD28, CTLA-4, Gi24/VISTA/B7-H5, ICOS/CD278, PD-1, PD-L2/B7-DC, and PDCD6); members of the TNF superfamily (e.g., 4-1BB/TNFSF9/CD137, 4-1BB Ligand/TNFSF9, BAFF/BLyS/TNFSF13B, BAFF R/TNFRSF13C, CD27/TNFRSF7, CD27 Ligand/TNFSF7, CD30/TNFRSF8, CD30 Ligand/TNFSF8, CD40/TNFRSF5, CD40/TNFSF5, CD40 Ligand/TNFSF5, DR3/TNFRSF25, GITR/TNFRSF18, GITR Ligand/TNFSF18, HVEM/TNFRSF14, LIGHT/TNFSF14, Lymphotoxin-alpha/TNF-beta, OX40/TNFRSF4, OX40 Ligand/TNFSF4, RELT/TNFRSF19L, TACI/TNFRSF13B, TL1A/TNFSF15, TNF-alpha, and TNF RII/TNFRSF1B); members of the SLAM family (e.g., 2B4/CD244/SLAMF4, BLAME/SLAMF8, CD2, CD2F-10/SLAMF9, CD48/SLAMF2, CD58/LFA-3, CD84/SLAMF5, CD229/SLAMF3, CRACC/SLAMF7, NTB-A/SLAMF6, and SLAM/CD150); and any other co-stimulatory molecules, such as CD2, CD7, CD53, CD82/Kai-1, CD90/Thy1, CD96, CD160, CD200, CD300a/LMIR1, HLA Class I, HLA-DR, Ikaros, Integrin alpha 4/CD49d, Integrin alpha 4 beta 1, Integrin alpha 4 beta 7/LPAM-1, LAG-3, TCL1A, TCL1B, CRTAM, DAP12, Dectin-1/CLEC7A, DPPIV/CD26, EphB6, TIM-1/KIM-1/HAVCR, TIM-4, TSLP, TSLP R, lymphocyte function associated antigen-1 (LFA-1), and NKG2C. In some embodiments, the co-stimulatory signaling domain is of 4-1BB, CD28, OX40, ICOS, CD27, GITR, HVEM, TIM1, LFA1(CD11a) or CD2, or any variant thereof.

Also within the scope of the present disclosure are variants of any of the co-stimulatory signaling domains described herein, such that the co-stimulatory signaling domain is capable of modulating the immune response of the immune cell. In some embodiments, the co-stimulatory signaling domains comprises up to 10 amino acid residue mutations (e.g., 1, 2, 3, 4, 5, or 8) such as amino acid substitutions, deletions, or additions as compared to a wild-type counterpart. Such co-stimulatory signaling domains comprising one or more amino acid variations (e.g., amino acid substitutions, deletions, or additions) may be referred to as variants.

Mutation of amino acid residues of the co-stimulatory signaling domain may result in an increase in signaling transduction and enhanced stimulation of immune responses relative to co-stimulatory signaling domains that do not comprise the mutation. Mutation of amino acid residues of the co-stimulatory signaling domain may result in a decrease in signaling transduction and reduced stimulation of immune responses relative to co-stimulatory signaling domains that do not comprise the mutation. For example, mutation of residues 186 and 187 of the native CD28 amino acid sequence may result in an increase in co-stimulatory activity and induction of immune responses by the co-stimulatory domain of the ACTR polypeptide. In some embodiments, the mutations are substitution of a lysine at each of positions 186 and 187 with a glycine residue of the CD28 co-stimulatory domain, referred to as a CD28_{LL→GG} variant. Additional mutations that can be made in co-stimulatory signaling domains that may enhance or reduce co-stimulatory activity of the domain will be evident to one of ordinary skill in the art. In some embodiments, the co-stimulatory signaling domain is of 4-1BB, CD28, OX40, or CD28_{LL→GG} variant.

In some embodiments, the ACTR polypeptides may contain a single co-stimulatory domain such as, for example, a CD27 co-stimulatory domain, a CD28 co-stimulatory domain, a 4-1BB co-stimulatory domain, an ICOS co-stimulatory domain, or an OX40 co-stimulatory domain.

In some embodiments, the ACTR polypeptides may comprise more than one co-stimulatory signaling domain (e.g., 2, 3, or more). In some embodiments, the ACTR polypeptide comprises two or more of the same co-stimulatory signaling domains, for example, two copies of the co-stimulatory signaling domain of CD28. In some embodiments, the ACTR polypeptide comprises two or more co-stimulatory signaling domains from different co-stimulatory proteins, such as any two or more co-stimulatory proteins described herein. Selection of the type(s) of co-stimulatory signaling domains may be based on factors such as the type of host cells to be used with the ACTR polypeptides (e.g., T cells or NK cells) and the desired immune effector function. In some embodiments, the ACTR polypeptide comprises two co-stimulatory signaling domains, for example, two copies of the co-stimulatory signaling domain of CD28. In some embodiments, the ACTR polypeptide may comprise two or more co-stimulatory signaling domains from different co-stimulatory receptors, such as any two or more co-stimulatory receptors described herein, for example, CD28 and 4-1BB, CD28 and CD27, CD28 and ICOS, CD28_{LL→GG} variant and 4-1BB, CD28 and OX40, or CD28_{LL→GG} variant and OX40. In some embodiments, the two co-stimulatory signaling domains are CD28 and 4-1BB. In some embodiments, the two co-stimulatory signaling domains are CD28_{LL→GG} variant and 4-1BB. In some embodiments, the two co-stimulatory signaling domains are CD28 and OX40. In some embodiments, the two co-stimulatory signaling domains are CD28_{LL→GG} variant and OX40. In some embodiments, the ACTR constructs described herein may contain a combination of a CD28 and ICOSL. In some embodiments, the ACTR construct described herein may contain a combination of CD28 and CD27. In certain embodiments, the 4-1BB co-stimulatory domain is located N-terminal to the CD28 or CD28_{LL→GG} variant co-stimulatory signaling domain.

Any of the ACTR polypeptides described herein, either containing one or more co-stimulatory signaling domain or free of such a signaling domain, may be co-expressed in immune cells (e.g., NK cells or T cells) with one or more separate polypeptides capable of eliciting a co-stimulatory signal in trans, for example, polypeptides comprising a co-stimulatory receptor, a ligand thereof, or a binding moiety (e.g., a single-chain antibody) to a co-stimulatory receptor. As a non-limiting example, the one or more separate polypeptides may comprise 4-1BB ligand (4-1BBL), CD80, CD86, OX40 ligand (OX40L), ICOS ligand (ICOSL), CD70, fragments thereof, or a combination thereof. In some embodiments, the one or more separate polypeptides may comprise 4-1BB, CD28, CD27, CD40L, OX40, ICOS, fragments thereof, a combination thereof. In yet other embodiments, the separate polypeptide may comprise a binding moiety (e.g., a scFv) specific to any of the co-stimulatory receptor or ligand described herein. As a non-limiting example, the one or more separate polypeptides may comprise an scFv that binds to 4-1BB, ICOS, OX40, CD27 or CD28. For example, the one or more separate polypeptides may comprise a scFv from an agonistic anti-4-1BB mAb.

As an example, the amino acid of 4-1BBL is provided below:

4-1BBL sequence
(SEQ ID NO: 24)
MEYASDASLDPEAPWPPAPRARACRVLPWALVAGLLLLLLLAAACAVF
LACPWAVSGARASPGSAASPRLREGPELSPDDPAGLLDLRQGMFAQLV
AQNVLLIDGPLSWYSDPGLAGVSLTGGLSYKEDTKELVVAKAGVYYVF
FQLELRRVVAGEGSGSVSLALHLQPLRSAAGAAALALTVDLPPASSEA
RNSAFGFQGRLLHLSAGQRLGVHLHTEARARHAWQLTQGATVLGLFRV
TPEIPAGLPSPRSE

### D. Cytoplasmic Signaling Domain Comprising an Immunoreceptor Tyrosine-Based Activation Motif (ITAM)

Any cytoplasmic signaling domain comprising an immunoreceptor tyrosine-based activation motif (ITAM) can be used to create the ACTR polypeptides described herein. An “ITAM,” as used herein, is a conserved protein motif that is generally present in the tail portion of signaling molecules expressed in many immune cells. The motif may comprises two repeats of the amino acid sequence YxxL/I separated by 6-8 amino acids, wherein each x is independently any amino acid, producing the conserved motif YxxL/Ix₍₆₋₈₎YxxL/I. ITAMs within signaling molecules are important for signal transduction within the cell, which is mediated at least in part by phosphorylation of tyrosine residues in the ITAM following activation of the signaling molecule. ITAMs may also function as docking sites for other proteins involved in signaling pathways. In some examples, the cytoplasmic signaling domain comprising an ITAM is of CD3ζ or FcεR1γ. In other examples, the ITAM-containing cytoplasmic signaling domain is not derived from human CD3ζ. In yet other examples, the ITAM-containing cytoplasmic signaling domain is not derived from an Fc receptor, when the extracellular ligand-binding domain of the same ACTR polypeptide is derived from CD16A.

In one specific embodiment, several signaling domains can be fused together for additive or synergistic effect. Non-limiting examples of useful additional signaling domains include part or all of one or more of TCR Zeta chain, CD28, OX40/CD134, 4-1BB/CD137, FcεRIγ, ICOS/CD278, ILRB/CD122, IL-2RG/CD132, and CD40.

### E. Hinge Domain

In some embodiments, the ACTR polypeptides described herein further comprise a hinge domain that is located between the extracellular ligand-binding domain and the transmembrane domain. A hinge domain is an amino acid segment that is generally found between two domains of a protein and may allow for flexibility of the protein and movement of one or both of the domains relative to one another. Any amino acid sequence that provides such flexibility and movement of the extracellular ligand-binding domain of an Fc receptor relative to the transmembrane domain of the ACTR polypeptide can be used.

The hinge domain may contain about 1-100 amino acids, e.g., about 1-60 amino acids (including 1-30 amino acids or 31-60 amino acids) or about 50-100 amino acids (including 51-75 amino acids or 76-100 amino acids). As a non-limiting example, the hinge may be 1-15 amino acids, 15-75 amino acids, 20-50 amino acids, or 30-60 amino acids. In some embodiments, the hinge domain may be of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, or 75 amino acids in length. In some embodiments, an ACTR construct described herein contains no hinge domain.

The term “about” or “approximately” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value. In some embodiments, the hinge domain is a hinge domain of a naturally occurring protein.

Hinge domains of any protein known in the art to comprise a hinge domain are compatible for use in the ACTR polypeptides described herein. In some embodiments, the hinge domain is at least a portion of a hinge domain of a naturally occurring protein and confers flexibility to the ACTR polypeptide. In some embodiments, the hinge domain is of CD8. In some embodiments, the hinge domain is a portion of the hinge domain of CD8, e.g., a fragment containing at least 15 (e.g., 20, 25, 30, 35, or 40) consecutive amino acids of the hinge domain of CD8. In some embodiments, the hinge domain is of CD28. In some embodiments, the hinge domain is a portion of the hinge domain of CD28, e.g., a fragment containing at least 15 (e.g., 20, 25, 30, 35, or 40) consecutive amino acids of the hinge domain of CD28.

In some embodiments, the hinge domain is of CD16A receptor, for example, the whole hinge domain of a CD16A receptor or a portion thereof, which may consists of up to 40 consecutive amino acid residues of the CD16A receptor (e.g., 20, 25, 30, 35, or 40). Such an ACTR construct may contain no hinge domain from a different receptor (a non-CD16A receptor).

Hinge domains of antibodies, such as an IgG, IgA, IgM, IgE, or IgD antibodies, are also compatible for use in the ACTR polypeptides described herein. In some embodiments, the hinge domain is the hinge domain that joins the constant domains CH1 and CH2 of an antibody. In some embodiments, the hinge domain is of an antibody and comprises the hinge domain of the antibody and one or more constant regions of the antibody. In some embodiments, the hinge domain comprises the hinge domain of an antibody and the CH3 constant region of the antibody. In some embodiments, the hinge domain comprises the hinge domain of an antibody and the CH2 and CH3 constant regions of the antibody. In some embodiments, the antibody is an IgG, IgA, IgM, IgE, or IgD antibody. In some embodiments, the antibody is an IgG antibody. In some embodiments, the antibody is an IgG1, IgG2, IgG3, or IgG4 antibody. In some embodiments, the hinge region comprises the hinge region and the CH2 and CH3 constant regions of an IgG1 antibody. In some embodiments, the hinge region comprises the hinge region and the CH3 constant region of an IgG1 antibody.

Non-naturally occurring peptides may also be used as hinge domains for the ACTR polypeptides described herein. In some embodiments, the hinge domain between the C-terminus of the extracellular ligand-binding domain of an Fc receptor and the N-terminus of the transmembrane domain is a peptide linker, such as a (GlyₓSer)ₙ linker, wherein x and n, independently can be an integer between 3 and 12, including 3, 4, 5, 6, 7, 8, 9, 10, 11, 12, or more. In some embodiments, the hinge domain is (GlyₓSer)ₙ (SEQ ID NO:25), wherein n can be an integer between 3 and 60, including 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60. In certain embodiments, n can be an integer greater than 60. In some embodiments, the hinge domain is (Gly₄Ser)₃ (SEQ ID NO: 28). In some embodiments, the hinge domain is (Gly₄Ser)₆ (SEQ ID NO: 29). In some embodiments, the hinge domain is (Gly₄Ser)₉ (SEQ ID NO: 30). In some embodiments, the hinge domain is (Gly₄Ser)₁₂ (SEQ ID NO: 31). In some embodiments, the hinge domain is (Gly₄Ser)₁₅ (SEQ ID NO: 32). In some embodiments, the hinge domain is (Gly₄Ser)₃₀ (SEQ ID NO: 33). In some embodiments, the hinge domain is (Gly₄Ser)₄₅ (SEQ ID NO: 34). In some embodiments, the hinge domain is (Gly₄Ser)₆₀ (SEQ ID NO: 35).

In other embodiments, the hinge domain is an extended recombinant polypeptide (XTEN), which is an unstructured polypeptide consisting of hydrophilic residues of varying lengths (e.g., 10-80 amino acid residues). Amino acid sequences of XTEN peptides will be evident to one of skill in the art and can be found, for example, in U.S. Pat. No. 8,673,860, the relevant disclosures of which are incorporated by reference herein. In some embodiments, the hinge domain is an XTEN peptide and comprises 60 amino acids. In some embodiments, the hinge domain is an XTEN peptide and comprises 30 amino acids. In some embodiments, the hinge domain is an XTEN peptide and comprises 45 amino acids. In some embodiments, the hinge domain is an XTEN peptide and comprises 15 amino acids.

### F. Signal Peptide

In some embodiments, the ACTR polypeptide also comprises a signal peptide (also known as a signal sequence) at the N-terminus of the polypeptide. In general, signal sequences are peptide sequences that target a polypeptide to the desired site in a cell. In some embodiments, the signal sequence targets the ACTR polypeptide to the secretory pathway of the cell and will allow for integration and anchoring of the ACTR polypeptide into the lipid bilayer. Signal sequences including signal sequences of naturally occurring proteins or synthetic, non-naturally occurring signal sequences that are compatible for use in the ACTR polypeptides described herein will be evident to one of skill in the art. In some embodiments, the signal sequence from CD8α. In some embodiments, the signal sequence is from CD28. In other embodiments, the signal sequence is from the murine kappa chain. In yet other embodiments, the signal sequence is from CD16.

### G. Examples of ACTR Polypeptides

Certain examples of ACTR polypeptides described herein may have, e.g., a CD16A Fc binding domain, a CD28 co-stimulatory domain, and a CD3ζ cytoplasmic signaling domain. Such ACTR polypeptides may further comprise a CD28 hinge domain, a CD28 transmembrane domain, or a combination thereof. In some examples, the ACTR polypeptide may further comprise a signal sequence, which may be from CD8α. In one specific example, the ACTR polypeptide comprises, from N-terminus to C-terminus in order: a signal sequence of CD8α, a CD16A Fc binding domain, a CD28 hinge domain, a CD28 transmembrane domain, a CD28 co-stimulatory domain, and a CD3ζ cytoplasmic signaling domain, e.g., SEQ ID NO: 9. In some embodiments, any of the ACTR polypeptides described above (e.g., SEQ ID NO:9) may be combined with a separate polypeptide that provides a co-stimulatory signaling as described herein, for example, comprising a 4-1BBL domain (e.g., SEQ ID NO:39).

Other examples of ACTR polypeptides described herein may be free of a hinge domain from any non-CD16A receptor (e.g., may have no hinge domain). Such ACTR polypeptides may have, e.g., a CD16A Fc binding domain, a CD28 co-stimulatory domain, and a CD3ζ cytoplasmic signaling domain, but have no hinge domain. In some examples, the ACTR polypeptide may additionally comprise a CD8 transmembrane domain. In some examples, the ACTR polypeptide may further comprise a signal sequence, which may be from CD8α. In one specific example, the ACTR polypeptide comprises, from N-terminus to C-terminus in order: a signal sequence of CD8α, a CD16A Fc binding domain, a CD8 transmembrane domain, a CD28 co-stimulatory domain, and a CD3ζ cytoplasmic signaling domain, e.g., SEQ ID NO: 13 or SEQ ID NO: 38. In certain cases, ACTR polypeptides may be expressed with a second polypeptide such as a polypeptide capable of eliciting a co-stimulatory signal. Constructs for expression of ACTR polypeptides with such second polypeptides are described herein.

Table 3 provides exemplary ACTR polypeptides described herein. These exemplary constructs have, from N-terminus to C-terminus in order, the signal sequence, the Fc binding domain (e.g., an extracellular domain of an Fc receptor), the hinge domain, and the transmembrane, while the positions of the optional co-stimulatory domain and the cytoplasmic signaling domain can be switched. In some embodiments, the ACTR polypeptide may comprise any one of SEQ ID NOs: 1-22, 26, 27, 38, and 40. In certain embodiments, the ACTR polypeptide may consist of any one of SEQ ID NOs: 1-22, 26, 27, 38, and 40.

TABLE 3
Exemplary ACTR polypeptides.

| Ex- | | | | | | |
|---|---|---|---|---|---|---|
| emplary | | Extra- | | | | |
| AA | | cellular | | Trans- | | Cyto- |
| Sequence | | domain | | mem- | Co- | plasmic |
| (SEQ ID | Signal | of Fc | Hinge | brane | stimulatory | Signaling |
| NO) | Sequence | receptor | domain | domain | domain(s) | domain |
| 1 | CD8α | CD16A | CD8 | CD8 | CD27 | CD3ζ |
| 2 | CD8α | CD16A | CD8 | CD8 | CD28 | CD3ζ |
| 3 | CD8α | CD16A | CD8 | CD8 | ICOS | CD3ζ |
| 4 | CD8α | CD16A | CD8 | CD8 | OX40 | CD3ζ |
| 5 | CD8α | CD16A | CD8 | CD8 | CD28 and | CD3ζ |
| | | | | | CD27 | |
| 6 | CD8α | CD16A | CD8 | CD8 | CD28 and | CD3ζ |
| | | | | | ICOS | |
| 7 | CD8α | CD16A | CD8 | CD8 | CD28 and | CD3ζ |
| | | | | | OX40 | |
| 8 | CD8α | CD16A | CD8 | CD8 | 4-1BB and | CD3ζ |
| | | | | | CD28 | |
| 9 | CD8α | CD16A | CD28 | CD28 | CD28 | CD3ζ |
| | | | (39 aa) | | | |
| 11 | CD8α | CD16A | none | CD8 | 4-1BB | CD3ζ |
| 12 | CD8α | CD16A | none | CD8 | CD27 | CD3ζ |
| 13 | CD8α | CD16A | none | CD8 | CD28 | CD3ζ |
| 14 | CD8α | CD16A | none | CD8 | ICOS | CD3ζ |
| 15 | CD8α | CD16A | none | CD8 | OX40 | CD3ζ |
| 16 | CD8α | CD16A | none | CD8 + | 4-1BB | CD3ζ |
| | | | | 4aa | | |
| 17 | CD8α | CD16A | none | CD8 + | CD28 | CD3ζ |
| | | | | 4aa | | |
| 19 | CD8α | CD16A | CD8 | CD28 | CD28 | CD3ζ |
| 20 | CD8α | CD16A | CD28 | CD28 | CD28 | CD3ζ |
| | | | (26aa) | | | |
| 21 | CD8α | CD16A | CD28 | CD28 | CD28 | CD3ζ |
| | | | (16aa) | | | |
| 22 | CD8α | CD16A | none | CD28 | CD28 | CD3ζ |
| 26 | CD8α | CD16A | CD8 | CD8 | 41BB | CD3ζ |
| 27 | CD8α | CD16A | CD28 | CD8 | CD28 | CD3ζ |
| | | | (39 aa) | | | |

Amino acid sequences of the example ACTR polypeptides are provided below (signal sequence italicized).

ACTR variant
SEQ ID NO: 1
MALPVTALLLPLALLLHAARPGMRTEDLPKAVVFLEPQWYRVLEKDSV
TLKCQGAYSPEDNSTQWFHNESLISSQASSYFIDAATVDDSGEYRCQT
NLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVN
ITITQGLAVSTISSFFPPGYQTTTPAPRPPTPAPTIASQPLSLRPEAC
RPAAGGAVHTRGLDFACDIYIWAPLAGTCGVLLLSLVITLYCQRRKYR
SNKGESPVEPAEPCHYSCPREEEGSTIPIQEDYRKPEPACSPRVKFSR
SADAPAYQQGQNQLYNELNLGRREEYDVLDKRRGRDPEMGGKPRRKNP
QEGLYNELQKDKMAEAYSEIGMKGERRRGKGHDGLYQGLSTATKDTYD
ALHMQALPPR
ACTR variant
SEQ ID NO: 2
MALPVTALLLPLALLLHAARPGMRTEDLPKAVVFLEPQWYRVLEKDSV
TLKCQGAYSPEDNSTQWFHNESLISSQASSYFIDAATVDDSGEYRCQT
NLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVN
ITITQGLAVSTISSFFPPGYQTTTPAPRPPTPAPTIASQPLSLRPEAC
RPAAGGAVHTRGLDFACDIYIWAPLAGTCGVLLLSLVITLYCRSKRSR
LLHSDYMNMTPRRPGPTRKHYQPYAPPRDFAAYRSRVKFSRSADAPAY
QQGQNQLYNELNLGRREEYDVLDKRRGRDPEMGGKPRRKNPQEGLYNE
LQKDKMAEAYSEIGMKGERRRGKGHDGLYQGLSTATKDTYDALHMQAL
PPR
ACTR variant
SEQ ID NO: 3
MALPVTALLLPLALLLHAARPGMRTEDLPKAVVFLEPQWYRVLEKDSV
TLKCQGAYSPEDNSTQWFHNESLISSQASSYFIDAATVDDSGEYRCQT
NLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVN
ITITQGLAVSTISSFFPPGYQTTTPAPRPPTPAPTIASQPLSLRPEAC
RPAAGGAVHTRGLDFACDIYIWAPLAGTCGVLLLSLVITLYCKKKYSS
SVHDPNGEYMFMRAVNTAKKSRLTDVTLRVKFSRSADAPAYQQGQNQL
YNELNLGRREEYDVLDKRRGRDPEMGGKPRRKNPQEGLYNELQKDKMA
EAYSEIGMKGERRRGKGHDGLYQGLSTATKDTYDALHMQALPPR
ACTR variant
SEQ ID NO: 4
MALPVTALLLPLALLLHAARPGMRTEDLPKAVVFLEPQWYRVLEKDSV
TLKCQGAYSPEDNSTQWFHNESLISSQASSYFIDAATVDDSGEYRCQT
NLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVN
ITITQGLAVSTISSFFPPGYQTTTPAPRPPTPAPTIASQPLSLRPEAC
RPAAGGAVHTRGLDFACDIYIWAPLAGTCGVLLLSLVITLYCRRDQRL
PPDAHKPPGGGSFRTPIQEEQADAHSTLAKIRVKFSRSADAPAYQQGQ
NQLYNELNLGRREEYDVLDKRRGRDPEMGGKPRRKNPQEGLYNELQKD
KMAEAYSEIGMKGERRRGKGHDGLYQGLSTATKDTYDALHMQALPPR
ACTR variant
SEQ ID NO: 5
MALPVTALLLPLALLLHAARPGMRTEDLPKAVVFLEPQWYRVLEKDSV
TLKCQGAYSPEDNSTQWFHNESLISSQASSYFIDAATVDDSGEYRCQT
NLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVN
ITITQGLAVSTISSFFPPGYQTTTPAPRPPTPAPTIASQPLSLRPEAC
RPAAGGAVHTRGLDFACDIYIWAPLAGTCGVLLLSLVITLYCRSKRSR
LLHSDYMNMTPRRPGPTRKHYQPYAPPRDFAAYRSQRRKYRSNKGESP
VEPAEPCHYSCPREEEGSTIPIQEDYRKPEPACSPRVKFSRSADAPAY
QQGQNQLYNELNLGRREEYDVLDKRRGRDPEMGGKPRRKNPQEGLYNE
LQKDKMAEAYSEIGMKGERRRGKGHDGLYQGLSTATKDTYDALHMQAL
PPR
ACTR variant
SEQ ID NO: 6
MALPVTALLLPLALLLHAARPGMRTEDLPKAVVFLEPQWYRVLEKDSV
TLKCQGAYSPEDNSTQWFHNESLISSQASSYFIDAATVDDSGEYRCQT
NLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVN
ITITQGLAVSTISSFFPPGYQTTTPAPRPPTPAPTIASQPLSLRPEAC
RPAAGGAVHTRGLDFACDIYIWAPLAGTCGVLLLSLVITLYCRSKRSR
LLHSDYMNMTPRRPGPTRKHYQPYAPPRDFAAYRSKKKYSSSVHDPNG
EYMFMRAVNTAKKSRLTDVTLRVKFSRSADAPAYQQGQNQLYNELNLG
RREEYDVLDKRRGRDPEMGGKPRRKNPQEGLYNELQKDKMAEAYSEIG
MKGERRRGKGHDGLYQGLSTATKDTYDALHMQALPPR
ACTR variant
SEQ ID NO: 7
MALPVTALLLPLALLLHAARPGMRTEDLPKAVVFLEPQWYRVLEKDSV
TLKCQGAYSPEDNSTQWFHNESLISSQASSYFIDAATVDDSGEYRCQT
NLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVN
ITITQGLAVSTISSFFPPGYQTTTPAPRPPTPAPTIASQPLSLRPEAC
RPAAGGAVHTRGLDFACDIYIWAPLAGTCGVLLLSLVITLYCRSKRSR
LLHSDYMNMTPRRPGPTRKHYQPYAPPRDFAAYRSRRDQRLPPDAHKP
PGGGSFRTPIQEEQADAHSTLAKIRVKFSRSADAPAYQQGQNQLYNEL
NLGRREEYDVLDKRRGRDPEMGGKPRRKNPQEGLYNELQKDKMAEAYS
EIGMKGERRRGKGHDGLYQGLSTATKDTYDALHMQALPPR
ACTR variant
SEQ ID NO: 8
MALPVTALLLPLALLLHAARPGMRTEDLPKAVVFLEPQWYRVLEKDSV
TLKCQGAYSPEDNSTQWFHNESLISSQASSYFIDAATVDDSGEYRCQT
NLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVN
ITITQGLAVSTISSFFPPGYQTTTPAPRPPTPAPTIASQPLSLRPEAC
RPAAGGAVHTRGLDFACDIYIWAPLAGTCGVLLLSLVITLYCKRGRKK
LLYIFKQPFMRPVQTTQEEDGCSCRFPEEEEGGCELRSKRSRLLHSDY
MNMTPRRPGPTRKHYQPYAPPRDFAAYRSRVKFSRSADAPAYQQGQNQ
LYNELNLGRREEYDVLDKRRGRDPEMGGKPRRKNPQEGLYNELQKDKM
AEAYSEIGMKGERRRGKGHDGLYQGLSTATKDTYDALHMQALPPR
ACTR variant
SEQ ID NO: 9
MALPVTALLLPLALLLHAARPGMRTEDLPKAVVFLEPQWYRVLEKDSV
TLKCQGAYSPEDNSTQWFHNESLISSQASSYFIDAATVDDSGEYRCQT
NLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVN
ITITQGLAVSTISSFFPPGYQIEVMYPPPYLDNEKSNGTIIHVKGKHL
CPSPLFPGPSKPFWVLVVVGGVLACYSLLVTVAFIIFWVRSKRSRLLH
SDYMNMTPRRPGPTRKHYQPYAPPRDFAAYRSRVKFSRSADAPAYQQG
QNQLYNELNLGRREEYDVLDKRRGRDPEMGGKPRRKNPQEGLYNELQK
DKMAEAYSEIGMKGERRRGKGHDGLYQGLSTATKDTYDALHMQALPPR
ACTR variant
SEQ ID NO: 11
MALPVTALLLPLALLLHAARPGMRTEDLPKAVVFLEPQWYRVLEKDSV
TLKCQGAYSPEDNSTQWFHNESLISSQASSYFIDAATVDDSGEYRCQT
NLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVN
ITITQGLAVSTISSFFPPGYQIYIWAPLAGTCGVLLLSLVITLYCKRG
RKKLLYIFKQPFMRPVQTTQEEDGCSCRFPEEEEGGCELRVKFSRSAD
APAYQQGQNQLYNELNLGRREEYDVLDKRRGRDPEMGGKPRRKNPQEG
LYNELQKDKMAEAYSEIGMKGERRRGKGHDGLYQGLSTATKDTYDALH
MQALPPR
ACTR variant
SEQ ID NO: 12
MALPVTALLLPLALLLHAARPGMRTEDLPKAVVFLEPQWYRVLEKDSV
TLKCQGAYSPEDNSTQWFHNESLISSQASSYFIDAATVDDSGEYRCQT
NLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVN
ITITQGLAVSTISSFFPPGYQIYIWAPLAGTCGVLLLSLVITLYCQRR
KYRSNKGESPVEPAEPCHYSCPREEEGSTIPIQEDYRKPEPACSPRVK
FSRSADAPAYQQGQNQLYNELNLGRREEYDVLDKRRGRDPEMGGKPRR
KNPQEGLYNELQKDKMAEAYSEIGMKGERRRGKGHDGLYQGLSTATKD
TYDALHMQALPPR
ACTR variant
SEQ ID NO: 13
MALPVTALLLPLALLLHAARPGMRTEDLPKAVVFLEPQWYRVLEKDSV
TLKCQGAYSPEDNSTQWFHNESLISSQASSYFIDAATVDDSGEYRCQT
NLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVN
ITITQGLAVSTISSFFPPGYQIYIWAPLAGTCGVLLLSLVITLYCRSK
RSRLLHSDYMNMTPRRPGPTRKHYQPYAPPRDFAAYRSRVKFSRSADA
PAYQQGQNQLYNELNLGRREEYDVLDKRRGRDPEMGGKPRRKNPQEGL
YNELQKDKMAEAYSEIGMKGERRRGKGHDGLYQGLSTATKDTYDALHM
QALPPR
ACTR variant
SEQ ID NO: 14
MALPVTALLLPLALLLHAARPGMRTEDLPKAVVFLEPQWYRVLEKDSV
TLKCQGAYSPEDNSTQWFHNESLISSQASSYFIDAATVDDSGEYRCQT
NLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVN
ITITQGLAVSTISSFFPPGYQIYIWAPLAGTCGVLLLSLVITLYCKKK
YSSSVHDPNGEYMFMRAVNTAKKSRLTDVTLRVKFSRSADAPAYQQGQ
NQLYNELNLGRREEYDVLDKRRGRDPEMGGKPRRKNPQEGLYNELQKD
KMAEAYSEIGMKGERRRGKGHDGLYQGLSTATKDTYDALHMQALPPR
ACTR variant
SEQ ID NO: 15
MALPVTALLLPLALLLHAARPGMRTEDLPKAVVFLEPQWYRVLEKDSV
TLKCQGAYSPEDNSTQWFHNESLISSQASSYFIDAATVDDSGEYRCQT
NLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVN
ITITQGLAVSTISSFFPPGYQIYIWAPLAGTCGVLLLSLVITLYCRRD
QRLPPDAHKPPGGGSFRTPIQEEQADAHSTLAKIRVKFSRSADAPAYQ
QGQNQLYNELNLGRREEYDVLDKRRGRDPEMGGKPRRKNPQEGLYNEL
QKDKMAEAYSEIGMKGERRRGKGHDGLYQGLSTATKDTYDALHMQALP
PR
ACTR variant
SEQ ID NO: 16
MALPVTALLLPLALLLHAARPGMRTEDLPKAVVFLEPQWYRVLEKDSV
TLKCQGAYSPEDNSTQWFHNESLISSQASSYFIDAATVDDSGEYRCQT
NLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVN
ITITQGLAVSTISSFFPPGYQFACDIYIWAPLAGTCGVLLLSLVITLY
CKRGRKKLLYIFKQPFMRPVQTTQEEDGCSCRFPEEEEGGCELRVKFS
RSADAPAYQQGQNQLYNELNLGRREEYDVLDKRRGRDPEMGGKPRRKN
PQEGLYNELQKDKMAEAYSEIGMKGERRRGKGHDGLYQGLSTATKDTY
DALHMQALPPR
ACTR variant
SEQ ID NO: 17
MALPVTALLLPLALLLHAARPGMRTEDLPKAVVFLEPQWYRVLEKDSV
TLKCQGAYSPEDNSTQWFHNESLISSQASSYFIDAATVDDSGEYRCQT
NLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVN
ITITQGLAVSTISSFFPPGYQFACDIYIWAPLAGTCGVLLLSLVITLY
CRSKRSRLLHSDYMNMTPRRPGPTRKHYQPYAPPRDFAAYRSRVKFSR
SADAPAYQQGQNQLYNELNLGRREEYDVLDKRRGRDPEMGGKPRRKNP
QEGLYNELQKDKMAEAYSEIGMKGERRRGKGHDGLYQGLSTATKDTYD
ALHMQALPPR
ACTR variant
SEQ ID NO: 19
MALPVTALLLPLALLLHAARPGMRTEDLPKAVVFLEPQWYRVLEKDSV
TLKCQGAYSPEDNSTQWFHNESLISSQASSYFIDAATVDDSGEYRCQT
NLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVN
ITITQGLAVSTISSFFPPGYQTTTPAPRPPTPAPTIASQPLSLRPEAC
RPAAGGAVHTRGLDFACDFWVLVVVGGVLACYSLLVTVAFIIFWVRSK
RSRLLHSDYMNMTPRRPGPTRKHYQPYAPPRDFAAYRSRVKFSRSADA
PAYQQGQNQLYNELNLGRREEYDVLDKRRGRDPEMGGKPRRKNPQEGL
YNELQKDKMAEAYSEIGMKGERRRGKGHDGLYQGLSTATKDTYDALHM
QALPPR
ACTR variant
SEQ ID NO: 20
MALPVTALLLPLALLLHAARPGMRTEDLPKAVVFLEPQWYRVLEKDSV
TLKCQGAYSPEDNSTQWFHNESLISSQASSYFIDAATVDDSGEYRCQT
NLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVN
ITITQGLAVSTISSFFPPGYQKSNGTIIHVKGKHLCPSPLFPGPSKPF
WVLVVVGGVLACYSLLVTVAFIIFWVRSKRSRLLHSDYMNMTPRRPGP
TRKHYQPYAPPRDFAAYRSRVKFSRSADAPAYQQGQNQLYNELNLGRR
EEYDVLDKRRGRDPEMGGKPRRKNPQEGLYNELQKDKMAEAYSEIGMK
GERRRGKGHDGLYQGLSTATKDTYDALHMQALPPR
ACTR variant
SEQ ID NO: 21
MALPVTALLLPLALLLHAARPGMRTEDLPKAVVFLEPQWYRVLEKDSV
TLKCQGAYSPEDNSTQWFHNESLISSQASSYFIDAATVDDSGEYRCQT
NLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVN
ITITQGLAVSTISSFFPPGYQGKHLCPSPLFPGPSKPFWVLVVVGGVL
ACYSLLVTVAFIIFWVRSKRSRLLHSDYMNMTPRRPGPTRKHYQPYAP
PRDFAAYRSRVKFSRSADAPAYQQGQNQLYNELNLGRREEYDVLDKRR
GRDPEMGGKPRRKNPQEGLYNELQKDKMAEAYSEIGMKGERRRGKGHD
GLYQGLSTATKDTYDALHMQALPPR
ACTR variant
SEQ ID NO: 22
MALPVTALLLPLALLLHAARPGMRTEDLPKAVVFLEPQWYRVLEKDSV
TLKCQGAYSPEDNSTQWFHNESLISSQASSYFIDAATVDDSGEYRCQT
NLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVN
ITITQGLAVSTISSFFPPGYQFWVLVVVGGVLACYSLLVTVAFIIFWV
RSKRSRLLHSDYMNMTPRRPGPTRKHYQPYAPPRDFAAYRSRVKFSRS
ADAPAYQQGQNQLYNELNLGRREEYDVLDKRRGRDPEMGGKPRRKNPQ
EGLYNELQKDKMAEAYSEIGMKGERRRGKGHDGLYQGLSTATKDTYDA
LHMQALPPR
ACTR variant
SEQ ID NO: 26
MALPVTALLLPLALLLHAARPGMRTEDLPKAVVFLEPQWYRVLEKDSV
TLKCQGAYSPEDNSTQWFHNESLISSQASSYFIDAATVDDSGEYRCQT
NLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVN
ITITQGLAVSTISSFFPPGYQTTTPAPRPPTPAPTIASQPLSLRPEAC
RPAAGGAVHTRGLDFACDIYIWAPLAGTCGVLLLSLVITLYCKRGRKK
LLYIFKQPFMRPVQTTQEEDGCSCRFPEEEEGGCELRVKFSRSADAPA
YQQGQNQLYNELNLGRREEYDVLDKRRGRDPEMGGKPRRKNPQEGLYN
ELQKDKMAEAYSEIGMKGERRRGKGHDGLYQGLSTATKDTYDALHMQA
LPPR
ACTR variant
SEQ ID NO: 27
MALPVTALLLPLALLLHAARPGMRTEDLPKAVVFLEPQWYRVLEKDSV
TLKCQGAYSPEDNSTQWFHNESLISSQASSYFIDAATVDDSGEYRCQT
NLSTLSDPVQLEVHIGWLLLQAPRWVFKEEDPIHLRCHSWKNTALHKV
TYLQNGKGRKYFHHNSDFYIPKATLKDSGSYFCRGLVGSKNVSSETVN
ITITQGLAVSTISSFFPPGYQIEVMYPPPYLDNEKSNGTIIHVKGKHL
CPSPLFPGPSKPIYIWAPLAGTCGVLLLSLVITLYCRSKRSRLLHSDY
MNMTPRRPGPTRKHYQPYAPPRDFAAYRSRVKFSRSADAPAYQQGQNQ
LYNELNLGRREEYDVLDKRRGRDPEMGGKPRRKNPQEGLYNELQKDKM
AEAYSEIGMKGERRRGKGHDGLYQGLSTATKDTYDALHMQALPPR

Further provided below are exemplary ACTR constructs in trans form (containing an ACTR polypeptide and a separate polypeptide providing co-stimulatory signaling in trans).

ACTR variant SEQ ID NO: 38/SEQ ID NO: 39:
ACTR Polypeptide:
(SEQ ID NO: 38)
MALPVTALLLPLALLLHAARPGMRTEDLPKAVVFLEPQWYRVLEKDSVTLK
CQGAYSPEDNSTQWFHNESLISSQASSYFIDAATVDDSGEYRCQTNLSTLS
DPVQLEVHIGWLLLQAPRWVFKEEDPIHLRCHSWKNTALHKVTYLQNGKGR
KYFHHNSDFYIPKATLKDSGSYFCRGLVGSKNVSSETVNITITQGLAVSTI
SSFFPPGYQIYIWAPLAGTCGVLLLSLVITLYCRSKRSRLLHSDYMNMTPR
RPGPTRKHYQPYAPPRDFAAYRSRVKFSRSADAPAYQQGQNQLYNELNLGR
REEYDVLDKRRGRDPEMGGKPRRKNPQEGLYNELQKDKMAEAYSEIGMKGE
RRRGKGHDGLYQGLSTATKDTYDALHMQALPPRGSGATNFSLLKQAGDVEE
NPG
Trans co-stimulator polypeptide (comprising 4-
1BBL):
(SEQ ID NO: 39)
PMEYASDASLDPEAPWPPAPRARACRVLPWALVAGLLLLLLLAAACAVFLA
CPWAVSGARASPGSAASPRLREGPELSPDDPAGLLDLRQGMFAQLVAQNVL
LIDGPLSWYSDPGLAGVSLTGGLSYKEDTKELVVAKAGVYYVFFQLELRRV
VAGEGSGSVSLALHLQPLRSAAGAAALALTVDLPPASSEARNSAFGFQGRL
LHLSAGQRLGVHLHTEARARHAWQLTQGATVLGLFRVTPEIPAGLPSPRSE
ACTR variant SEQ ID NO: 40/SEQ ID NO: 39
ACTR polypeptide:
(SEQ ID NO: 40)
MALPVTALLLPLALLLHAARPGMRTEDLPKAVVFLEPQWYRVLEKDSVTLK
CQGAYSPEDNSTQWFHNESLISSQASSYFIDAATVDDSGEYRCQTNLSTLS
DPVQLEVHIGWLLLQAPRWVFKEEDPIHLRCHSWKNTALHKVTYLQNGKGR
KYFHHNSDFYIPKATLKDSGSYFCRGLVGSKNVSSETVNITITQGLAVSTI
SSFFPPGYQTTTPAPRPPTPAPTIASQPLSLRPEACRPAAGGAVHTRGLDF
ACDIYIWAPLAGTCGVLLLSLVITLYCRSKRSRLLHSDYMNMTPRRPGPTR
KHYQPYAPPRDFAAYRSRVKFSRSADAPAYQQGQNQLYNELNLGRREEYDV
LDKRRGRDPEMGGKPRRKNPQEGLYNELQKDKMAEAYSEIGMKGERRRGKG
HDGLYQGLSTATKDTYDALHMQALPPRGSGATNFSLLKQAGDVEENPG

Trans co-stimulator polypeptide is SEQ ID NO: 39 is provided above.

An explanation of the italicized domains and underlined domains in SEQ ID NOs:38, 39, and 40 is provided below in connection with SEQ ID NO:18 and SEQ ID NO:10.

### H. Nucleic Acids Encoding ACTR Constructs

The present disclosure also provides polynucleotides encoding the ACTR receptors disclosed herein. In conjunction with the polynucleotides, the present disclosure also provides vectors comprising such polynucleotides (including vectors in which such polynucleotides are operatively linked to at least one regulatory element for expression of a chimeric receptor). Non-limiting examples of useful vectors of the disclosure include viral vectors such as, e.g., retroviral vectors including gamma retroviral vectors, adeno-associated virus vectors (AAV vectors), and lentiviral vectors.

In some instances, the nucleic acid described herein may comprise two coding sequences, one encoding an ACTR polypeptide as described herein, and the other encoding a polypeptide capable of eliciting a co-stimulatory signal. Such a polypeptide may comprise a co-stimulatory domain from a co-stimulatory receptor such as 4-1BB, CD28, OX40, CD27, ICOS, or a combination thereof. Alternatively or in addition, the polypeptide may comprise a ligand of a co-stimulatory receptor, for example, 4-1BB ligand (4-1BBL), CD80, CD86, OX40 ligand (OX40L), ICOS ligand (ICOSL), CD70, a functional fragment thereof, or a combination thereof.

The nucleic acid comprising the two coding sequences described herein may be configured such that the polypeptides encoded by the two coding sequences can be expressed as independent (and physically separate) polypeptides. To achieve this goal, the nucleic acid described herein may contain a third nucleotide sequence located between the first and second coding sequences. This third nucleotide sequence may, for example, encode a ribosomal skipping site. A ribosomal skipping site is a sequence that impairs normal peptide bond formation. This mechanism results in the translation of additional open reading frames from one messenger RNA. This third nucleotide sequence may, for example, encode a P2A, T2A, or F2A peptide (see, for example, Kim et al., PLoS One. 2011; 6(4):e18556). As a non-limiting example, an exemplary P2A peptide may have the amino acid sequence of ATNFSLLKQAGDVEENPGP SEQ ID NO.: 23.

In another embodiment, the third nucleotide sequence may encode an internal ribosome entry site (IRES). An IRES is a RNA element that allows translation initiation in an end-independent manner, also permitting the translation of additional open reading frames from one messenger RNA.

In another embodiment, the third nucleotide sequence may encode a second promoter controlling the expression of the second polypeptide.

The third nucleotide sequence may also encode more than one ribosomal skipping sequence, IRES sequence, additional promoter sequence, or a combination thereof.

The nucleic acid may also include additional coding sequences (including, but not limited to, fourth and fifth coding sequences) and may be configured such that the polypeptides encoded by the additional coding sequences are expressed as further independent and physically separate polypeptides. To this end, the additional coding sequences may be separated from other coding sequences by one or more nucleotide sequences encoding one or more ribosomal skipping sequences, IRES sequences, or additional promoter sequences.

In some examples, the nucleic acids described herein may encode an ACTR polypeptide and a second polypeptide capable of eliciting a co-stimulatory signal, which are linked by a P2A peptide. During expression, the ACTR and the second polypeptide would be produced as independent and physically separate polypeptides due to the presence of the P2A site. As a set of non-limiting examples, the expression of nucleotide sequences encoding SEQ ID NO: 10 and SEQ ID NO: 18, both carrying a P2A ribosomal skipping site, would produce two physically separate proteins: one comprising an ACTR protein (SEQ ID NO: 40 and SEQ ID NO: 38, respectively) and the other comprising 4-1BB ligand (4-1BBL) protein (SEQ ID NO: 39). In one embodiment, the two proteins produced by the expression of a nucleotide sequence encoding SEQ ID NO: 18 are shown as SEQ ID NO: 38 and SEQ ID NO: 39. In another embodiment, the two proteins produced by the expression of a nucleotide sequence encoding SEQ ID NO: 10 are shown as SEQ ID NO: 40 and SEQ ID NO: 39.

ACTR sequence + linker + P2A + 4-1BBL
(SEQ ID NO: 10)
MALPVTALLLPLALLLHAARPGMRTEDLPKAVVFLEPQWYRVLEKDSVTLK
CQGAYSPEDNSTQWFHNESLISSQASSYFIDAATVDDSGEYRCQTNLSTLS
DPVQLEVHIGWLLLQAPRWVFKEEDPIHLRCHSWKNTALHKVTYLQNGKGR
KYFHHNSDFYIPKATLKDSGSYFCRGLVGSKNVSSETVNITITQGLAVSTI
SSFFPPGYQTTTPAPRPPTPAPTIASQPLSLRPEACRPAAGGAVHTRGLDF
ACDIYIWAPLAGTCGVLLLSLVITLYCRSKRSRLLHSDYMNMTPRRPGPTR
KHYQPYAPPRDFAAYRSRVKFSRSADAPAYQQGQNQLYNELNLGRREEYDV
LDKRRGRDPEMGGKPRRKNPQEGLYNELQKDKMAEAYSEIGMKGERRRGKG
HDGLYQGLSTATKDTYDALHMQALPPRGSGATNFSLLKQAGDVEENPGPME
YASDASLDPEAPWPPAPRARACRVLPWALVAGLLLLLLLAAACAVFLACPW
AVSGARASPGSAASPRLREGPELSPDDPAGLLDLRQGMFAQLVAQNVLLID
GPLSWYSDPGLAGVSLTGGLSYKEDTKELVVAKAGVYYVFFQLELRRVVAG
EGSGSVSLALHLQPLRSAAGAAALALTVDLPPASSEARNSAFGFQGRLLHL
SAGQRLGVHLHTEARARHAWQLTQGATVLGLFRVTPEIPAGLPSPRSE
ACTR sequence + linker + P2A + 4-1BBL
(SEQ ID NO: 18)
MALPVTALLLPLALLLHAARPGMRTEDLPKAVVFLEPQWYRVLEKDSVTLK
CQGAYSPEDNSTQWFHNESLISSQASSYFIDAATVDDSGEYRCQTNLSTLS
DPVQLEVHIGWLLLQAPRWVFKEEDPIHLRCHSWKNTALHKVTYLQNGKGR
KYFHHNSDFYIPKATLKDSGSYFCRGLVGSKNVSSETVNITITQGLAVSTI
SSFFPPGYQIYIWAPLAGTCGVLLLSLVITLYCRSKRSRLLHSDYMNMTPR
RPGPTRKHYQPYAPPRDFAAYRSRVKFSRSADAPAYQQGQNQLYNELNLGR
REEYDVLDKRRGRDPEMGGKPRRKNPQEGLYNELQKDKMAEAYSEIGMKGE
RRRGKGHDGLYQGLSTATKDTYDALHMQALPPRGSGATNFSLLKQAGDVEE
NPGPMEYASDASLDPEAPWPPAPRARACRVLPWALVAGLLLLLLLAAACAV
FLACPWAVSGARASPGSAASPRLREGPELSPDDPAGLLDLRQGMFAQLVAQ
NVLLIDGPLSWYSDPGLAGVSLTGGLSYKEDTKELVVAKAGVYYVFFQLEL
RRVVAGEGSGSVSLALHLQPLRSAAGAAALALTVDLPPASSEARNSAFGFQ
GRLLHLSAGQRLGVHLHTEARARHAWQLTQGATVLGLFRVTPEIPAGLPSP
RSE

In SEQ ID NOs: 10 and 18 above, the N-terminal italic fragment is the signal peptide, the following domain is the ACTR polypeptide region, the italicized GSG peptide is a linker, the underlined peptide is the P2A ribosomal skipping site, and the C-terminal domain in boldface is the 4-1BBL domain. Upon ribosome skipping, the polypeptide containing the 4-1BBL domain includes an extra “P” residue at the N-terminus and the remaining linker and P2A sequence is attached to the C-terminus of the ACTR polypeptide.

### I. Preparation of and Pharmaceutical Compositions Comprising ACTR Polypeptides

Any of the ACTR polypeptides described herein can be prepared by a routine method, such as recombinant technology. Methods for preparing the ACTR polypeptides herein involve generation of a nucleic acid that encodes a polypeptide comprising each of the domains of the ACTR polypeptides, including the extracellular ligand-binding domain of an Fc receptor, the transmembrane domain, and the cytoplasmic signaling domain comprising an ITAM. The nucleic acid construct may also include one or more co-stimulatory signaling domains. In some embodiments, the nucleic acid also encodes a hinge domain between the extracellular ligand-binding domain of an Fc receptor and the transmembrane domain. The nucleic acid encoding the ACTR polypeptide may also encode a signal sequence. In some embodiments, the nucleic acid sequence encodes any one of the exemplary ACTR polypeptides provided by SEQ ID NO: 1-22, 26, 27, 38, or 40.

Sequences of each of the components of the ACTR polypeptides may be obtained via routine technology, e.g., PCR amplification from any one of a variety of sources known in the art. In some embodiments, sequences of one or more of the components of the ACTR polypeptides are obtained from a human cell. Alternatively, the sequences of one or more components of the ACTR polypeptides can be synthesized. Sequences of each of the components (e.g., domains) can be joined directly or indirectly (e.g., using a nucleic acid sequence encoding a peptide linker) to form a nucleic acid sequence encoding the ACTR polypeptide, using methods such as PCR amplification or ligation. Alternatively, the nucleic acid encoding the ACTR polypeptide may be synthesized. In some embodiments, the nucleic acid is DNA. In other embodiments, the nucleic acid is RNA.

A known limitation of CAR-T cells is the target antigen limitation. When CAR-T cells are specific to T cell antigens, in vitro expansion of such CAR-T cells would be substantially impaired due to fratricide, making it difficult to manufacturing such CAR-T cells in vitro. For example, Dusséaux et al. reported that “expression of CD38 on normal activated T-cells is a significant hurdle for the development of CAR T-cells against this protein.” Dusséaux et al. European Hemalogical Association; June 2016, Poster 365. Further, Gomes-Silva et al. also reported that “expression of a CD7-specific CAR impaired expansion of transduced T cells because of residual CD7 expression and the ensuing fratricide.” Gomes-Silva et al., Blood, 2017, 130: 285-296. Similarly, for CD5 see also Mamonkin et al., Cancer Immunol Res., 2018, 6:47-58. By contrast, the ACTR T cells of the instant invention do not directly target cell surface antigens and thus have no fratricide effect when expanded in vitro.

### II. Immune Cells Expressing ACTR Polypeptides

Genetically engineered host cells (e.g., immune cells such as T cells or NK cells) expressing the ACTR polypeptides (ACTR-expressing cells, e.g., ACTR T cells) described herein provide a specific population of cells that can recognize target cells bound by Fc-containing anti-tumor antibodies. In one embodiment, engagement of the extracellular ligand-binding domain of an ACTR polypeptide expressed on such host cells with the Fc portion of an anti-tumor antibody transmits an activation signal to the optional co-stimulatory signaling domain(s) and/or the ITAM-containing cytoplasmic signaling domain of the ACTR polypeptide, which in turn activates cell proliferation and/or effector functions of the host cell, such as ADCC effects triggered by the host cells. In another embodiment, engagement of the extracellular Fc-binding domain of an ACTR polypeptide expressed on such host cells with the Fc portion of an antibody transmits an activation signal to the ITAM-containing cytoplasmic signaling domain of the ACTR polypeptide and/or the one or more co-stimulatory signaling domains co-expressed in such host cells, which in turn activates cell proliferation and/or effector functions of the host cell, such as ADCC effects triggered by the host cells. The combination of co-stimulatory signaling domain(s) and the cytoplasmic signaling domain comprising an ITAM may allow for robust activation of multiple signaling pathways within the cell. In some embodiments, the host cells are immune cells, such as T cells or NK cells. In some embodiments, the immune cells are T cells. In some embodiments, the immune cells are NK cells. In other embodiments, the immune cells can be established cell lines, for example, NK-92 cells.

Any of the ACTR polypeptides described herein may be co-expressed in the immune cells (e.g., NK cells or T cells) with one or more separate polypeptides described herein for providing co-stimulatory signals in trans, for example, polypeptides comprising one or more signaling domains (e.g., a co-stimulatory domain or a ligand of a co-stimulation factor). As a non-limiting example, the one or more separate polypeptides may comprise 4-1BB ligand (4-1BBL), CD80, CD86, OX40 ligand (OX40L), ICOS ligand (ICOSL), CD70, fragments thereof, or a combination thereof. In one example, the one or more separate polypeptides may encode 4-1BBL.

The population of immune cells can be obtained from any source, such as peripheral blood mononuclear cells (PBMCs), bone marrow, or tissues such as spleen, lymph node, thymus, stem cells, or tumor tissue. A source suitable for obtaining the type of host cells desired would be evident to one of skill in the art. In some embodiments, the population of immune cells is derived from PBMCs. The type of host cells desired (e.g., T cells, NK cells, or T cells and NK cells) may be expanded within the population of cells obtained by co-incubating the cells with stimulatory molecules. As a non-limiting example, anti-CD3 and anti-CD28 antibodies may be used for expansion of T cells.

To construct the immune cells that express any of the ACTR polypeptides described herein, expression vectors for stable or transient expression of the ACTR polypeptide may be created via conventional methods as described herein and introduced into immune host cells. For example, nucleic acids encoding the ACTR polypeptides may be cloned into a suitable expression vector, such as a viral vector in operable linkage to a suitable promoter. The nucleic acids and the vector may be contacted, under suitable conditions, with a restriction enzyme to create complementary ends on each molecule that can pair with each other and be joined with a ligase. Alternatively, synthetic nucleic acid linkers can be ligated to the termini of the nucleic acid encoding the ACTR polypeptides. The synthetic linkers may contain nucleic acid sequences that correspond to a particular restriction site in the vector. The selection of expression vectors/plasmids/viral vectors would depend on the type of host cells for expression of the ACTR polypeptides, but should be suitable for integration and replication in eukaryotic cells.

A variety of promoters can be used for expression of the ACTR polypeptides described herein, including, without limitation, cytomegalovirus (CMV) intermediate early promoter, a viral LTR such as the Rous sarcoma virus LTR, HIV-LTR, HTLV-1 LTR, the simian virus 40 (SV40) early promoter, or herpes simplex tk virus promoter. Additional promoters for expression of the ACTR polypeptides include any constitutively active promoter in an immune cell. Alternatively, any regulatable promoter may be used, such that its expression can be modulated within an immune cell.

Additionally, the vector may contain, for example, some or all of the following: a selectable marker gene, such as the neomycin gene or the kanamycin gene for selection of stable or transient transfectants in host cells; enhancer/promoter sequences from the immediate early gene of human CMV for high levels of transcription; transcription termination and RNA processing signals from SV40 for mRNA stability; SV40 polyomavirus origins of replication and ColE1 for proper episomal replication; internal ribosome binding sites (IRESes), versatile multiple cloning sites; T7 and SP6 RNA promoters for in vitro transcription of sense and antisense RNA; a “suicide switch” or “suicide gene” which when triggered causes cells carrying the vector to die (e.g., HSV thymidine kinase or an inducible caspase such as iCasp9), and reporter gene for assessing expression of the ACTR polypeptide.

In one specific embodiment, such vectors also include a suicide gene. As used herein, the term “suicide gene” refers to a gene that causes the cell expressing the suicide gene to die. The suicide gene can be a gene that confers sensitivity to an agent, e.g., a drug, upon the cell in which the gene is expressed, and causes the cell to die when the cell is contacted with or exposed to the agent. Suicide genes are known in the art (see, for example, Suicide Gene Therapy: Methods and Reviews, Springer, Caroline J. (Cancer Research UK Centre for Cancer Therapeutics at the Institute of Cancer Research, Sutton, Surrey, UK), Humana Press, 2004) and include, for example, the Herpes Simplex Virus (HSV) thymidine kinase (TK) gene, cytosine deaminase, purine nucleoside phosphorylase, nitroreductase, and caspases such as caspase 8.

Suitable vectors and methods for producing vectors containing transgenes are well known and available in the art. Examples of the preparation of vectors for expression of ACTR polypeptides can be found, for example, in US2014/0106449, herein incorporated in its entirety by reference.

Any of the vectors comprising a nucleic acid sequence that encodes an ACTR polypeptide described herein is also within the scope of the present disclosure. Such a vector, or the sequence encoding an ACTR polypeptide contained therein, may be delivered into host cells such as host immune cells by any suitable method. Methods of delivering vectors to immune cells are well known in the art and may include DNA electroporation, RNA electroporation, transfection using reagents such as liposomes, or viral transduction (e.g., retroviral transduction such as lentiviral transduction).

In some embodiments, the vectors for expression of the ACTR polypeptides are delivered to host cells by viral transduction (e.g., retroviral transduction such as lentiviral transduction). Exemplary viral methods for delivery include, but are not limited to, recombinant retroviruses (see, e.g., PCT Publication Nos. WO 90/07936; WO 94/03622; WO 93/25698; WO 93/25234; WO 93/11230; WO 93/10218; and WO 91/02805; U.S. Pat. Nos. 5,219,740 and 4,777,127; GB Patent No. 2,200,651; and EP Patent No. 0 345 242), alphavirus-based vectors, and adeno-associated virus (AAV) vectors (see, e.g., PCT Publication Nos. WO 94/12649, WO 93/03769; WO 93/19191; WO 94/28938; WO 95/11984; and WO 95/00655). In some embodiments, the vectors for expression of the ACTR polypeptides are retroviruses. In some embodiments, the vectors for expression of the ACTR polypeptides are lentiviruses.

Examples of references describing retroviral transduction include Anderson et al., U.S. Pat. No. 5,399,346; Mann et al., Cell 33:153 (1983); Temin et al., U.S. Pat. No. 4,650,764; Temin et al., U.S. Pat. No. 4,980,289; Markowitz et al., J. Virol. 62:1120 (1988); Temin et al., U.S. Pat. No. 5,124,263; International Patent Publication No. WO 95/07358, published Mar. 16, 1995, by Dougherty et al.; and Kuo et al., Blood 82:845 (1993). International Patent Publication No. WO 95/07358 describes high efficiency transduction of primary B lymphocytes. See also WO2016040441A1, which is incorporated by reference herein for the purpose and subject matter referenced herein.

In examples in which the vectors encoding ACTR polypeptides are introduced to the host cells using a viral vector, viral particles that are capable of infecting the immune cells and carry the vector may be produced by any method known in the art and can be found, for example in PCT Application No. WO 1991/002805A2, WO 1998/009271 A1, and U.S. Pat. No. 6,194,191. The viral particles are harvested from the cell culture supernatant and may be isolated and/or purified prior to contacting the viral particles with the immune cells.

In some embodiments, RNA molecules encoding any of the ACTR polypeptides as described herein may be prepared by a conventional method (e.g., in vitro transcription) and then introduced into suitable host cells, e.g., those described herein, via known methods, e.g., Rabinovich et al., Human Gene Therapy 17:1027-1035.

Following introduction into the host cells a vector encoding any of the ACTR polypeptides provided herein, or the nucleic acid encoding a chimeric vector (e.g., an RNA molecule), the cells may be cultured under conditions that allow for expression of the ACTR polypeptide. In examples in which the nucleic acid encoding the ACTR polypeptide is regulated by a regulatable promoter, the host cells may be cultured in conditions wherein the regulatable promoter is activated. In some embodiments, the promoter is an inducible promoter and the immune cells are cultured in the presence of the inducing molecule or in conditions in which the inducing molecule is produced. Determining whether the ACTR polypeptide is expressed will be evident to one of skill in the art and may be assessed by any known method, for example, detection of the ACTR polypeptide-encoding mRNA by quantitative reverse transcriptase PCR (qRT-PCR) or detection of the ACTR polypeptide protein by methods including Western blotting, fluorescence microscopy, and flow cytometry. Alternatively, expression of the ACTR polypeptide may take place in vivo after the immune cells are administered to a subject.

As used herein, the term “subject” refers to any mammal such as a human, monkey, mouse, rabbit, or domestic mammal. For example, the subject may be a primate. In a preferred embodiment, the subject is human.

Alternatively, expression of an ACTR polypeptide in any of the immune cells disclosed herein can be achieved by introducing RNA molecules encoding the ACTR polypeptides. Such RNA molecules can be prepared by in vitro transcription or by chemical synthesis. The RNA molecules can then introduced into suitable host cells such as immune cells (e.g., T cells, NK cells, or both T cells and NK cells) by, e.g., electroporation. For example, RNA molecules can be synthesized and introduced into host immune cells following the methods described in Rabinovich et al., Human Gene Therapy, 17:1027-1035 and WO WO2013/040557.

In certain embodiments, a vector or RNA molecule comprising the ACTR polypeptide may be introduced to the host cells or immune cells in vivo. As a non-limiting example, this may be accomplished by administering a vector or RNA molecule encoding one or more ACTR polypeptides described herein directly to the subject (e.g., through intravenous administration), producing host cells comprising ACTR polypeptides in vivo.

Methods for preparing host cells expressing any of the ACTR polypeptides described herein may also comprise activating the host cells ex vivo. Activating a host cell means stimulating a host cell into an activated state in which the cell may be able to perform effector functions (e.g., ADCC). Methods of activating a host cell will depend on the type of host cell used for expression of the ACTR polypeptides. For example, T cells may be activated ex vivo in the presence of one or more molecules including, but not limited to: an anti-CD3 antibody, an anti-CD28 antibody, IL-2, and/or phytohemoagglutinin. In other examples, NK cells may be activated ex vivo in the presence of one or molecules such as a 4-1BB ligand, an anti-4-1BB antibody, IL-15, an anti-IL-15 receptor antibody, IL-2, IL12, IL-21, and/or K562 cells. In some embodiments, the host cells expressing any of the ACTR polypeptides (ACTR-expressing cells) described herein are activated ex vivo prior to administration to a subject. Determining whether a host cell is activated will be evident to one of skill in the art and may include assessing expression of one or more cell surface markers associated with cell activation, expression or secretion of cytokines, and cell morphology.

Methods for preparing host cells expressing any of the ACTR polypeptides described herein may comprise expanding the host cells ex vivo. Expanding host cells may involve any method that results in an increase in the number of cells expressing ACTR polypeptides, for example, allowing the host cells to proliferate or stimulating the host cells to proliferate. Methods for stimulating expansion of host cells will depend on the type of host cell used for expression of the ACTR polypeptides and will be evident to one of skill in the art. In some embodiments, the host cells expressing any of the ACTR polypeptides described herein are expanded ex vivo prior to administration to a subject.

In some embodiments, the host cells expressing the ACTR polypeptides are expanded and activated ex vivo prior to administration of the cells to the subject. Host cell activation and expansion may be used to allow integration of a viral vector into the genome and expression of the gene encoding an ACTR polypeptide as described herein. If mRNA electroporation is used, no activation and/or expansion may be required, although electroporation may be more effective when performed on activated cells. In some instances, an ACTR polypeptide is transiently expressed in a suitable host cell (e.g., for 3-5 days). Transient expression may be advantageous if there is a potential toxicity and should be helpful in initial phases of clinical testing for possible side effects.

Any of the host cells expressing the ACTR polypeptides may be mixed with a pharmaceutically acceptable carrier to form a pharmaceutical composition, which is also within the scope of the present disclosure.

The phrase “pharmaceutically acceptable”, as used in connection with compositions of the present disclosure, refers to molecular entities and other ingredients of such compositions that are physiologically tolerable and do not typically produce untoward reactions when administered to a mammal (e.g., a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans. “Acceptable” means that the carrier is compatible with the active ingredient of the composition (e.g., the nucleic acids, vectors, cells, or therapeutic antibodies) and does not negatively affect the subject to which the composition(s) are administered. Any of the pharmaceutical compositions to be used in the present methods can comprise pharmaceutically acceptable carriers, excipients, or stabilizers in the form of lyophilized formations or aqueous solutions.

Pharmaceutically acceptable carriers, including buffers, are well known in the art, and may comprise phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; and/or non-ionic surfactants. See, e.g. Remington: The Science and Practice of Pharmacy 20^{th} Ed. (2000) Lippincott Williams and Wilkins, Ed. K. E. Hoover.

The pharmaceutical compositions of the disclosure may also contain one or more additional active compounds as necessary for the particular indication being treated and/or for the enhancement of ADCC, preferably those with complementary activities that do not adversely affect each other. Non-limiting examples of possible additional active compounds include, e.g., IL-2 as well as various agents known in the field and listed in the discussion of combination treatments, below.

In the context of the present disclosure insofar as it relates to any of the disease conditions recited herein, the terms “treat”, “treatment”, and the like mean to relieve or alleviate at least one symptom associated with such condition, or to slow or reverse the progression of such condition. Within the meaning of the present disclosure, the term “treat” also denotes to arrest, delay the onset (i.e., the period prior to clinical manifestation of a disease) and/or reduce the risk of developing or worsening a disease. For example, in connection with cancer the term “treat” may mean eliminate or reduce a patient's tumor burden, or prevent, delay or inhibit metastasis, etc.

### III. Combined Immunotherapy of Immune Cells Expressing an ACTR Polypeptide and Therapeutic Antibodies

The exemplary ACTR polypeptides of the present disclosure confer antibody-dependent cell cytotoxicity (ADCC) capacity to T lymphocytes and enhance ADCC in NK cells. When the receptor is engaged by an antibody bound to cells, it triggers T-cell activation, sustained proliferation and specific cytotoxicity against the bound cells.

The degree of affinity of CD16 for the Fc portion of Ig is a critical determinant of ADCC and thus to clinical responses to antibody immunotherapy. The CD16 with the V158 polymorphism which has a high binding affinity for Ig and mediates superior ADCC was selected as an example. Although the F158 receptor has lower potency than the V158 receptor in induction of T cell proliferation and ADCC, the F158 receptor may have lower in vivo toxicity than the V158 receptor making it useful in some clinical contexts.

The ACTR polypeptides and methods of the present disclosure facilitate T-cell therapy by allowing one single receptor to be used for all cancers when combined with an antibody that specifically binds a cancer antigen. Antibody-directed cytotoxicity could be stopped whenever required by simple withdrawal of antibody administration. Clinical safety can be further enhanced by using mRNA electroporation to express the ACTR polypeptides transiently, to limit any potential autoimmune reactivity.

Thus, in one embodiment, the disclosure provides a method for enhancing efficacy of an antibody-based immunotherapy of a cancer in a subject in need thereof, which subject is being treated with an antibody which can bind to antigen-expressing cells and has a humanized Fc portion which can bind to human CD16, said method comprising introducing into the subject a therapeutically effective amount an antibody and a therapeutically effective amount of T lymphocytes or NK cells, which T lymphocytes or NK cells comprise an ACTR polypeptide of the disclosure.

As used herein the term “therapeutically effective” applied to dose or amount refers to that quantity of a compound or pharmaceutical composition that is sufficient to result in a desired activity upon administration to a subject in need thereof. Note that when a combination of active ingredients is administered (e.g., a first pharmaceutical composition comprising an antibody, and a second pharmaceutical composition comprising a population of T lymphocytes or NK cells that express an antibody-coupled T-cell receptor (ACTR) construct), the effective amount of the combination may or may not include amounts of each ingredient that would have been effective if administered individually. Within the context of the present disclosure, the term “therapeutically effective” refers to that quantity of a compound or pharmaceutical composition that is sufficient to delay the manifestation, arrest the progression, relieve or alleviate at least one symptom of a disorder treated by the methods of the present disclosure.

### A. Enhancing Immune Therapy Efficacy

Host cells (e.g., immune cells) expressing ACTR polypeptides described herein are useful for enhancing ADCC in a subject and/or for enhancing the efficacy of an antibody-based immunotherapy. In some embodiments, the subject is a mammal, such as a human, monkey, mouse, rabbit, or domestic mammal. In some embodiments, the subject is a human. In some embodiments, the subject is a human cancer patient. In some embodiments, the subject has been treated or is being treated with any of the therapeutic antibodies described herein.

To practice the method described herein, an effective amount of the immune cells (NK cells and/or T lymphocytes) expressing any of the ACTR polypeptides described herein and an effective amount of an antibody, or compositions thereof may be administered to a subject in need of the treatment via a suitable route, such as intravenous administration. As used herein, an effective amount refers to the amount of the respective agent (e.g., the NK cells and/or T lymphocytes expressing ACTR polypeptides, antibodies, or compositions thereof) that upon administration confers a therapeutic effect on the subject. Determination of whether an amount of the cells or compositions described herein achieved the therapeutic effect would be evident to one of skill in the art. Effective amounts vary, as recognized by those skilled in the art, depending on the particular condition being treated, the severity of the condition, the individual patient parameters including age, physical condition, size, gender, sex, and weight, the duration of the treatment, the nature of concurrent therapy (if any), the specific route of administration and like factors within the knowledge and expertise of the health practitioner. In some embodiments, the effective amount alleviates, relieves, ameliorates, improves, reduces the symptoms, or delays the progression of any disease or disorder in the subject. In some embodiments, the subject is a human. In some embodiments, the subject in need of treatment is a human cancer patient.

The methods of the disclosure may be used for treatment of any cancer. Specific non-limiting examples of cancers which can be treated by the methods of the disclosure include, for example, lymphoma, breast cancer, gastric cancer, neuroblastoma, osteosarcoma, lung cancer, skin cancer, prostate cancer, colorectal cancer, renal cell carcinoma, ovarian cancer, rhabdomyosarcoma, leukemia, mesothelioma, pancreatic cancer, head and neck cancer, retinoblastoma, glioma, glioblastoma, thyroid cancer, hepatocellular cancer, esophageal cancer, cervical cancer, and neuroblastoma. In certain embodiments, the cancer may be a solid tumor.

In some embodiments, the immune cells are administered to a subject in an amount effective in enhancing ADCC activity by least 20% and/or by at least 2-fold, e.g., enhancing ADCC by 50%, 80%, 100%, 2-fold, 5-fold, 10-fold, 20-fold, 50-fold, 100-fold, or more.

The immune cells are co-administered with a therapeutic antibody in order to target cells expressing the antigen to which the antibody binds. Antibody-based immunotherapy may be used to treat, alleviate, or reduce the symptoms of any disease or disorder for which the immunotherapy is considered useful in a subject.

An antibody (interchangeably used in plural form) is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody” encompasses not only intact (i.e., full-length) polyclonal or monoclonal antibodies, but also antigen-binding fragments thereof which comprise an Fc region, mutants thereof, fusion proteins comprising an antibody portion, humanized antibodies, chimeric antibodies, diabodies, nanobodies, linear antibodies, multispecific antibodies (e.g., bispecific antibodies) and any other modified configuration of the immunoglobulin molecule that comprises an antigen recognition site of the required specificity and an Fc region, including glycosylation variants of antibodies, amino acid sequence variants of antibodies, and covalently modified antibodies. An antibody includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known. The antibody for use in the present disclosure contains an Fc region recognizable by the co-used ACTR T cells. The Fc region may be a human or humanized Fc region.

Any of the antibodies described herein can be either monoclonal or polyclonal. A “monoclonal antibody” refers to a homogenous antibody population and a “polyclonal antibody” refers to a heterogeneous antibody population. These two terms do not limit the source of an antibody or the manner in which it is made.

In one example, the antibody used in the methods described herein is a humanized antibody. Humanized antibodies refer to forms of non-human (e.g. murine) antibodies that are specific chimeric immunoglobulins, immunoglobulin chains, or antigen-binding fragments thereof that contain minimal sequence derived from non-human immunoglobulin. For the most part, humanized antibodies are human immunoglobulins (recipient antibody) in which residues from a complementary determining region (CDR) of the recipient are replaced by residues from a CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Antibodies may have Fc regions modified as described in WO 99/58572. Other forms of humanized antibodies have one or more CDRs (one, two, three, four, five, six) which are altered with respect to the original antibody, which are also termed one or more CDRs “derived from” one or more CDRs from the original antibody. Humanized antibodies may also involve affinity maturation.

In another example, the antibody described herein is a chimeric antibody, which can include a heavy constant region and a light constant region from a human antibody. Chimeric antibodies refer to antibodies having a variable region or part of variable region from a first species and a constant region from a second species. Typically, in these chimeric antibodies, the variable region of both light and heavy chains mimics the variable regions of antibodies derived from one species of mammals (e.g., a non-human mammal such as mouse, rabbit, and rat), while the constant portions are homologous to the sequences in antibodies derived from another mammal such as a human. In some embodiments, amino acid modifications can be made in the variable region and/or the constant region.

The immune cells (e.g., T lymphocytes and/or NK cells) expressing any of the ACTR polypeptides disclosed herein may be administered to a subject who has been treated or is being treated with an Fc-containing antibody. For example, the immune cells may be administered to a human subject simultaneously with an antibody. Alternatively, the immune cells may be administered to a human subject during the course of an antibody-based immunotherapy. In some examples, the immune cells and an antibody can be administered to a human subject at least 4 hours apart, e.g., at least 12 hours apart, at least 1 day apart, at least 3 days apart, at least one week apart, at least two weeks apart, or at least one month apart.

In some embodiments, the antibodies described herein specifically bind to the corresponding target antigen or an epitope thereof. An antibody that “specifically binds” to an antigen or an epitope is a term well understood in the art. A molecule is said to exhibit “specific binding” if it reacts more frequently, more rapidly, with greater duration and/or with greater affinity with a particular target antigen than it does with alternative targets. An antibody “specifically binds” to a target antigen or epitope if it binds with greater affinity, avidity, more readily, and/or with greater duration than it binds to other substances. For example, an antibody that specifically (or preferentially) binds to an antigen or an antigenic epitope therein is an antibody that binds this target antigen with greater affinity, avidity, more readily, and/or with greater duration than it binds to other antigens or other epitopes in the same antigen. It is also understood with this definition that, for example, an antibody that specifically binds to a first target antigen may or may not specifically or preferentially bind to a second target antigen. As such, “specific binding” or “preferential binding” does not necessarily require (although it can include) exclusive binding. In some examples, an antibody that “specifically binds” to a target antigen or an epitope thereof may not bind to other antigens or other epitopes in the same antigen. In some embodiments, the antibodies described herein specifically bind to TNF-alpha, HER2, CD52, CD38, BCMA, GPC3, PDGF-R-alpha, CD25, VEGF, BLyS, CD30, ILl-B, EGFR, RANK ligand, GD2, C5, CD11a, CD22, CD33, CTLA4, CEACAM5, alpha-4 integrin, CD20, CD19, IgE, RSV, VEGFR2, IL6R, IL12, IL23, integrin alpha4-beta7, or PSMA.

In some embodiments, an antibody as described herein has a suitable binding affinity for the target antigen (e.g., TNF-alpha, HER2, CD52, CD38, BCMA, GPC3, PDGF-R-alpha, CD25, VEGF, BLyS, CD30, ILl-B, EGFR, RANK ligand, GD2, C5, CD11a, CD22, CD33, CTLA4, CEACAM5, alpha-4 integrin, CD20, CD19, IgE, RSV, VEGFR2, IL6R, IL12, IL23, integrin alpha4-beta7, or PSMA) or antigenic epitopes thereof. As used herein, “binding affinity” refers to the apparent association constant or K_{A}. The K_{A} is the reciprocal of the dissociation constant (K_{D}). The antibody for use in the methods described herein may have a binding affinity (K_{D}) of at least 10⁻⁵, 10⁻⁶, 10⁻⁷, 10⁻, 10⁻⁹, 10⁻¹⁰ M, or lower for the target antigen or antigenic epitope. An increased binding affinity corresponds to a decreased K_{D}. Higher affinity binding of an antibody for a first antigen relative to a second antigen can be indicated by a higher K_{A} (or a smaller numerical value K_{D}) for binding the first antigen than the K_{A} (or numerical value K_{D}) for binding the second antigen. In such cases, the antibody has specificity for the first antigen (e.g., a first protein in a first conformation or mimic thereof) relative to the second antigen (e.g., the same first protein in a second conformation or mimic thereof; or a second protein). Differences in binding affinity (e.g., for specificity or other comparisons) can be at least 1.5, 2, 3, 4, 5, 10, 15, 20, 37.5, 50, 70, 80, 91, 100, 500, 1000, 10,000 or 10⁵ fold. In some embodiments, any of the antibodies may be further affinity matured to increase the binding affinity of the antibody to the target antigen or antigenic epitope thereof.

Binding affinity (or binding specificity) can be determined by a variety of methods including equilibrium dialysis, equilibrium binding, gel filtration, ELISA, surface plasmon resonance, or spectroscopy (e.g., using a fluorescence assay). Exemplary conditions for evaluating binding affinity are in HBS-P buffer (10 mM HEPES pH7.4, 150 mM NaCl, 0.005% (v/v) Surfactant P20). These techniques can be used to measure the concentration of bound binding protein as a function of target protein concentration. The concentration of bound binding protein ([Bound]) is generally related to the concentration of free target protein ([Free]) by the following equation:

[Bound]=[Free]/(Kd+[Free])

It is not always necessary to make an exact determination of K_{A}, though, since sometimes it is sufficient to obtain a quantitative measurement of affinity, e.g., determined using a method such as ELISA or FACS analysis, is proportional to K_{A}, and thus can be used for comparisons, such as determining whether a higher affinity is, e.g., 2-fold higher, to obtain a qualitative measurement of affinity, or to obtain an inference of affinity, e.g., by activity in a functional assay, e.g., an in vitro or in vivo assay.

The antibodies for use in the immune therapy methods described herein may bind to (e.g., specifically bind to) a specific region or an antigenic epitope therein.

Exemplary antibodies for use with the compositions and methods described herein include antibodies specific to TNF-alpha, HER2, CD52, CD38, BCMA, GPC3, PDGF-R-alpha, CD25, VEGF, BLyS, CD30, ILl-B, EGFR, RANK ligand, GD2, C5, CD11a, CD22, CD33, CTLA4, CEACAM5, alpha-4 integrin, CD20, CD19, IgE, RSV, VEGFR2, IL6R, IL12, IL23, integrin alpha4-beta7, or PSMA, as well as other known antibodies. As a non-limiting example, antibodies for use with the compositions and methods described herein may be one or more of the following: Adalimumab, Ado-Trastuzumab emtansine, Alemtuzumab, Atezolizumab, Avelumab, Basiliximab, Bevacizumab, Belimumab, Brentuximab vedotin, Canakinumab, Cetuximab, Daclizumab, Daratumumab, Denosumab, Dinutuximab, Durvalumab, Eculizumab, Efalizumab, Epratuzumab, Gemtuzumab, Golimumab, Infliximab, Ipilimumab, Labetuzumab, Natalizumab, Obinutuzumab, Ofatumumab, Olaratumab, Omalizumab, Palivizumab, Panitumumab, Pertuzumab, Ramucirumab, Rituximab, Tocilizumab, Trastuzumab, Ustekinumab, and Vedolizumab.

The efficacy of an antibody-based immunotherapy may be assessed by any method known in the art and would be evident to a skilled medical professional. For example, the efficacy of the antibody-based immunotherapy may be assessed by survival of the subject or tumor or cancer burden in the subject or tissue or sample thereof. In some embodiments, the immune cells are administered to a subject in need of the treatment in an amount effective in enhancing the efficacy of an antibody-based immunotherapy by at least 20% and/or by at least 2-fold, e.g., enhancing the efficacy of an antibody-based immunotherapy by 50%, 80%, 100%, 2-fold, 5-fold, 10-fold, 20-fold, 50-fold, 100-fold or more, as compared to the efficacy in the absence of the immune cells expressing the ACTR polypeptide and/or the antibody.

In any of the compositions or methods described herein, the immune cells (e.g., NK and/or T cells) may be autologous to the subject, i.e., the immune cells may be obtained from the subject in need of the treatment, genetically engineered for expression of the ACTR polypeptides, and then administered to the same subject. In one specific embodiment, prior to re-introduction into the subject, the autologous immune cells (e.g., T lymphocytes or NK cells) are activated and/or expanded ex vivo. Administration of autologous cells to a subject may result in reduced rejection of the host cells as compared to administration of non-autologous cells.

Alternatively, the host cells are allogeneic cells, i.e., the cells are obtained from a first subject, genetically engineered for expression of the ACTR polypeptide, and administered to a second subject that is different from the first subject but of the same species. For example, allogeneic immune cells may be derived from a human donor and administered to a human recipient who is different from the donor. In a specific embodiment, the T lymphocytes are allogeneic T lymphocytes in which the expression of the endogenous T cell receptor has been inhibited or eliminated. In one specific embodiment, prior to introduction into the subject, the allogeneic T lymphocytes are activated and/or expanded ex vivo. T lymphocytes can be activated by any method known in the art, e.g., in the presence of anti-CD3/CD28, IL-2, and/or phytohemoagglutinin.

NK cells can be activated by any method known in the art, e.g., in the presence of one or more agents selected from the group consisting of CD137 ligand protein, CD137 antibody, IL-15 protein, IL-15 receptor antibody, IL-2 protein, IL-12 protein, IL-21 protein, and K562 cell line. See, e.g., U.S. Pat. Nos. 7,435,596 and 8,026,097 for the description of useful methods for expanding NK cells. For example, NK cells used in the compositions or methods of the disclosure may be preferentially expanded by exposure to cells that lack or poorly express major histocompatibility complex I and/or II molecules and which have been genetically modified to express membrane bound IL-15 and 4-1BB ligand (CDI37L). Such cell lines include, but are not necessarily limited to, K562 [ATCC, CCL 243; Lozzio et al., Blood 45(3): 321-334 (1975); Klein et al., Int. J. Cancer 18: 421-431 (1976)], and the Wilms tumor cell line HFWT (Fehniger et al., Int Rev Immunol 20(3-4):503-534 (2001); Harada H, et al., Exp Hematol 32(7):614-621 (2004)), the uterine endometrium tumor cell line HHUA, the melanoma cell line HMV-II, the hepatoblastoma cell line HuH-6, the lung small cell carcinoma cell lines Lu-130 and Lu-134-A, the neuroblastoma cell lines NB 19 and N1369, the embryonal carcinoma cell line from testis NEC 14, the cervix carcinoma cell line TCO-2, and the bone marrow-metastasized neuroblastoma cell line TNB 1 [Harada, et al., Jpn. J. Cancer Res 93: 313-319 (2002)]. Preferably the cell line used lacks or poorly expresses both MHC I and II molecules, such as the K562 and HFWT cell lines. A solid support may be used instead of a cell line. Such support should preferably have attached on its surface at least one molecule capable of binding to NK cells and inducing a primary activation event and/or a proliferative response or capable of binding a molecule having such an affect thereby acting as a scaffold. The support may have attached to its surface the CD137 ligand protein, a CD137 antibody, the IL-15 protein or an IL-15 receptor antibody. Preferably, the support will have IL-15 receptor antibody and CD137 antibody bound on its surface.

In one embodiment of the described compositions or methods, introduction (or re-introduction) of T lymphocytes, NK cells, or T lymphocytes and NK cells to the subject is followed by administering to the subject a therapeutically effective amount of IL-2.

In accordance with the present disclosure, patients can be treated by infusing therapeutically effective doses of immune cells such as T lymphocytes or NK cells comprising an ACTR polypeptide of the disclosure in the range of about 10⁵ to 10¹⁰ or more cells per kilogram of body weight (cells/Kg). The infusion can be repeated as often and as many times as the patient can tolerate until the desired response is achieved. The appropriate infusion dose and schedule will vary from patient to patient, but can be determined by the treating physician for a particular patient. Typically, initial doses of approximately 10⁶ cells/Kg will be infused, escalating to 10⁸ or more cells/Kg. IL-2 can be co-administered to expand infused cells. The amount of IL-2 can about 1-5×10⁶ international units per square meter of body surface.

In some embodiments, the antibody is administered to the subject in one or more doses of about 100-500 mg, 500-1000 mg, 1000-1500 mg or 1500-2000 mg. In some embodiments, the antibody is administered to the subject in one or more doses of about 500 mg, about 600 mg, about 700 mg, about 800 mg, or about 900 mg. In some embodiments, the antibody is administered to the subject in one or more doses of about 1000 mg, about 1100 mg, about 1200 mg, about 1300 mg, about 1400 mg, about 1500 mg, about 1600 mg, about 1700 mg, or about 1800 mg. In some embodiments, the antibody is administered to the subject in one or more doses of about 1600 mg.

The particular dosage regimen, i.e., dose, timing and repetition, used in the method described herein will depend on the particular subject and that subject's medical history. The appropriate dosage of the antibody used will depend on the type of cancer to be treated, the severity and course of the disease, previous therapy, the patient's clinical history and response to the antibody, and the discretion of the attending physician. The antibody can be administered to the patient at one time or over a series of treatments. The progress of the therapy of the disclosure can be easily monitored by conventional techniques and assays.

The administration of the antibody can be performed by any suitable route, including systemic administration as well as administration directly to the site of the disease (e.g., to a tumor).

In some embodiments, the method involves administering the antibody to the subject in one dose. In some embodiments, the method involves administering the antibody to the subject in multiple dose (e.g., at least 2, 3, 4, 5, 6, 7, or 8 doses). In some embodiments, the antibody is administered to the subject in multiple doses, with the first dose of the antibody administered to the subject about 1, 2, 3, 4, 5, 6, or 7 days prior to administration of the immune cells expressing ACTR. In some embodiments, the first dose of the antibody is administered to the subject between about 24-48 hours prior to the administration of the immune cells expressing ACTR.

In some embodiments, the antibody is administered to the subject prior to administration of the immune cells expressing the ACTR and then subsequently about every two weeks. In some embodiments, the first two doses of the antibody are administered about one week (e.g., about 6, 7, 8, or 9 days) apart. In certain embodiments, the third and following doses are administered about every two weeks.

In any of the embodiments described herein, the timing of the administration of the antibody is approximate and includes three days prior to and three days following the indicated day (e.g., administration every three weeks encompasses administration on day 18, day 19, day 20, day 21, day 22, day 23, or day 24).

The efficacy of the compositions or methods described herein may be assessed by any method known in the art and would be evident to a skilled medical professional. For example, the efficacy of the antibody-based immunotherapy may be assessed by survival of the subject or cancer burden in the subject or tissue or sample thereof. In some embodiments, the antibody based immunotherapy is assessed based on the safety or toxicity of the therapy (e.g., administration of the antibody and the immune cells expressing ACTR polypeptides) in the subject, for example by the overall health of the subject and/or the presence of adverse events or severe adverse events.

### B. Combination Treatments

The compositions and methods described in the present disclosure may be utilized in conjunction with other types of therapy for cancer, such as chemotherapy, surgery, radiation, gene therapy, and so forth. Such therapies can be administered simultaneously or sequentially (in any order) with the immunotherapy according to the present disclosure.

When co-administered with an additional therapeutic agent, suitable therapeutically effective dosages for each agent may be lowered due to the additive action or synergy.

The treatments of the disclosure can be combined with other immunomodulatory treatments such as, e.g., therapeutic vaccines (including but not limited to GVAX, DC-based vaccines, etc.), checkpoint inhibitors (including but not limited to agents that block CTLA4, PD1, LAG3, TIM3, etc.) or activators (including but not limited to agents that enhance 41BB, OX40, etc.).

Non-limiting examples of other therapeutic agents useful for combination with the immunotherapy of the disclosure include: (i) anti-angiogenic agents (e.g., TNP-470, platelet factor 4, thrombospondin-1, tissue inhibitors of metalloproteases (TIMP1 and TIMP2), prolactin (16-Kd fragment), angiostatin (38-Kd fragment of plasminogen), endostatin, bFGF soluble receptor, transforming growth factor beta, interferon alpha, soluble KDR and FLT-1 receptors, placental proliferin-related protein, as well as those listed by Carmeliet and Jain (2000)); (ii) a VEGF antagonist or a VEGF receptor antagonist such as anti-VEGF antibodies, VEGF variants, soluble VEGF receptor fragments, aptamers capable of blocking VEGF or VEGFR, neutralizing anti-VEGFR antibodies, inhibitors of VEGFR tyrosine kinases and any combinations thereof; and (iii) chemotherapeutic compounds such as, e.g., pyrimidine analogs (5-fluorouracil, floxuridine, capecitabine, gemcitabine and cytarabine), purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristine, vinblastine, nocodazole, epothilones, and navelbine, epidipodophyllotoxins (etoposide and teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethylmelamine oxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycin, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (brefeldin); immunosuppressives (cyclosporine, tacrolimus (FK-506), sirolimus (rapamycin), azathioprine, mycophenolate mofetil); anti-angiogenic compounds (e.g., TNP-470, genistein, bevacizumab) and growth factor inhibitors (e.g., fibroblast growth factor (FGF) inhibitors); angiotensin receptor blocker; nitric oxide donors; anti-sense oligonucleotides; antibodies (trastuzumab); cell cycle inhibitors and differentiation inducers (tretinoin); AKT inhibitors (such as MK-2206 2HCl, Perifosine (KRX-0401), GSK690693, Ipatasertib (GDC-0068), AZD5363, uprosertib, afuresertib, or triciribine); mTOR inhibitors, topoisomerase inhibitors (doxorubicin (adriamycin), amsacrine, camptothecin, daunorubicin, dactinomycin, eniposide, epirubicin, etoposide, idarubicin, mitoxantrone, topotecan, and irinotecan), corticosteroids (cortisone, dexamethasone, hydrocortisone, methylprednisolone, prednisone, and prednisolone); growth factor signal transduction kinase inhibitors; mitochondrial dysfunction inducers and caspase activators; and chromatin disruptors.

For examples of additional useful agents see also Physician's Desk Reference, 59.sup.th edition, (2005), Thomson P D R, Montvale N.J.; Gennaro et al., Eds. Remington's The Science and Practice of Pharmacy 20th edition, (2000), Lippincott Williams and Wilkins, Baltimore Md.; Braunwald et al., Eds. Harrison's Principles of Internal Medicine, 15.sup.th edition, (2001), McGraw Hill, NY; Berkow et al., Eds. The Merck Manual of Diagnosis and Therapy, (1992), Merck Research Laboratories, Rahway N.J.

C. Treatment Regimen of Solid Tumors Using ACTR-T Cells in Combination with an Anti-Tumor Antibody

The methods described herein are also based at least in part on the finding that the combination of immune cells expressing ACTRs (e.g., the ACTR of SEQ ID NO:9) and an anti-tumor antibody (e.g., rituximab or trastuzumab), results in proliferation and activation of the immune cells in response to the antibodies binding target cancer cells expressing a tumor antigen such as CD20 or HER2, and that the proliferation and activation are antibody-dependent and self-limiting. The dependence on adequate exposure to the anti-tumor antibody (e.g., rituximab or trastuzumab) indicates that the activity of the immune cells expressing the ACTRs can be modulated by the antibody dose and dosing schedule, providing an advantage of the methods described herein over the previously used CAR T cells.

Accordingly, the present disclosure also provides treatment regimens using the combination of ACTR-T cells and an anti-tumor (e.g., anti-CD20 or anti-HER2) antibody for treating solid tumors (e.g., lymphoma, in particular, relapsed and/or refractory lymphoma, or HER2⁺ cancers such as HER2⁺ breast cancer, gastric cancer, and esophageal cancer). In this treatment, a subject in need of the treatment may be subject to a conditioning regimen (e.g., lymphodepleting therapy) following by the combined anti-tumor antibody (e.g., an anti-CD20 antibody or anti-HER2 antibody)/ACTR-T cell therapy, which comprises administration of an anti-tumor antibody (e.g., an anti-CD20 antibody such as rituximab or an anti-HER2 antibody such as trastuzumab) and infusion of immune cells (e.g., T cells) expressing an ACTR. Below is an illustrative and non-limiting example using T cells expressing an ACTR polypeptide having a CD28 co-stimulatory domain (e.g., SEQ ID NO:9) and an anti-CD20 antibody (e.g., rituximab).

A subject suitable for the treatment may be identified by routine medical practice. Such a subject may be a human patient having CD20+ lymphoma, in particular, relapsed or refractory CD20⁺ lymphoma. A lymphoma refers to a group of blood cell tumors that develop from lymphatic cells. Hodgkin lymphoma and non-Hodgkin lymphoma are the two major types of lymphomas. A lymphoma may be considered “refractory” if lymphoma cells are present in the bone marrow of the subject after having undergone a treatment for the lymphoma. Alternatively, a lymphoma is considered “relapsed” if a return of lymphoma cells is detected in the bone marrow and there is a decrease in the number of normal blood cells after remission of the lymphoma. In some embodiments, the relapsed or refractory CD20+ lymphoma is a non-Hodgkin's lymphoma. In some embodiments, the CD20+ B-cell lymphoma is diffuse large B-cell lymphoma (DLBCL), mantle cell lymphoma (MCL), primary mediastinal B cell lymphoma (PMBCL), grade 3b follicular lymphoma (Gr3b-FL), or transformed histology follicular lymphoma (TH-FL).

Prior to the Anti-CD20/ACTR treatment, a subject such as a human solid tumor (e.g., lymphoma) patient may subject to a conditioning regimen, such as a lymphodepleting therapy to reduce or deplete the endogenous lymphocyte of the subject.

Lymphodepletion refers to the destruction of endogenous lymphocytes and/or T cells, which is commonly used prior to immunotransplantation and immunotherapy. Lymphodepletion can be achieved by irradiation and/or chemotherapy. A “lymphodepleting agent” can be any molecule capable of reducing, depleting, or eliminating endogenous lymphocytes and/or T cells when administered to a subject. In some embodiments, the lymphodepleting agents are administered in an amount effective in reducing the number of lymphocytes by at least 10%, 20%, 30%, 40%, 50%, 60%, 70%, 80%, 90%, 95%, 96%, 96%, 97%, 98%, or at least 99% as compared to the number of lymphocytes prior to administration of the agents. In some embodiments, the lymphodepleting agents are administered in an amount effective in reducing the number of lymphocytes such that the number of lymphocytes in the subject is below the limits of detection. In some embodiments, the subject is administered at least one (e.g., 2, 3, 4, 5 or more) lymphodepleting agents. In some embodiments, the lymphodepleting agents are cytotoxic agents that specifically kill lymphocytes. Examples of lymphodepleting agents include, without limitation, fludarabine, cyclophosphamide, bendamustin, 5-fluorouracil, gemcitabine, methotrexate, dacarbazine, melphalan, doxorubicin, vinblastine, cisplatin, oxaliplatin, paclitaxel, docetaxel, irinotecan, etopside phosphate, mitoxantrone, cladribine, denileukin diftitox, or DAB-IL-2. In some instances, the lymphodepleting agent may be accompanied with low-dose irradiation. The lymphodepletion effect of the conditioning regimen can be monitored via routine practice.

In some embodiments, the lymphodepleting therapy comprises one or more lymphodepleting agents, for example, fludarabine and cyclophosphamide. A subject to be treated by the method described herein may receive multiple doses of the one or more lymphodepleting agents for a suitable period (e.g., 2-5 days) in the conditioning stage.

Following the conditioning regimen (lymphodepleting therapy), the subject is subject to an anti-CD20/ACTR treatment regimen, which comprises administration of an anti-CD20 antibody such as rituximab and infusion of immune cells (e.g., T cells) expressing an ACTR.

An anti-CD20 treatment can be performed on the subject as described herein prior to the treatment of ACTR-expressing immune cells. CD-20 is a B lymphocyte antigen expressed on the surface of B cells of all stages. CD20 positive cells are found in cases of Hodgkins disease, myeloma, and thymoma. Human CD20 is encoded by the MS4A1 gene. Any anti-CD20 antibody known in the art may be used in the methods provided herein. An anti-CD20 antibody is an immunoglobulin molecule capable of specific binding to a CD20 molecule, for example, a CD20 molecule expressed on the surface of B cells. In one example, the anti-CD20 antibody is rituximab. Conventional methods, known to those of ordinary skill in the art of medicine, can be used to administer the anti-CD20 antibody-containing pharmaceutical composition to the subject in need of the treatment. This composition can also be administered via other conventional routes, e.g., administered parenterally. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques.

Following the anti-CD20 treatment, the subject receives ACTR-expressing immune cells such as T cells via, e.g., infusion. The T cells expressing the ACTR may be administered to the subject at any therapeutically effective dose. As a set of non-limiting examples, the T cells expressing the ACTR may be administered to the subject at a dose of 40×10⁶ cells, 80×10⁶ cells, 150×10⁶ cells, or 300×10⁶ cells. One or more than one dose of T cells expressing the ACTR (e.g., 1, 2, 3, 4, 5, 6, or 7 doses) may be administered to the same subject.

Any of the ACTR constructs described herein may be used in this method. In some embodiments, the ACTR construct used in this treat may comprise an extracellular ligand binding domain of an Fc receptor such as CD16 (e.g., the CD16V isoform), a hinge domain of CD28, a transmembrane domain of CD28, a co-stimulatory domain of CD28, and a cytoplasmic signaling domain of CD3ζ. In in particular example, the ACTR construct can be SEQ ID NO:9.

Following the ACTR-T cell treatment, one or more cycles of anti-CD20 treatment may be performed if necessary, which can be determined by a physician. For example, one additional cycle of anti-CD20 antibody treatment can be performed after the ACTR-T cell treatment. Dose-Limiting Toxicity (DLT) assessment can be performed on the subject. The subject can then be subject to another cycle of anti-CD20 antibody treatment followed by response assessment. The anti-CD20 antibody treatment may continue for an additional 21 days until disease progression is observed.

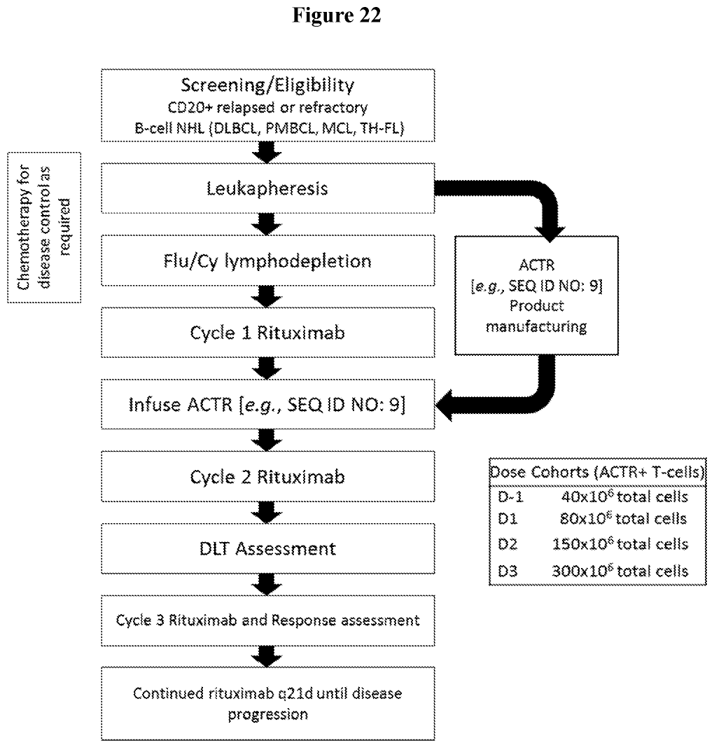

FIG. 22 is a graphic depicting an exemplary treatment schedule for treating patients with relapsed or refractory CD20+ B cell lymphoma using ACTR T cells in combination with rituximab. Subjects with histologically-confirmed relapsed or refractory CD20+ B cell lymphoma of one of the following histologic subtypes could be eligible: DLBCL, MCL, PMBCL, Gr3b-FL, TH-FL.

The efficacy of the methods described herein may be assessed by any method known in the art and would be evident to a skilled medical professional. For example, the efficacy of the antibody-based immunotherapy may be assessed by survival of the subject or cancer burden in the subject or tissue or sample thereof. In some embodiments, the antibody based immunotherapy is assessed based on the safety or toxicity of the therapy (e.g., administration of the anti-CD20 antibody and the immune cells expressing the ACTRs) in the subject, for example by the overall health of the subject and/or the presence of adverse events or severe adverse events.

### D. Treating Diseases Involving Antigens of Activated T Cells

Traditional CAR-T therapy involves the use of CAR constructs specific to cell surface antigens, thereby eliminating pathological cells expressing such antigens. When CAR-T cells target surface antigens that also present on activated T cells, for example, CD5, CD38, or CD7, the in vitro expansion of such CAR-T cells would be impaired because activated CAR-T cells also express such surface antigens and would be killed by other CAR-T cells (fratricide effects). Thus, development of CAR-T therapy is limited by the cell antigen to which it targets.

The ACTR-T therapy has no such antigen limitation. ACTR-T cells do not target pathological cells directly and use antibodies as intermediates. Accordingly, in vitro expansion of ACTR-T cells is not limited to the type of antigen to which the ACTR-T cells target.

Accordingly, the instant disclosure also provides a method for inducing cytotoxicity in a subject, involving the combined use of an antibody specific to an antigen expressed on the surface of activated T cells; and T cells expressing an antibody-coupled T cell receptor (ACTR). This method would benefit treatment of diseases involving cells that express surface antigens, which are also present on activated T cells.

Any ACTR constructs, e.g., those known in the art or disclosed herein, may be used in this method. For example, the ACTR constructs for use in this method may comprise: (a) an Fc binding domain; (b) a transmembrane domain; (c) at least one co-stimulatory signaling domain; and (d) a cytoplasmic signaling domain comprising an immunoreceptor tyrosine-based activation motif (ITAM), wherein either (c) or (d) is located at the C-terminus of the chimeric receptor. In some embodiments, the ACTR may further comprise a hinge domain, which can be located between (a) and (b).

Any of the Fc-binding domains, transmembrane domains, cytoplasmic signaling domains, and/or hinge domains described herein can be used for constructing the ACTR for use in this method.

In addition to the co-stimulatory domain of CD28 described above, other co-stimulatory domains also can be used for making the ACTR constructs for use in this method. Examples include, without limitation, members of the B7/CD28 family (e.g., B7-1/CD80, B7-2/CD86, B7-H1/PD-L1, B7-H2, B7-H3, B7-H4, B7-H6, B7-H7, BTLA/CD272, CD28, CTLA-4, Gi24/VISTA/B7-H5, ICOS/CD278, PD-1, PD-L2/B7-DC, and PDCD6); members of the TNF superfamily (e.g., 4-1BB/TNFSF9/CD137, 4-1BB Ligand/TNFSF9, BAFF/BLyS/TNFSF13B, BAFF R/TNFRSF13C, CD27/TNFRSF7, CD27 Ligand/TNFSF7, CD30/TNFRSF8, CD30 Ligand/TNFSF8, CD40/TNFRSF5, CD40/TNFSF5, CD40 Ligand/TNFSF5, DR3/TNFRSF25, GITR/TNFRSF18, GITR Ligand/TNFSF18, HVEM/TNFRSF14, LIGHT/TNFSF14, Lymphotoxin-alpha/TNF-beta, OX40/TNFRSF4, OX40 Ligand/TNFSF4, RELT/TNFRSF19L, TACI/TNFRSF13B, TL1A/TNFSF15, TNF-alpha, and TNF RII/TNFRSF1B); members of the SLAM family (e.g., 2B4/CD244/SLAMF4, BLAME/SLAMF8, CD2, CD2F-10/SLAMF9, CD48/SLAMF2, CD58/LFA-3, CD84/SLAMF5, CD229/SLAMF3, CRACC/SLAMF7, NTB-A/SLAMF6, and SLAM/CD150); and any other co-stimulatory molecules, such as CD2, CD7, CD53, CD82/Kai-1, CD90/Thy1, CD96, CD160, CD200, CD300a/LMIR1, HLA Class I, HLA-DR, Ikaros, Integrin alpha 4/CD49d, Integrin alpha 4 beta 1, Integrin alpha 4 beta 7/LPAM-1, LAG-3, TCL1A, TCL1B, CRTAM, DAP12, Dectin-1/CLEC7A, DPPIV/CD26, EphB6, TIM-1/KIM-1/HAVCR, TIM-4, TSLP, TSLP R, lymphocyte function associated antigen-1 (LFA-1), and NKG2C. In some embodiments, the co-stimulatory signaling domain is of 4-1BB, CD28, OX40, ICOS, CD27, GITR, HVEM, TIM 1, LFA1(CD11a) or CD2, or any variant thereof.

Exemplary ACTR constructs for use with the methods for inducing cytotoxicity in a subject may be found, for example, in the instant description and figures (e.g., SEQ ID NO: 9) or may be found in International Patent Application No.: PCT/US2015/049126, which is incorporated by reference herein for this purpose.

In some embodiments, the ACTR-T cells used in the method for inducing cytotoxicity are expanded in vitro, for example, following the methods described herein.

Antibodies specific to antigens expressed on the surface of T cells may include antibodies to any antigen expressed on the surface of T cells. For example, the antibody may bind to CD38 or CD7. As another set of non-limiting examples, the antibody may bind to CD2, CD3, or CD5. Antibodies binding an antigen expressed on the surface of a T cell may include, but are not limited to, daratumumab, SAR650984, siplizumab, BTI-322, otelixizumab, teplizumab, visilizumab, zolimomab aritox, telimomab aritox.

### IV. Kits for Therapeutic Use

The present disclosure also provides kits for use of the compositions described herein. For example, the present disclosure also provides kits for use of an antibody and a population of immune cells (e.g., T lymphocytes or NK cells) that express an antibody-coupled T-cell receptor (ACTR) construct in enhancing antibody-dependent cell-mediated cytotoxicity and enhancing an antibody-based immunotherapy. Such kits may include one or more containers comprising a first pharmaceutical composition that comprises an antibody and a pharmaceutically acceptable carrier, and a second pharmaceutical composition that comprises a population of T lymphocytes and/or NK cells that express an antibody-coupled T-cell receptor (ACTR) construct such as those described herein. The population of T lymphocytes and/or NK cells may further express an exogenous polypeptide comprising a co-stimulatory domain or a ligand of a co-stimulatory factor.

In some embodiments, the kit described herein comprises ACTR-T cells which are expanded in vitro, and an antibody specific to a cell surface antibody that is present on activated T cells, for example, an anti-CD5 antibody, an anti-CD38 antibody or an anti-CD7 antibody. The ACTR-T cells may express any of the ACTR constructs known in the art or disclosed herein. In one example, the ACTR-T cells express ACTR variant SEQ ID NO: 9.

In some embodiments, the kit can additionally comprise instructions for use in any of the methods described herein. The included instructions may comprise a description of administration of the first and second pharmaceutical compositions to a subject to achieve the intended activity, e.g., enhancing ADCC activity, and/or enhancing the efficacy of an antibody-based immunotherapy, in a subject. The kit may further comprise a description of selecting a subject suitable for treatment based on identifying whether the subject is in need of the treatment. In some embodiments, the instructions comprise a description of administering the first and second pharmaceutical compositions to a subject who is in need of the treatment.

The instructions relating to the use of the first and second pharmaceutical compositions described herein generally include information as to dosage, dosing schedule, and route of administration for the intended treatment. The containers may be unit doses, bulk packages (e.g., multi-dose packages) or sub-unit doses. Instructions supplied in the kits of the disclosure are typically written instructions on a label or package insert. The label or package insert indicates that the pharmaceutical compositions are used for treating, delaying the onset, and/or alleviating a disease or disorder in a subject.

The kits provided herein are in suitable packaging. Suitable packaging includes, but is not limited to, vials, bottles, jars, flexible packaging, and the like. Also contemplated are packages for use in combination with a specific device, such as an inhaler, nasal administration device, or an infusion device. A kit may have a sterile access port (for example, the container may be an intravenous solution bag or a vial having a stopper pierceable by a hypodermic injection needle). The container may also have a sterile access port. At least one active agent in the first pharmaceutical composition is an antibody as described herein. At least one active agent in the second pharmaceutical composition is a population of immune cells (e.g., T lymphocytes or NK cells) that express an antibody-coupled T-cell receptor (ACTR) construct as described herein.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (1RL Press, (1986»; and B. Perbal, A practical Guide To Molecular Cloning (1984); F. M. Ausubel et al. (eds.).

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1: Cytotoxicity Assay of ACTR-T Cells Against Target Cells

Gamma-retroviruses were generated that encoded the ACTR variants in Table 3. These viruses were used to infect primary human T-cells, generating cells that expressed these ACTR variants on the surface of infected cells. The cells were subsequently used in cytotoxicity assays with CD20-positive Raji target cells that constitutively expressed firefly luciferase and CD20-targeting rituximab or in HER2-positive HCC1954 cells that constitutively expressed firefly luciferase and HER2-targeting trastuzumab.

T-cells (effector; E) and Raji target cells (target; T) were incubated at a 4:1 effector-to-target ratio (120,000 effector cells; 30,000 target cells) in the presence of different concentrations of rituximab (0-10 μg/mL) in a 200-μL reaction volume in RPMI 1640 media supplemented with 10% fetal bovine serum. All reactions were carried out in duplicate. Reactions were incubated in a CO₂ (5%) incubator at 37 degrees C. for 40-48 hours. A 100-μL volume of supernatant was removed from each reaction. A 100-μL volume of Bright-Glo luciferase assay reagent (Promega; Madison, Wis.) was added to the remaining reaction and incubated at room temperature for 10 minutes. Luminescence was then measured using an Envision multilabel reader (PerkinElmer; Waltham, Mass.). The percentage of live cells was determined by dividing the luminescence signal of a given sample by the luminescence signal in the absence of antibody for each T-cell type and multiplying by 100. The percent cytotoxicity was determined by subtracting the percent live cells from 100.

In a separate experiment, T-cells (effector; E) and HCC1954 target cells (target; T) were incubated at a 1:4 effector-to-target ratio (30,000 effector cells; 30,000 target cells) in the presence of different concentrations of trastuzumab (0-1 μg/mL) in a 200-μL reaction volume in RPMI 1640 media supplemented with 10% fetal bovine serum. All reactions were carried out in duplicate. Reactions were incubated in a CO₂ (5%) incubator at 37 degrees C. for 20-24 hours. Luciferase assays were performed, luminescence was measured, and percent cytotoxicity were determined as above.

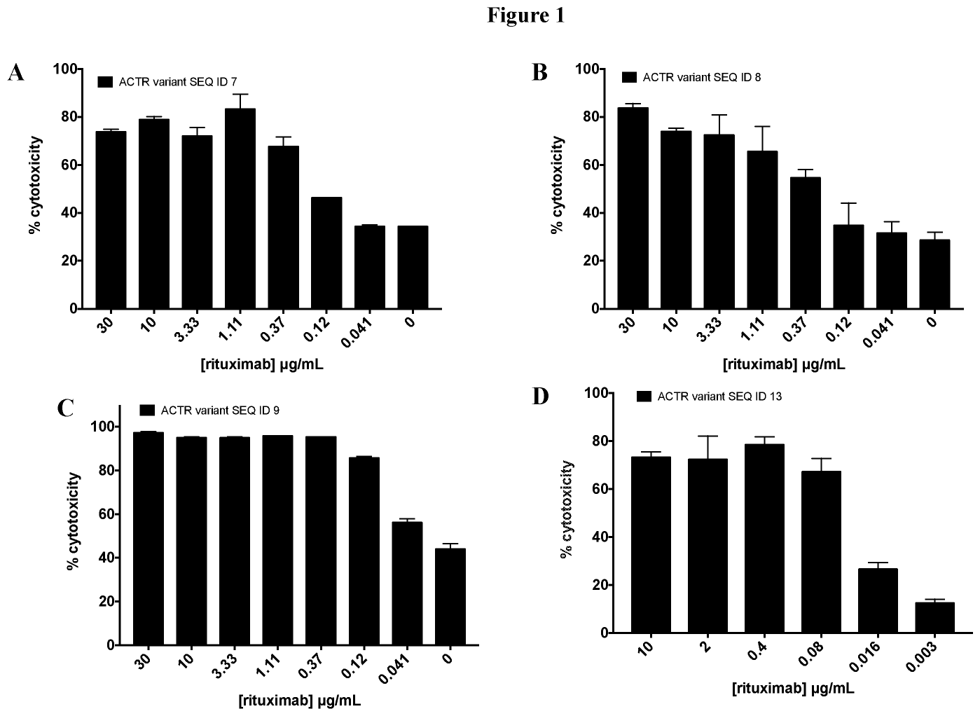

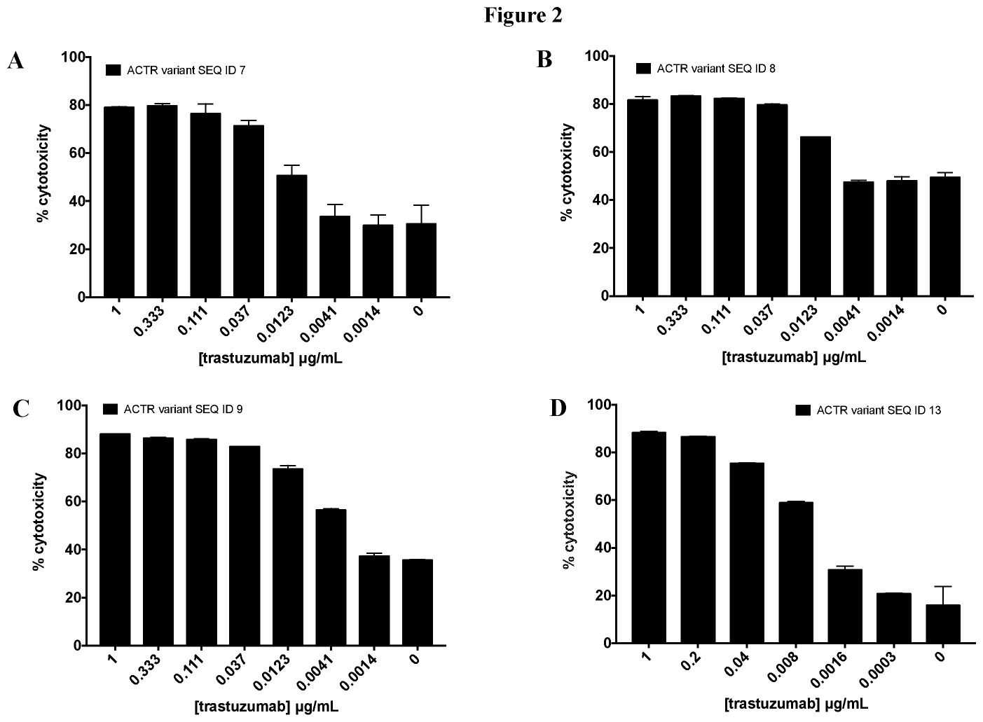

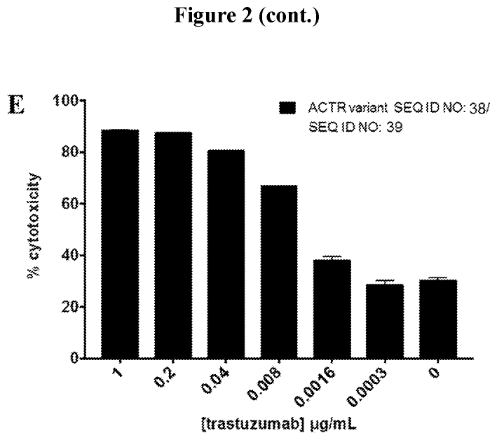

When target Raji cells were incubated with ACTR T-cells expressing variants of SEQ ID Nos: 7, 8, 9, and 13, and increasing concentrations of rituximab, a concentration-dependent increase in cytotoxicity was observed (FIG. 1). When target HCC1954 cells were incubated with ACTR T-cells expressing nucleic acids encoding variants of SEQ ID NOs: 7, 8, 9, 13, and SEQ ID NO: 38/SEQ ID NO: 39 and increasing concentrations of trastuzumab, a concentration-dependent increase in cytotoxicity was also observed (FIG. 2).

In similar experiments, cytotoxicity was also observed with additional ACTR variants in both cell lines. Percent cytotoxicity was plotted as a function of antibody concentration and a nonlinear regression analysis was generated with GraphPad Prism and used to determine EC₅₀ values. The percent maximal cytotoxicity and EC₅₀ for these experiments can be found in Table 4, below. Additional ACTR variants comprising a CD28 costimulatory domain were also evaluated. ACTR variants containing a C28 costimulatory domain demonstrate a range of EC₅₀s with Raji target cells and rituximab (0.02-0.38 μg/mL) and with HCC1954 target cells and trastuzumab (0.003-0.09 μg/mL). These experiments demonstrate that the ACTR variants show antibody-dependent cytotoxicity against cell lines expressing the cognate target for the antibody.

TABLE 4
Cytotoxicity with different ACTR variants

| | Raji Target Cells + | | HCC1954 Target Cells + | |
|---|---|---|---|---|
| | Rituximab | | Trastuzumab | |
| | % Maximal | EC₅₀ | % Maximal | EC₅₀ |
| SEQ ID | Cytotoxicity | (μg/mL) | Cytotoxicity | (μg/mL) |
| 1 | 74 | 0.34 | 70 | 0.04 |
| 2 | 75 | 0.31 | 73 | 0.05 |
| 3 | 71 | 0.34 | 68 | 0.06 |
| 4 | 83 | 0.09 | 76 | 0.03 |
| 5 | 84 | n.d.* | 79 | 0.02 |
| 6 | 79 | 0.38 | 77 | 0.02 |
| 7 | 83 | 0.19 | 80 | 0.01 |
| 8 | 84 | 0.42 | 83 | 0.01 |
| 9 | 97 | 0.07 | 88 | 0.01 |
| 10 | 82 | 0.25 | 79 | 0.03 |
| 11 | 82 | 0.06 | 87 | 0.14 |
| 12 | 80 | 0.02 | 84 | 0.003 |
| 13 | 90 | 0.07 | 85 | 0.06 |
| 14 | 95 | 0.02 | 88 | 0.003 |
| 15 | 90 | 0.02 | 89 | 0.002 |
| 16 | 95 | 0.02 | 91 | 0.01 |
| 17 | 90 | 0.04 | 92 | 0.003 |
| 38/39** | 72 | 0.02 | 88 | 0.005 |

*n.d. = not determined
**ACTR construct SEQ ID NO: 38 with SEQ ID NO: 39 expressed in trans

### Example 2: IL-2 Cytokine Release by ACTR-T Cells in the Presence of Target Cells

Gamma-retroviruses were generated that encoded the ACTR variants in Table 3. These viruses were used to infect primary human T-cells, generating cells that expressed these ACTR variants on the surface of infected cells. These cells were subsequently used in IL-2 release assays with CD20-positive Raji target cells and CD20-targeting rituximab with HER2-positive HCC1954 target cells and HER2-targeting trastuzumab. Mock T-cells (T-cells not expressing ACTR variants) were used as controls in this experiment.

T-cells (effector; E) and Raji target cells (target; T) were incubated at a 4:1 effector-to-target ratio (120,000 effector cells; 30,000 target cells) in the presence of different concentrations of rituximab (0-10 μg/mL) in a 100-μL reaction volume in RPMI 1640 media supplemented with 10% fetal bovine serum. All reactions were carried out in duplicate. Reactions were incubated in a CO₂ (5%) incubator at 37 degrees C. for 20-24 hours.

In a separate experiment, T-cells (effector; E) and HCC1954 target cells (target; T) were incubated at a 1:4 effector-to-target ratio (30,000 effector cells; 120,000 target cells) in the presence of different concentrations of trastuzumab (0-1 μg/mL) in a 100-μL reaction volume in RPMI 1640 media supplemented with 10% fetal bovine serum. All reactions were carried out in duplicate. Reactions were incubated in a CO₂ (5%) incubator at 37 degrees C. for 20-24 hours.

For each experiment, the amount of released cytokine IL-2 was measured from the supernatants using the Meso Scale Discovery V-Plex Human IL-2 kits according to the manufacturer's protocol. Briefly, the Proinflammatory Panel 1 Calibrator Blend, SULFO-TAG Detection Antibody, and Read Buffer were prepared according to the manufacturer's protocol. Co-culture supernatants were thawed on ice and diluted in RP10 (RPMI 1640 supplemented with 10% fetal bovine serum) media to achieve values within the linear range of the assay. Proinflammatory calibrator blend or sample (50 μL) was then added to the MSD plate. The plate was subsequently sealed, covered in foil, and incubated on a room temperature shaker for two hours at 600×g. The plate was then washed three times with 150 L of phosphate buffered saline containing 0.05% Tween-20 (PBST) before Human IL-2 SULFO-TAG detection antibody (25 μL) was added to the plate. The plate was then sealed, covered in foil, and incubated on a room temperature shaker for two hours at 600×g. The plate was washed three times with 150 μL PBST. Read buffer (150 μL) was added to the plate and the plates were run on the MSD Quickplex SQ 120.

Raw data was analyzed in the MSD workbench using a plate layout created for the Single Plex IL-2 MSD kits. Standard curves were adjusted to match the kit lot for each plate analyzed. Raw data in light units was extrapolated to cytokine concentration (μg/mL) using the Proinflammatory calibrator standard curve. Cytokine data were plotted as a function of antibody concentration.

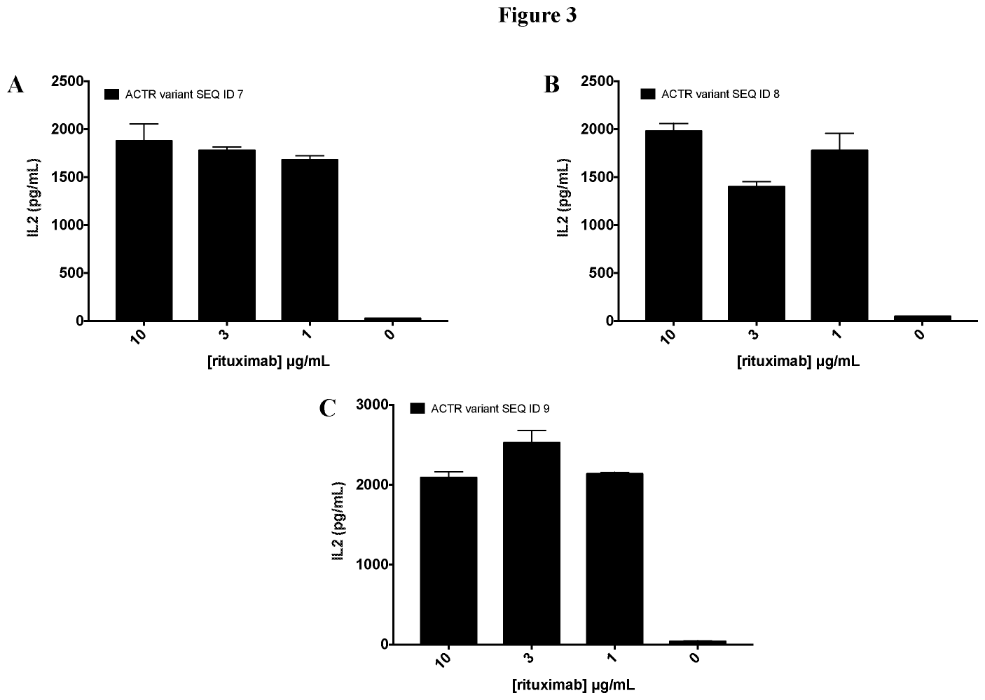

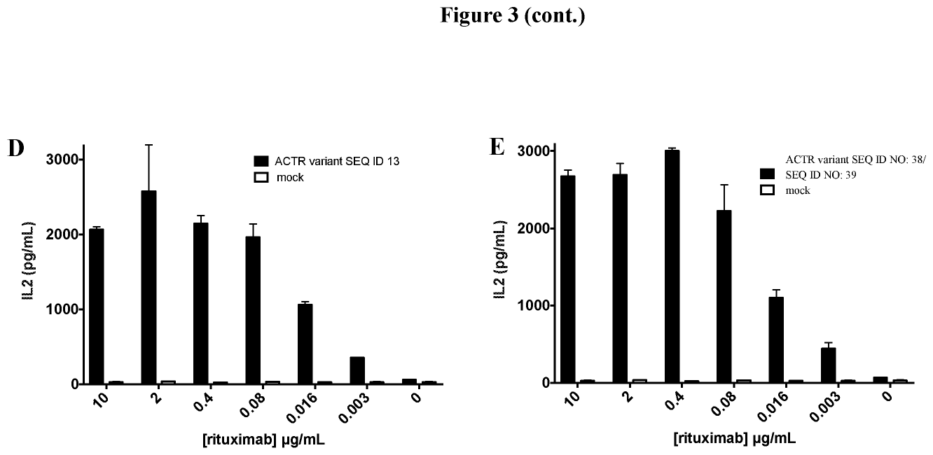

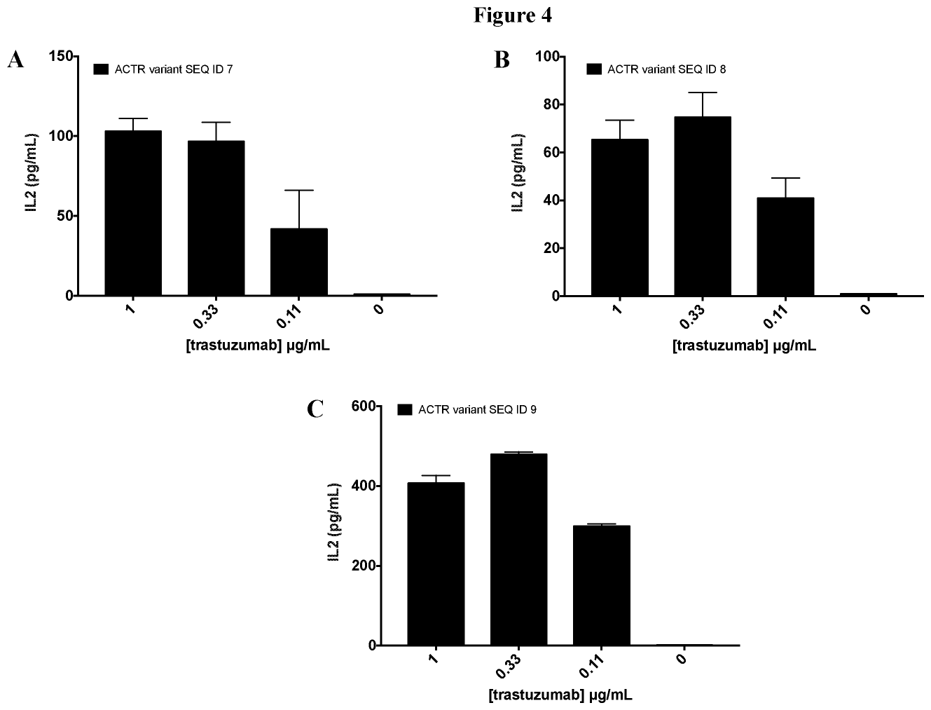

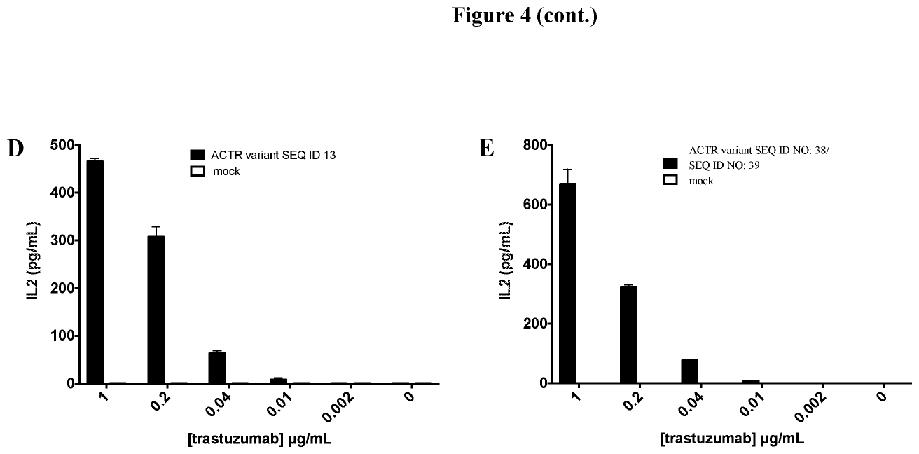

When target Raji cells were incubated with ACTR T-cells expressing nucleic acids encoding variants of SEQ ID NOs: 7, 8, 9, 13, and SEQ ID NO: 38/SEQ ID NO: 39 and increasing concentrations of rituximab, an antibody-dependent and concentration-dependent increase in IL-2 release was observed (FIG. 3). Similarly, when target HCC1954 cells were incubated with ACTR T-cells expressing nucleic acids encoding variants of SEQ ID NOs: 7, 8, 9, 13, and SEQ ID NO: 38/SEQ ID NO: 39 and increasing concentrations of trastuzumab, a concentration-dependent increase in IL-2 release was observed (FIG. 4). Mock T cells showed little or no IL-2 release. In similar experiments, IL-2 release was also observed with additional ACTR variants with both cell line/antibody pairs and the maximal IL-2 release from these experiments can be found in Table 5. ACTR variants containing a C28 costimulatory domain demonstrate a range of maximal IL-2 release in the presence of Raji target cells and rituximab (280-3600 μg/mL) and in the presence of HCC1954 target cells and trastuzumab (50-700 μg/mL). These experiments demonstrate that ACTR variants show antibody-dependent cytokine release in the presence of cell lines expressing the cognate target for the antibody.

TABLE 5
IL-2 release of T cells expressing ACTR variants

| | Maximal IL-2 Release (pg/mL) | |
|---|---|---|
| | | HCC1954 Target Cells + |
| SEQ ID | Raji Target Cells + Rituximab | Trastuzumab |
| 1 | 222 | 24.4 |
| 2 | 1846 | 196.6 |
| 3 | 308 | 30.83 |
| 4 | 453 | 23.54 |
| 5 | 663 | 56.85 |
| 6 | 282 | 86.36 |
| 7 | 1877 | 102.9 |
| 8 | 1980 | 74.63 |
| 9 | 2526 | 478.8 |
| 40/39* | 2290 | 165.7 |
| 11 | 1629 | 171 |
| 12 | 1034 | 146 |
| 13 | 3555 | 577 |
| 14 | 2815 | 315 |
| 15 | 1208 | 183 |
| 16 | 1194 | 3.5 |
| 17 | 2231 | 346 |
| 38/39** | 3003 | 669 |

*ACTR construct SEQ ID NO: 40 with SEQ ID NO: 39 expressed in trans
**ACTR construct SEQ ID NO: 38 with SEQ ID NO: 39 expressed in trans

### Example 3: Proliferation of ACTR-T Cells

Gamma-retroviruses were generated that encoded the ACTR variants in Table 3. These viruses were used to infect primary human T-cells, generating cells that expressed these ACTR variants on the surface of infected cells. These cells were subsequently used in proliferation assays with CD20-positive Raji target cells and CD20-targeting rituximab.

T-cells and target cells were mixed at a 1:1 ratio (30,000 cells each) in the presence of 4 μg/mL rituximab or with no added antibody in a 200-μL reaction volume in culture media (RPMI 1640 media supplemented with 10% fetal bovine serum). Reactions were incubated in a CO₂ (5%) incubator at 37° C. for 7 days; 70 μL of culture media was added to each reaction on day 4. Cells were pelleted by centrifugation, washed with phosphate-buffered saline (PBS), and stained with Fixable Viability Dye eFluor450 (eBioscience). Cells were washed twice with cell staining buffer containing bovine serum albumin and sodium azide (Biolegend). Cells were then stained with anti-CD16-Alexa-Fluor-647 (clone 3G8, Biolegend) and anti-CD3-Alexa-Fluor-488 (clone OKT3, Biolegend) antibodies. Cells were then washed twice in cell staining buffer before being resuspended in 200 μL of staining buffer.

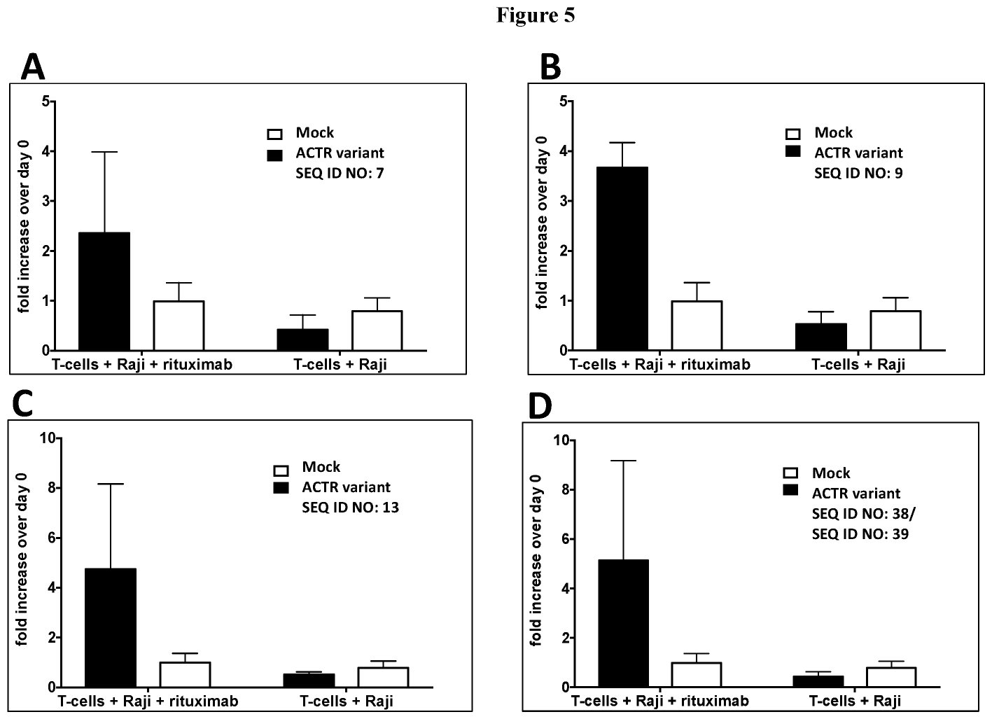

Stained cells were analyzed by flow cytometry using an Attune™ NxT Acoustic Focusing Cytometer. Singlet, live, CD3-positive T-cells were used to evaluate proliferation. The fold increase in cell count at day 7 relative to day 0 was plotted as a function of condition (FIG. 5). When target Raji cells were incubated with ACTR T-cells expressing nucleic acids encoding variants of SEQ ID NOs: 7, 8, 9, 13, and SEQ ID NO: 38/SEQ ID NO: 39 and rituximab, an antibody-dependent increase in the number of CD3+ cells was observed, indicating antibody-dependent T-cell proliferation (FIG. 5). In additional experiments, T-cell proliferation was evaluated in the presence of 5 μg/mL rituximab under conditions similar to those described above with T-cells expressing a number of different ACTR variants. The increase in the number of CD3+ cells relative to reactions without antibody was determined. These ACTR variants showed antibody-dependent proliferation (Table 6). Additional ACTR variants encoding CD28 costimulatory domains were also evaluated. ACTR variants containing a C28 costimulatory domain demonstrate a range increase in the number of CD3+ cells relative to reactions without antibody (2.5-15-fold). These experiments demonstrate that ACTR variants show antibody-dependent proliferation in the presence of cell lines expressing the cognate target for the antibody.

TABLE 6
Antibody-dependent proliferation of
T cells expressing different ACTR variants

| | Fold proliferation relative |
|---|---|
| SEQ ID | to no antibody |
| 1 | 3.41 |
| 2 | 3.84 |
| 3 | 2.36 |
| 4 | 3.35 |
| 5 | 4.81 |
| 6 | 3.91 |
| 7 | 3.34 |
| 8 | 2.56 |
| 9 | 4.28 |
| 40/39* | 2.51 |
| 11 | 4.28 |
| 12 | 2.99 |
| 13 | 14.98 |
| 14 | 5.14 |
| 15 | 3.26 |
| 16 | 6.44 |
| 17 | 6.49 |
| 38/39** | 10.42 |

*ACTR construct SEQ ID NO: 40 with SEQ ID NO: 39 expressed in trans
**ACTR construct SEQ ID NO: 38 with SEQ ID NO: 39 expressed in trans

### Example 4: Anti-Tumor Effects of ACTR-T Cells in Raji Tumor Animal Model

Raji Model

The Raji cell line (ATCC, catalog number CCL-86) is a human Burkitt's lymphoma cell line that forms tumors in immune compromised mice upon intravenous, intraperitoneal or subcutaneous implantation. For the experiments in this Example, Raji cells were transduced with Firefly luciferase using RediFect FFLuc-Puromycin (Perkin Elmer) lentiviral particles and maintained in RPMI-1640 medium supplemented with 10% heat-inactivated fetal bovine serum and 1 μg/mL puromycin in a 37 degree C., 5% CO₂-humidified chamber. Tumor cell viability was 98% upon harvest for inoculation.

Female NSG™ (NOD scid gamma, NOD.Cg-Prkdc^{scid} IL-2rg^{tm1Wjl}/SzJ, Strain 005557) mice, six weeks old, were obtained from The Jackson Laboratory (Bar Harbor, Me.). These mice, which lack functional T, B and NK cells, are particularly well suited for engraftment of human tumors and reconstitute efficiently with human T cells such as ACTR T cells. Upon receipt, the mice were acclimated for eight days prior to study initiation. The mice showed no signs of disease or illness upon arrival or prior to study initiation. The animals were housed five per cage in individually ventilated Innovive cages in a self-contained micro-isolator racks.

The intravenous Raji xenograft model was developed by inoculation of a suspension of luciferase-expressing Raji cells into female NSG™ mice. Raji cells (1×10⁵ cells per mouse) suspended in 0.1 mL DPBS were injected intravenously into the lateral tail vein. Mice developed detectable tumors within 6 days, primarily located to the bone marrow (spinal column, skull, long bones).

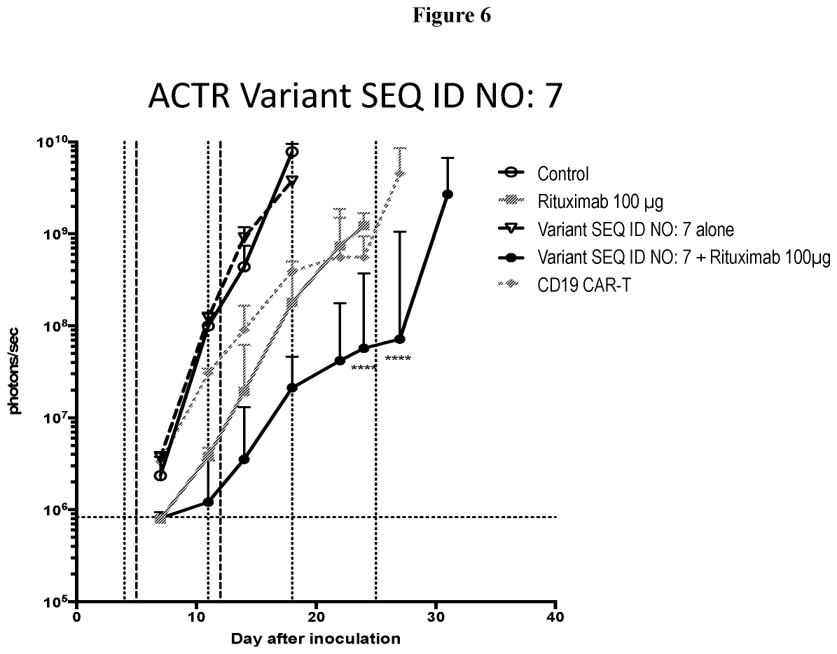

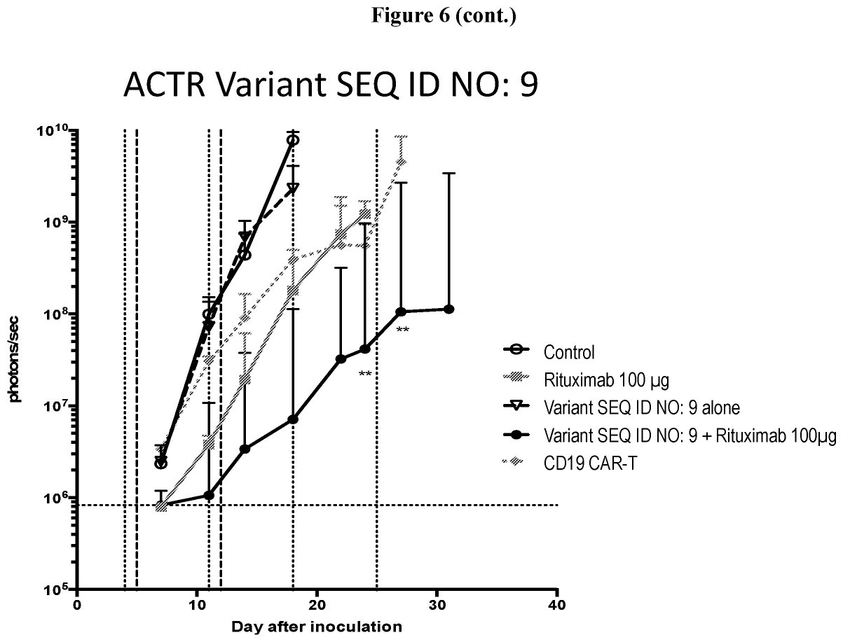

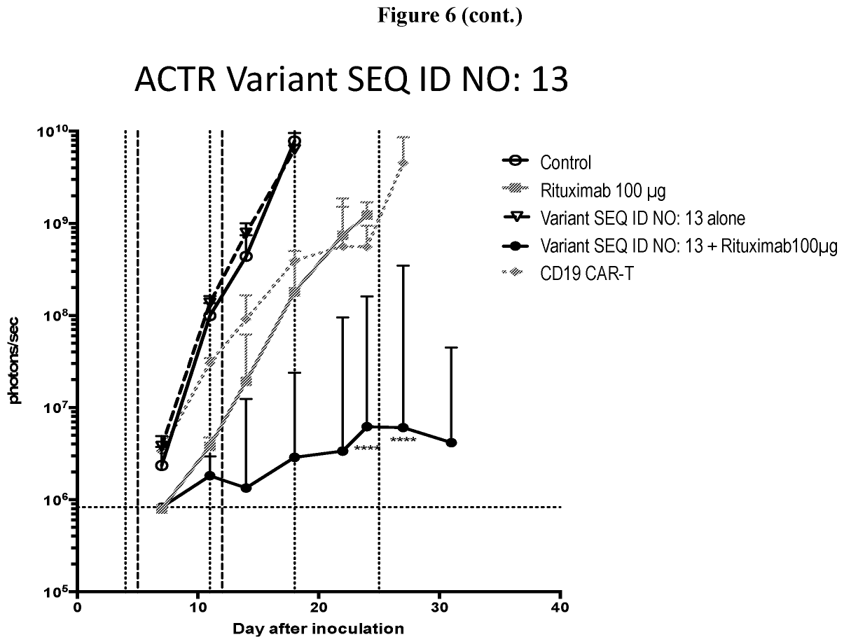

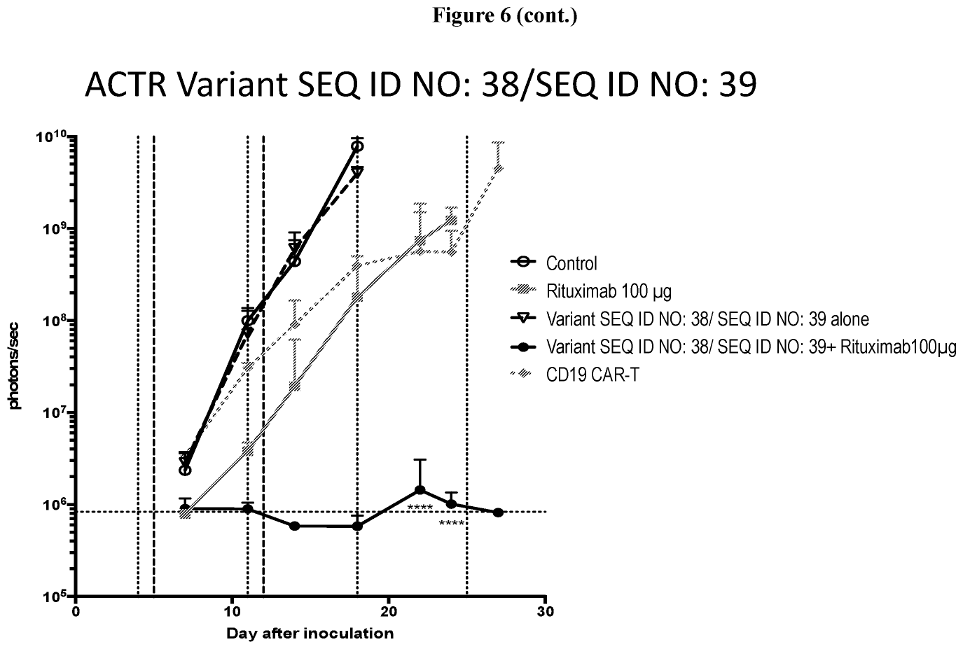

Mice were dosed intraperitoneally on Day 4, 11, 18 and 25 with 100 μg of rituximab (100 μL volume). On Day 5 and 12, mice were administered intravenously with 1×10⁷ T cells (T-cells expressing nucleic acids encoding ACTR variants SEQ ID NOs: 7, 9, 13, and SEQ ID NO: 38/SEQ ID NO: 39) in 100 μL. One group of mice were dosed with T cells expressing a CD19-targeting CAR (anti-CD19 scFv, CD8 hinge and transmembrane domain, 4-1BB costimulatory domain, CD3-zeta signaling domain); these mice were not dosed with rituximab. Beginning on Day 6 post cell inoculation, tumor burden was monitored by bioluminescence imaging twice weekly using the IVIS Spectrum imaging system (Perkin Elmer). Mice were weighed twice weekly in order to monitor their health. Mean photons/second were plotted versus time (FIG. 6). Euthanasia criteria were based on body weight loss or gain and clinical symptoms, particularly hind limb paralysis. Mice were followed for survival until study termination (Day 55).

Treatment groups included mice treated with vehicle control, with rituximab anti-CD20 antibody alone (Roche, 100 μg/mouse or 5 mg/kg), the ACTR variant alone, CD19 CAR, or a combination of rituximab and ACTR variant T cells.

Control mice succumbed from disease burden on Day 18 or 19. Rituximab treatment alone induced a decrease in tumor burden and extended survival of the mice to 24 days. Treatment with ACTR T cells alone did not provide a significant decrease in tumor burden or survival, regardless of the ACTR variant tested. Treatment with CD19 CAR resulted in a decrease in tumor burden similar to that observed with rituximab alone.

Treatment with ACTR variants in combination with rituximab resulted in greater tumor growth inhibition than vehicle control, ACTR variant alone, or rituximab alone. The differences over rituximab alone reached statistical significance (2-way ANOVA with Sidak's multiple comparison test) for all ACTR variants (7, p<0.0001; 9, p<0.01; 13, p<0.0001; and 18, p<0.0001) (FIG. 6).

### Example 5: Repeated Stimulation Proliferation Assay

ACTR-T cells or CD19 CAR T cells were generated and prepared in RPMI-10 media (RPMI1640+10% FBS) at 1×10⁶ cells/mL for the assay. ACTR and CAR expression was determined by flow cytometry (see below) and adjusted to 30% ACTR/CAR positive cells with donor-matched mock T-cells to normalize ACTR/CAR expression for all variants.

Raji tumor cells were harvested, counted, and adjusted to 2×10⁶ cells/mL with RPMI-10 media. Rituximab antibody was diluted to 4 μg/mL for a final antibody concentration in the assay of 1 μg/mL. T-cells (550 μL) were added into a 24 well plate, followed by 275 μL of the Raji cells for an effector:target (E:T) ratio of 1:1, and 275 μL of rituximab or media control. Cells were mixed well and a 70 μL aliquot taken for flow cytometry analysis (baseline counts). The plate was incubated for 3-4 days at 37 degrees C. to allow for target cell killing by the ACTR variant T cells or CAR-T cells and T cell proliferation.

On Day 3 or 4, an aliquot (70 μL) of each culture was removed and analyzed by flow cytometry for T cell and tumor cell counts. The remaining cultures were pelleted by centrifugation (500×g for 5 minutes) and resuspended in a predefined volume of fresh media. Cultures containing T cells that expanded during the 3-4-day incubation period were re-adjusted to approximately 1×10⁶ cells/mL and transferred to a 12-well plate. Non-proliferating cultures were resuspended in 1-1.25 mL of fresh media and maintained in a 24-well plate. Fresh Raji cells were prepared at 4×10⁶ cells/mL and an appropriate volume added to the mixed T cell/tumor cell cultures to bring the E:T cell ratio back to 1:1, if needed. Rituximab was added to a final concentration of 1 μg/mL to stimulated wells, while only media was added to control wells. Cultures were again incubated for 3-4 days. This restimulation process was repeated every 3-4 days for a total of 4 stimulation rounds.

On Day 14, all cultures were harvested and analyzed by flow cytometry for T cell (CD3+) and tumor cell (CD3−) counts. Cumulative T cell expansion and tumor killing was analyzed over time.

For flow cytometry analysis, each culture was mixed thoroughly, and 70 μL removed and transferred to a 96-well round-bottom polypropylene plate. Two aliquots of 25 L each were stained with anti-human CD3-AlexaFluor488 (T cell marker; Biolegend) and anti-human-CD16-AlexaFluor647 (ACTR marker; Clone 3G8, Biolegend) antibody in 150 L of MACS buffer (Miltenyi). Samples were mixed and incubated at 4 degrees C., in the dark, for 15 minutes. Samples were then incubated with a propidium iodide (PI) solution in L of MACS buffer and analyzed on a flow cytometer (Attune NxT). A 100-μL volume was acquired for each sample. Flow cytometry data analysis was performed using FlowJo (TreeStar). The live, singlet cell population was used to further determine the absolute count of live CD3+ T cells, and CD3-negative Raji cells.

The fold change in T cell number relative to the starting number of T cells was plotted as a function of time (FIG. 7). All tested ACTR variants proliferated in the presence of target cells and rituximab. All ACTR T cell variants out-proliferated the CD19 CAR-T cells except ACTR variant SEQ ID NO: 8, which was comparable to CD19 CAR-T cells. ACTR T cell variants expanded 3-8 fold over the 14-day assay period under rapid re-stimulation with antibody-opsonized target cells, with ACTR variant SEQ ID NO: 9 showing the greatest proliferation. ACTR T cells stimulated with target cells in the absence of antibody did not expand, and were overgrown by target cells (data not shown).

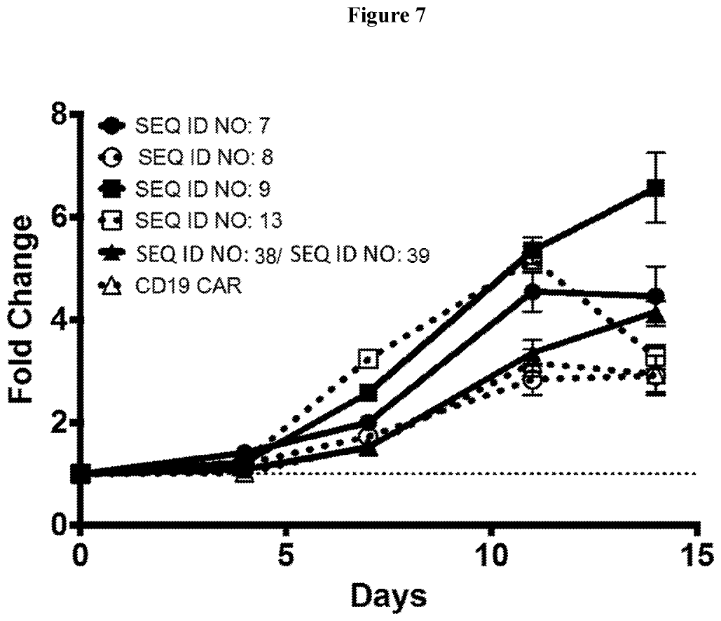

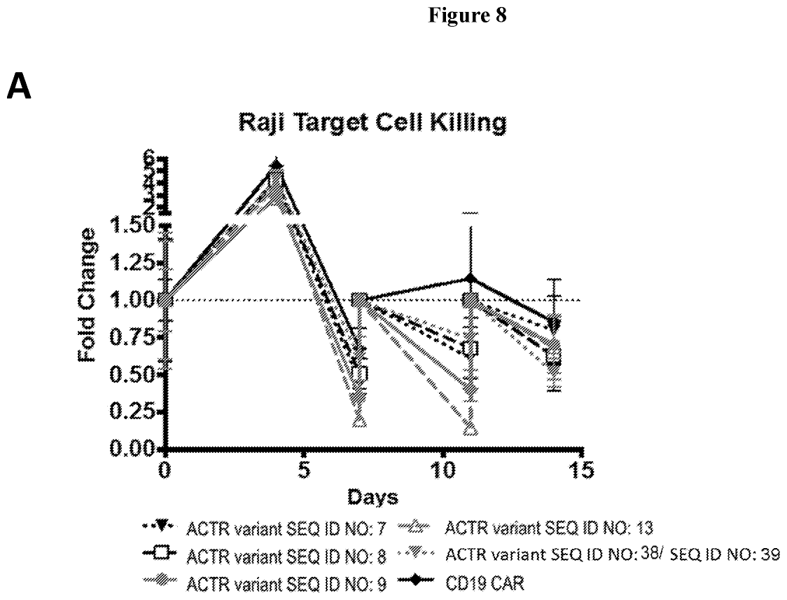

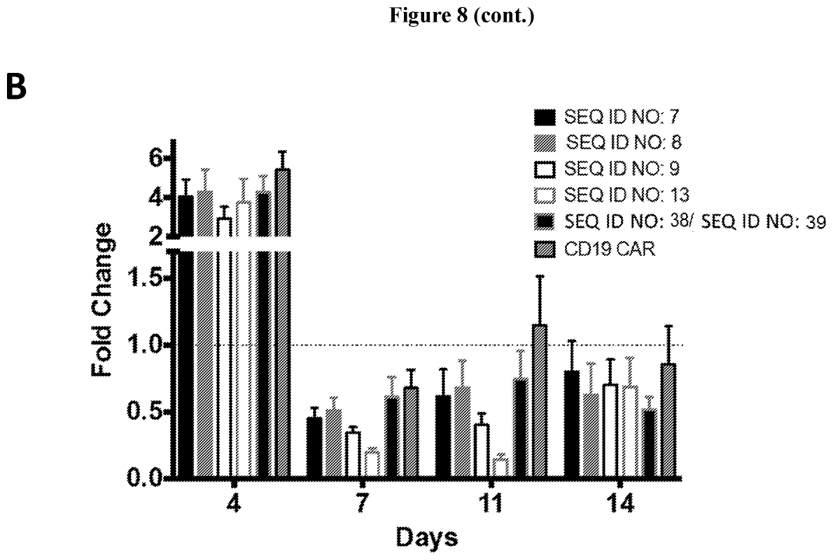

The fold change in Raji target cell number relative to the number of target cells at the previous time point were plotted as a function time (FIG. 8, panels A and B). Raji target cells continued to grow after the first stimulation. ACTR variants and CD19 CAR T cells controlled and killed Raji target cells to varying degrees starting after the second round of stimulation. Raji target cells continued to grow in the presence of ACTR variants in the absence of rituximab antibody (data not shown).

### Example 6: Use of T Cells Expressing ACTR Variant SEQ ID NO: 9 in Combination with Rituximab In Vitro and In Vivo with CD20-Expressing Tumor Cell Lines

This example demonstrates that T cells expressing the ACTR variant SEQ ID NO: 9 (referred to as ACTR throughout this example) successfully mediated anti-tumor cell activities in vitro and tumor regressions of aggressive CD20+ lymphomas in vivo when combined with rituximab. Details and results of experiments using this construct are presented below.

Initial Testing of ACTR

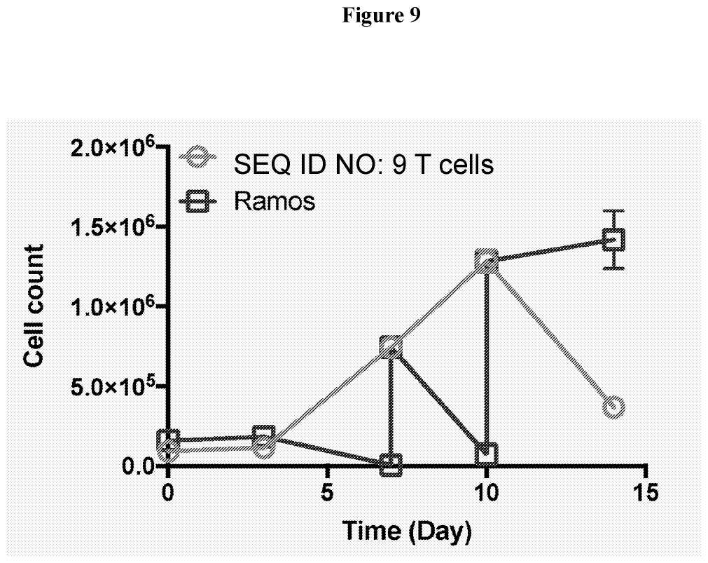

Activities of T cells expressing ACTR were analyzed in a repeated simulation “stress test” where ACTR variant expressing T cells were challenged with fresh CD20+ Ramos tumor cells and rituximab every three days with a procedure similar to that described in Example 5. ACTR T cells continued to proliferate and were cytotoxic to target Ramos cells after three repeated stimulations and started to lose proliferative and cytotoxic capacity after a fourth stimulation (FIG. 9).

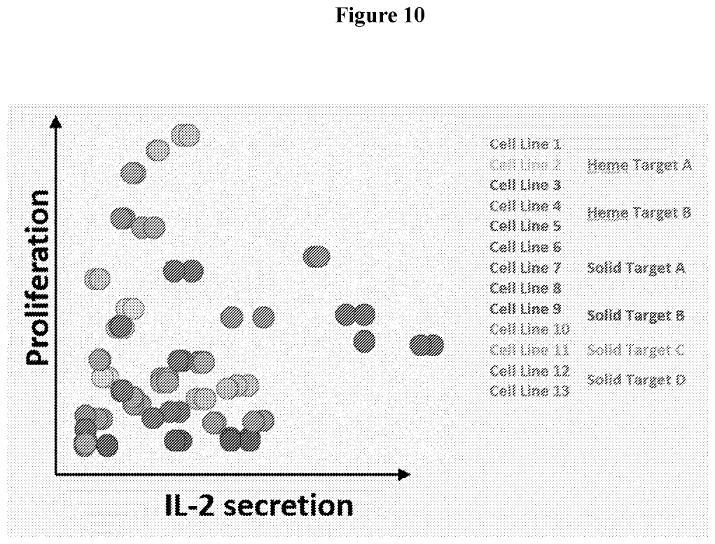

Cytokine release (IL-2) and proliferation assays were performed in a manner similar to that described in Examples 2 and 3 with antibodies specific to six different targets expressed in the hematologic and solid tumor disease settings and cell lines expressing the cognate target. T cell activity, as measured by both IL-2 release and proliferation, was demonstrated across a plurality of cells lines representing different indications (FIG. 10).

Rituximab Binds CD20+ Tumor Cells and ACTR T Cells

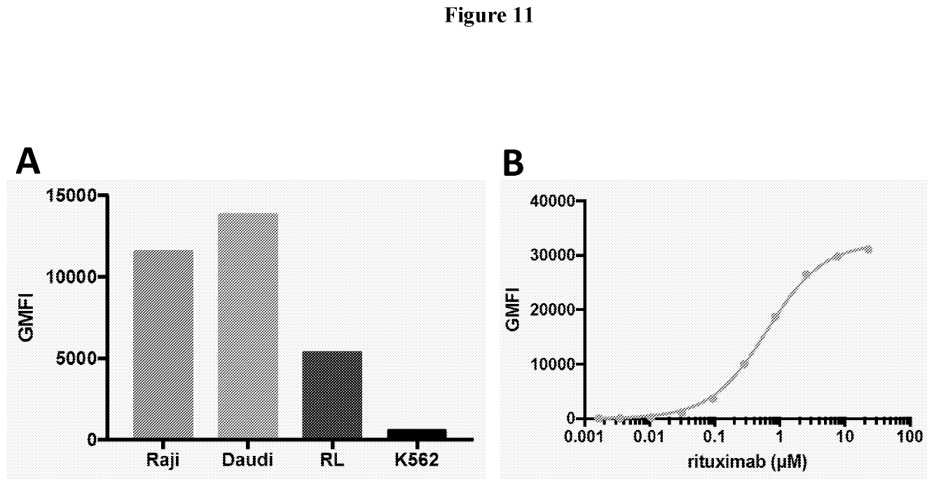

Rituximab binding to Raji, Daudi, and RL CD20+ lymphoma tumor cells was analyzed by flow cytometry by staining cells with rituximab and a fluorochrome-labeled anti-human Fc detection antibody (FIG. 11, panel A). No specific binding was observed for the CD20-negative cell line K562. The ability of rituximab to bind to ACTR T cells was also evaluated by incubating different concentrations of rituximab with ACTR-expressing T cells. Bound rituximab was detected with a fluorochrome-labeled anti-human Fc antibody and analyzed by flow cytometry. Rituximab binds to ACTR-expressing T cells in a concentration-dependent manner (FIG. 11, panel B). The apparent affinity of rituximab for ACTR T cells is 689±82 nM.

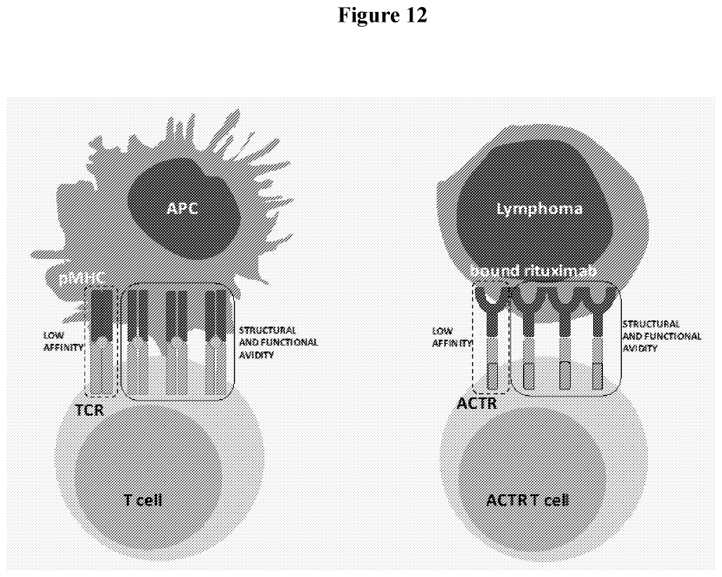

Without being bound by theory, a hypothetical model for a suggested mechanism of action of ACTR-T cell activation is shown in FIG. 12. Similar to low-affinity natural T cell receptors and Fc receptors, ACTR T cells may be activated via structural avidity when ACTR engages multiple rituximab molecules bound to the surface of tumor cells.

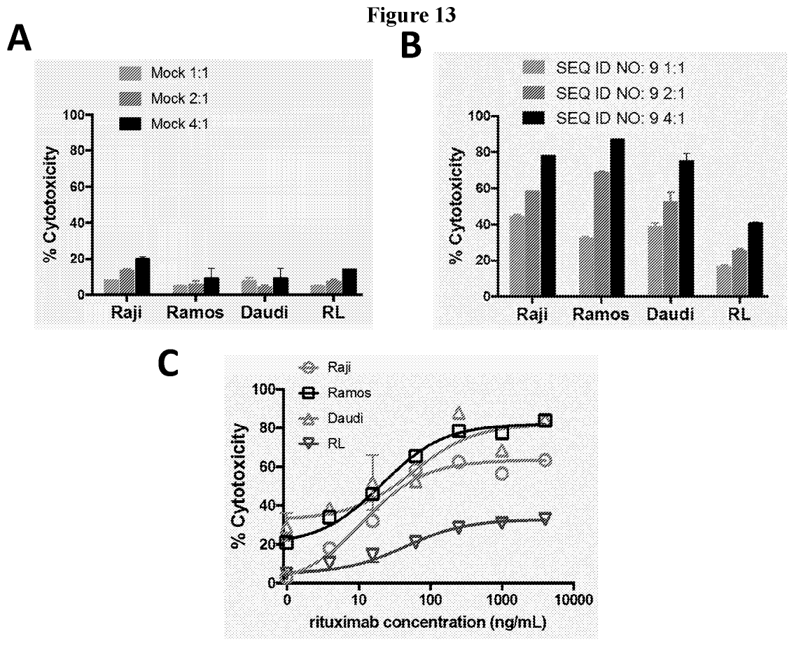

Cytotoxicity assays were carried out with mock (no ACTR) and ACTR-expressing T cells and CD20+ tumor cells in the presence of 1 μg/mL rituximab. T cells and target cells were mixed at different effector (E) to target (T) ratios and incubated at 37 degrees C. in a 5% CO₂ incubator for 24 hours. Reactions were stained with a cell viability dye, anti-human CD3, and anti-human CD19 antibodies and analyzed by flow cytometry. The percent cytotoxicity was determined by dividing the number of live, CD19+ cells in reactions with T cells, target cells, and rituximab by the number of live, CD19+ cells in control reactions without T cells, subtracting the resulting value from 1, and then multiplying by 100. Percent cytotoxicity is plotted as a function of target cell for different E:T ratios with mock cells (FIG. 13, panel A) and ACTR-expressing T cells (FIG. 13, panel B). The results of these experiments demonstrated that robust cytotoxicity is ACTR-dependent, as little or no cytotoxicity is observed with mock cells, and that cytotoxicity is dependent on ACTR T cell dose (FIG. 13, panels A and B). Similar experiments were carried out with ACTR T cells and CD20+ target cells at a 2:1 E:T ratio with varying concentrations of rituximab. Supernatants were removed from these reactions for cytokine analysis. ACTR T cells mediate rituximab-dose-dependent cytotoxicity of CD20+ tumor cells (FIG. 13, panel C).

Supernatants were analyzed for IFN-γ and IL-2 using the Meso Scale Discovery V-Plex Human IFN-γ and the V-Plex Human IL-2 kit according to the manufacturer's protocol. Briefly, the Proinflammatory Panel 1 Calibrator Blend, SULFO-TAG Detection Antibody, and Read Buffer were prepared according to the manufacturer's protocol. Co-culture supernatants were thawed on ice and diluted in RP10 (RPMI 1640 with 10% fetal bovine serum) media to achieve values within the linear range of the assay. Proinflammatory calibrator blend or sample (50 μL) was added to the MSD plate. The plate was sealed, covered in foil, and incubated on a room temperature shaker for two hours at 600×g. The plate was washed three times with 150 μL phosphate buffered saline containing 0.05% Tween-20. Human IFN-γ or human IL-2 SULFO-TAG detection antibody (25 μL) was added to the plate. The plate was sealed, covered in foil, and incubated on a room temperature shaker for two hours at 600×g. The plate was washed three times with 150 μL phosphate buffered saline containing 0.05% Tween-20. Read buffer (150 μL) was added to the plate and the plates were run on the MSD Quickplex SQ 120.

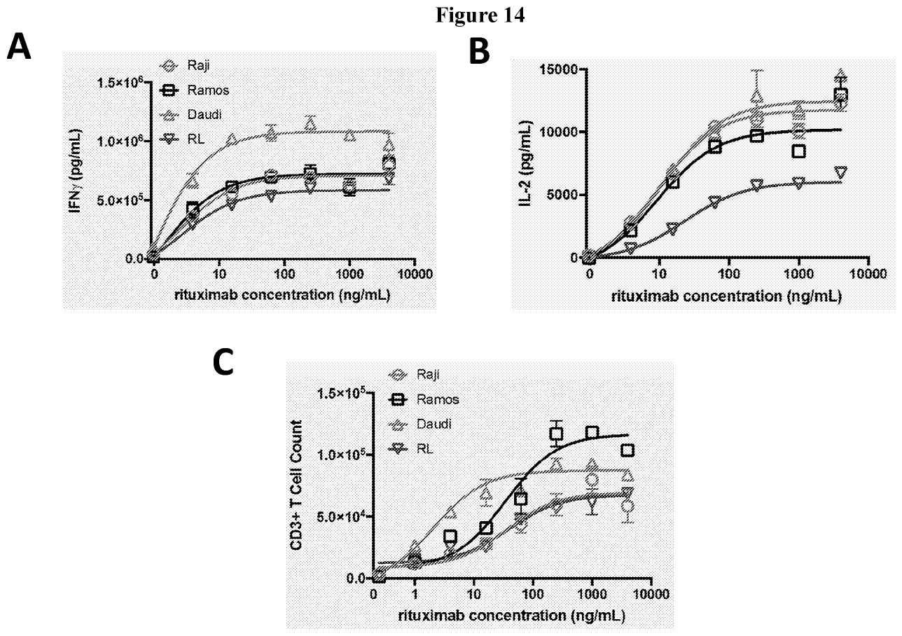

Raw data was analyzed in the MSD workbench using a plate layout created for Single Plex IFN-γ and Single Plex IL-2 MSD kits. Standard curves were adjusted to match the kit lot for each plate analyzed. Raw data in light units was extrapolated to cytokine concentration (pg/mL) using the Proinflammatory calibrator standard curve. Cytokine data were plotted as a function of antibody concentration (FIG. 14, panels A and B). Rituximab-concentration dependent IL-2 and IFN-γ cytokine release was observed with ACTR-expressing T cells.

Proliferation assays were carried out by incubating ACTR-expressing T cells and CD20+ target cells (Raji, Ramos, Daudi, RL) at a 1:1 E:T ratio in the presence of increasing concentrations of rituximab for 7 days at 37 degrees C. in a 5% CO₂ incubator. Cells were stained with a viability dye and anti-CD3 antibody and analyzed by flow cytometry. Live, CD3+ cells are plotted as a function of rituximab concentration (FIG. 14, panel C). ACTR expressing T cells were shown to proliferate in a rituximab-dependent and antibody-concentration-dependent manner.

Specificity of ACTR Activity

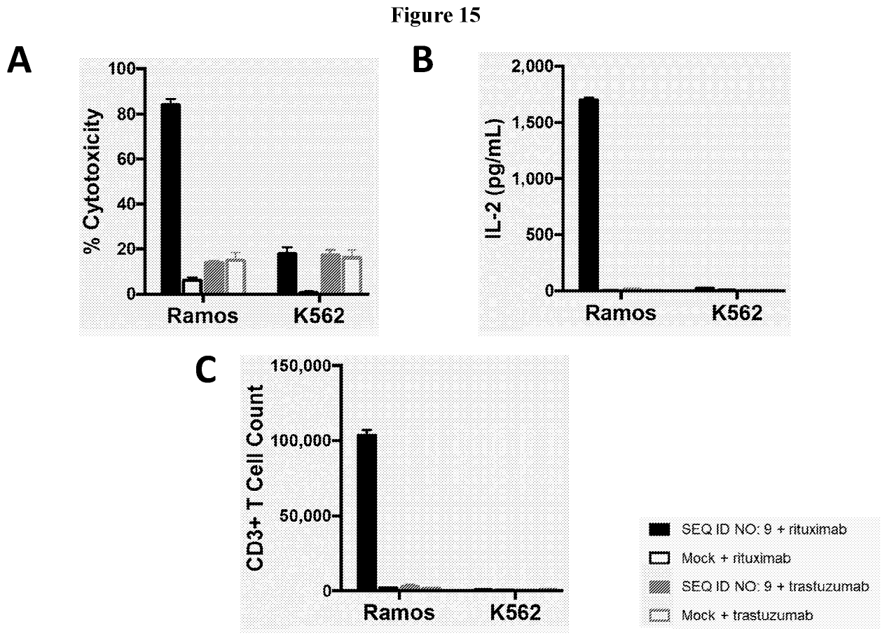

Cytotoxicity, IL-2 production, and T cell proliferation assays were carried out as described above with mock or ACTR T cells in the presence of CD20+ Ramos cells or CD20-negative K562 cells and either CD20-targeting rituximab or HER2-targeting trastuzumab; both Ramos and K562 cells are negative for HER2. Cytotoxicity and IL-2 release experiments were carried out at a 2:1 E:T ratio and proliferation experiments were carried out at a 1:1 E:T ratio. Antibodies were used at 4 μg/mL final concentration. The results of these experiments are shown in FIG. 15 panels A-C. Cytotoxicity, IL-2 release, and proliferation were observed in the presence of ACTR-expressing T cells, rituximab, and CD20+ cells; little to no T cell activity was observed under any of the other reaction conditions tested. These experiments demonstrate that ACTR T cell activity is dependent on ACTR expression and a target-bound antibody.

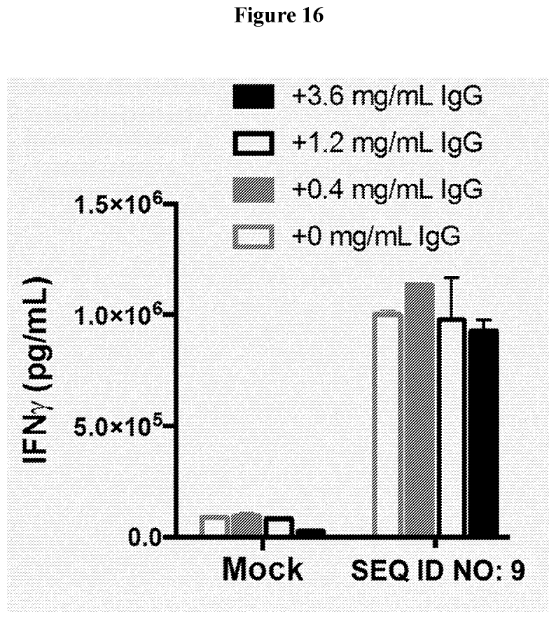

ACTR activity was also evaluated in the presence of non-targeting IgG. In these experiments mock or ACTR-expressing T cells were mixed at a 4:1 E:T ratio with CD20-expressing Raji cells. Cells were incubated with 1 μg/mL rituximab and increasing concentrations of non-targeting human IgG in serum (0-3.6 mg/mL). IgG was titrated by mixing pooled human AB serum and IgG-depleted serum to achieve different final IgG concentrations in the reactions. Reactions were incubated for 24 hr at 37 degrees C. in a 5% CO₂ incubator. IFN-γ release was assayed as described above. Mock T cells showed little or 10 no IFN-γ release; ACTR T cells showed robust IFN-γ release, which was similar across all non-targeting IgG concentrations tested (FIG. 16). These experiments demonstrate that non-targeting IgG has little to no impact on antibody-targeted ACTR activity.

Activation of ACTR-T Cells in the Presence of Various Antibodies

Tocilizumab is an anti-IL6 receptor (IL-6R) antibody that is used in T cell therapy treatment regimens to mitigate the effects of cytokine release syndrome (CRS). ACTR engagement of tocilizumab in the presence of IL-6R-expressing cells could exacerbate toxicities associated with CRS. These experiments demonstrate that ACTR-expressing T cells do not productively engage with tocilizumab in the presence of IL-6R-expressing cells.

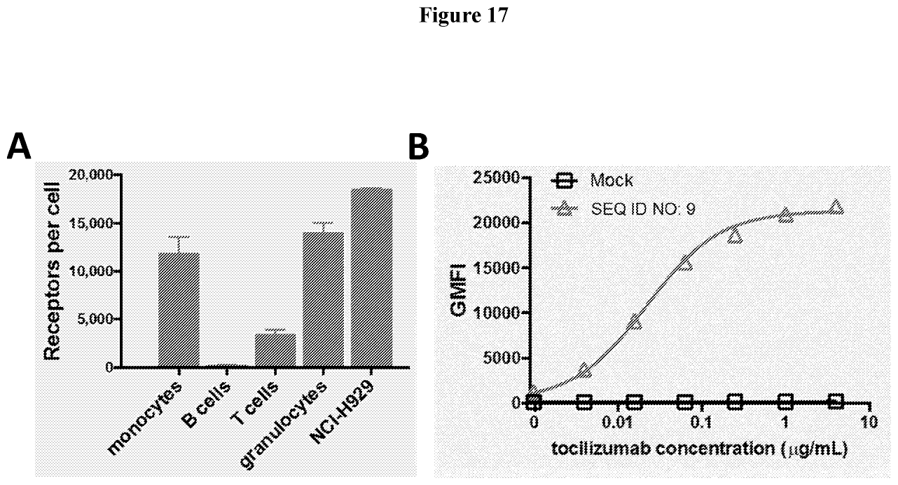

Receptor quantification of IL-6R on normal immune cells and the multiple myeloma cell line NCI-H929 was determined by flow cytometry (FIG. 17, panel A). These experiments demonstrate that NCI-H929 cells express comparable or greater number of IL-6R per cell relative to normal immune cells. A binding assay with tocilizumab was carried out in a manner similar to that described above for rituximab binding to ACTR-expressing T cells. Tocilizumab binds to ACTR-expressing T cells in a dose-dependent manner, while mock T cells that do not express ACTR showed no binding to tocilizumab (FIG. 17, panel B).

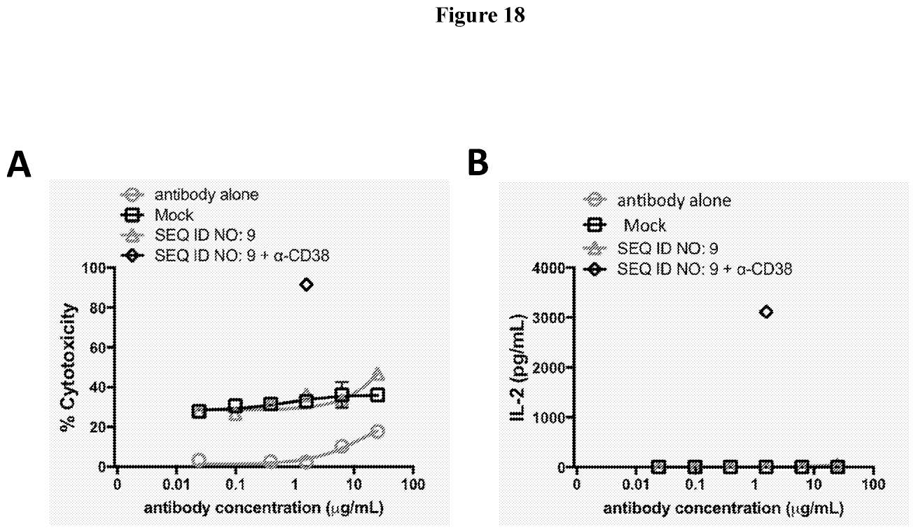

Cytotoxicity and IL-2 release assays were performed as described above. For these experiments mock or ACTR-expressing T cells were incubated with NCI-H929 cells at a 4:1 E:T ratio in the presence of increasing concentrations of tocilizumab (0-25 μg/L). NCI-H929 cells were also incubated with increasing concentrations of tocilizumab in the absence of T cells. A control experiment was carried out with ACTR T cells, NCI-H929 target cells, and the anti-CD38 antibody daratumumab (1.5 μg/mL); NCI-H929 cells express CD38. The results of these experiments demonstrated that ACTR T cells did not mediate cytotoxicity in the presence of IL-6R+NCI-H929 cells and increasing concentrations of tocilizumab above that observed with mock T cells or in the absence of T cells (FIG. 18, panel A). The results of these experiments also demonstrated that ACTR T cells did not mediate IL-2 release in the presence of IL-6R+NCI-H929 cells and increasing concentrations of tocilizumab, similar to what is observed with mock T cells or in the absence of T cells (FIG. 18, panel B).

ACTR T cells were shown to mediate cytotoxicity and cytokine release in the presence of NCI-H929 cells and the anti-CD38 antibody daratumumab in control experiments under the same conditions.

In Vivo ACTR Activity

In another experiment, the anti-tumor efficacy of ACTR T cells was assayed in an aggressive Raji xenograft in NSG™ (NOD scid gamma, NOD.Cg-Prkdc^{scid} IL-2rg^{tm1Wlj}/SzJ, Strain 005557) mice. Raji-luc cells were thawed for the study and grown in media for a limited number of passages. Raji-luc cells (1×10⁵ cells per mouse in 0.1 mL serum-free media) were injected intravenously into the lateral tail vein of female NSG mice. On Day 4 post cell inoculation, mice were randomized into treatment groups (n=5). Individual mice in each group were identified by ear puncture. Beginning on Day 6 post cell inoculation, tumor burden was monitored by bioluminescence imaging using the IVIS Spectrum (Perkin Elmer). Body weights of the mice ranged from 17.1 to 23.5 (20.5+/−1.3, Mean+/−SD) grams. Mean tumor radiance (photons/second) and body weights (grams) were measured over time and euthanasia criteria were based on body weight loss and clinical symptoms, but not on a bioluminescence cut-off. All animal experiments were performed in accordance with protocols approved by the Unum Institutional Animal Care and Use Committee (IACUC). All procedures were carried out in accordance with the Guide for the Care and Use of Laboratory Animals (National Research Council).

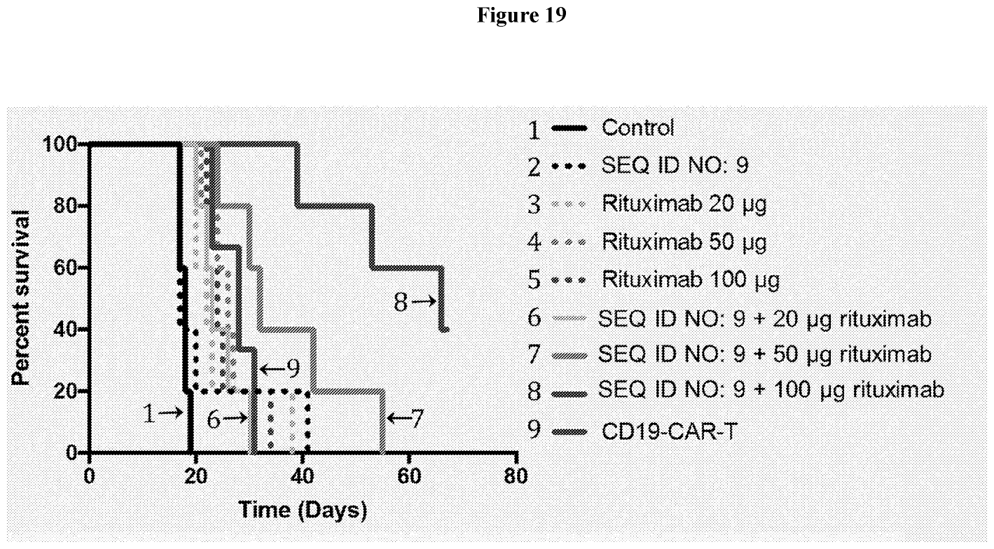

The experimental groups were no treatment (control), ACTR alone, rituximab alone at 20 μg, 50 μg, or 100 μg per dose, and ACTR+rituximab at 20 μg, 50 μg, and 100 μg rituximab per dose. T cells expressing CD19 CAR (anti-CD19 scFv linked to a 4-1BB costimulatory domain and a CD3-zeta signaling domain) were also evaluated in this experiment. In groups receiving rituximab, antibody was first dosed 4 days after tumor cell inoculation and was dosed weekly for a total of 4 doses. In groups receiving ACTR T cells, a single dose of 1×10⁷ ACTR T cells was given on day 5, one day following the first rituximab dose. The percent survival of treated mice was plotted as a function of time across all of the treatment groups (FIG. 19). The anti-tumor efficacy was potent and rituximab dose-dependent (FIG. 19).

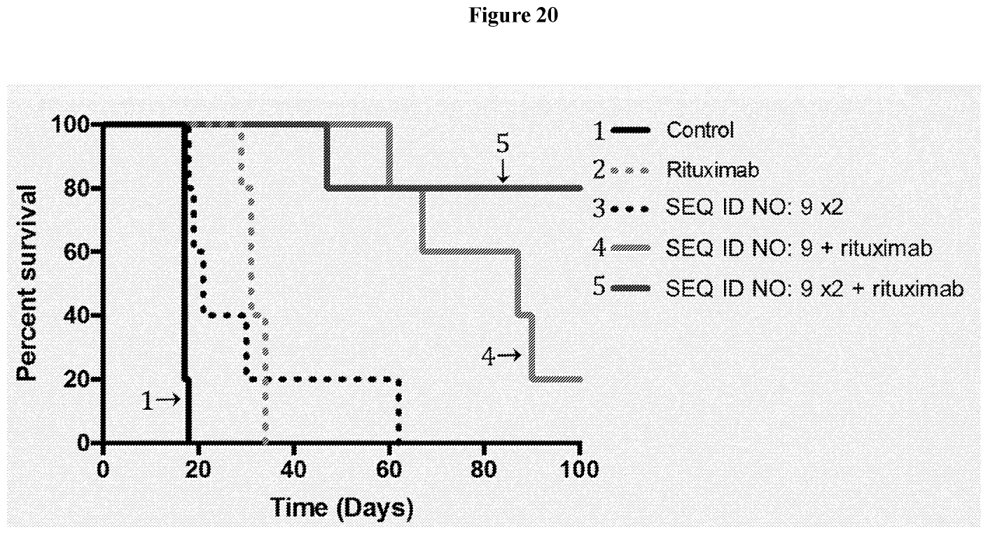

A similar experiment demonstrated that ACTR+rituximab anti-tumor efficacy was ACTR T cell dose-dependent. The experimental groups were no treatment (control), rituximab alone, ACTR alone (2 doses), ACTR (1 dose)+rituximab, and ACTR (2 doses)+rituximab. In groups receiving rituximab, antibody was first dosed 4 days after tumor cell inoculation and was dosed weekly for a total of 4 doses. In groups receiving ACTR T cells, a single dose of 1×10⁷ ACTR T cells was given on day 5, one day following the first rituximab dose; groups receiving two doses of ACTR T cells were treated again with 1×10⁷ ACTR T cells on day 12. Percent survival was plotted as a function of time (FIG. 20). Groups treated with both ACTR and rituximab showed robust anti-tumor activity as evidenced by enhanced survival. Survival was greater for the group treated with two doses of ACTR relative the group treated with one dose of ACTR, indicating that ACTR activity is dose-dependent.

ACTR Activity in T Cells Made from Non-Hodgkin Lymphoma (NHL) Donors

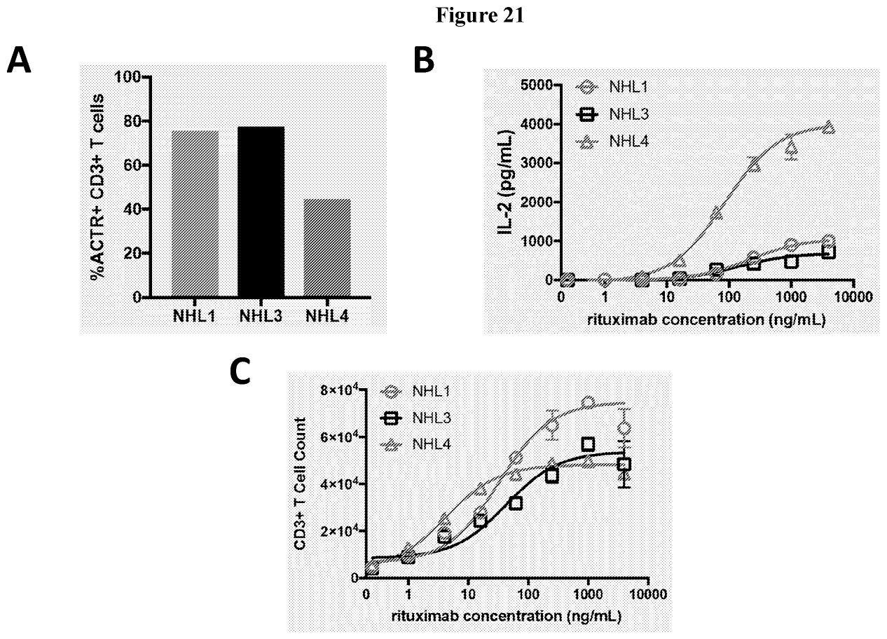

Gamma retrovirus encoding ACTR was tranduced into T cells generated from PBMCs of three unique Non-Hodgkin Lymphoma (NHL) donors and surface expression of ACTR on the T cells was confirmed by flow cytometry using an anti-CD16 detection antibody (FIG. 21, panel A). IL-2 release and proliferation experiments were carried out in a manner similar to that described above with Raji target cells and a 1:1 E:T ratio in the presence of increasing concentrations of rituximab. ACTR T cells generated from NHL donors were shown to mediate rituximab concentration-dependent IL-2 release (FIG. 21, panel B) and proliferation (FIG. 21, panel C).

Summary of Results

When combined with rituximab, ACTR expressing T cells displayed potent activation, proliferation, cytokine production, and tumor-directed cytotoxicity in the presence of CD20+ lymphoma cell lines. ACTR expressing T cell in vitro activity was dependent on rituximab and was dose-titratable. ACTR expressing T cells had potent anti-tumor efficacy in vivo in an aggressive Raji lymphoma xenograft model in NSG mice. Such anti-tumor activity was ACTR T cell and rituximab dose-dependent, with higher T cell numbers and antibody concentrations mediating improved responses. Taken together, these data demonstrate the specificity and versatility of the ACTR expressing T cell therapeutic approach to target diverse cancer antigens.

### Example 7: Effective Targeting of HER2-Amplified Cancers with Trastuzumab in Combination with T Cells Expressing ACTR Variant SEQ ID NO: 9

HER2 Expression on Tested Cell Lines

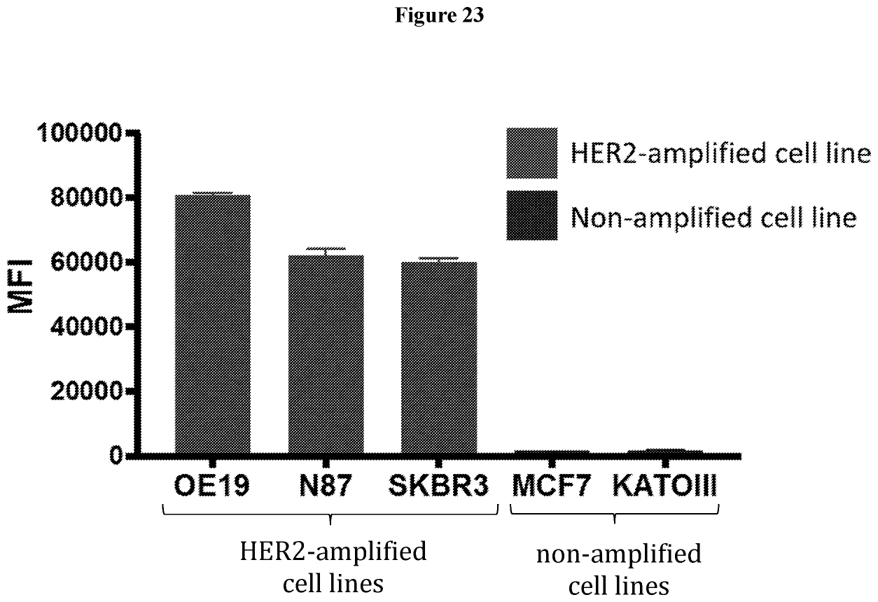

HER2 protein expression on HER2-amplified cell lines (OE19, N87, and SKBR3) and non-HER2-amplified cell lines (MCF7 and KATOIII) was evaluated by flow cytometry after staining with an anti-human HER2 antibody. Mean fluorescence intensity (MFI) of stained cells is plotted for each cell line evaluated (FIG. 23). HER2-amplified cell lines showed robust staining with the anti-HER2 antibody while minimal staining was observed with the non-HER2-amplified cells. These results are consistent with reported copy number data for these cell lines (see below).

The HER2 copy number (log₂) of various cell lines as well as their tissue origins is shown in Table 7. The HER2 gene is amplified in the OE19, N87 and SKBR3 cell lines, and is not amplified in the MCF7 and KatoIII cell lines. HER2 copy number was described in the Cancer Cell Line Encyclopedia (CCLE), which is incorporated by reference for its description of such cell lines.

TABLE 7
HER2 gene expression on amplified and non-amplified cell lines

| | | HER2 copy number |
|---|---|---|
| Cell Line | Tissue | (log₂) |
| OE19 | esophagus | 5.3 |
| N87 | gastric | 4 |
| SKBR3 | breast | 2.8 |
| MCF7 | breast | −0.96 |
| Kato III | gastric | 0.3 |

ACTR in Combination with Trastuzumab has Selective In Vitro Activity on HER2-Amplified Tumor Cell Lines

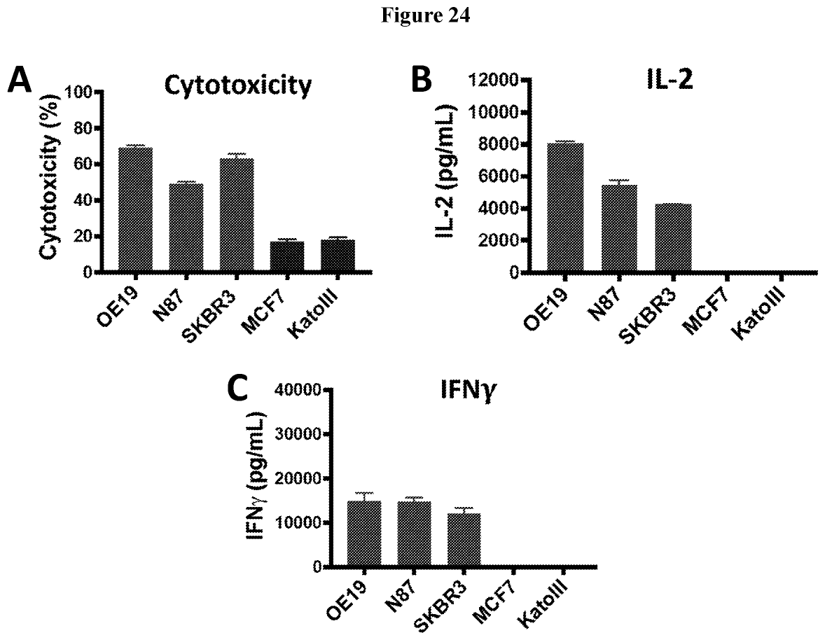

Cytotoxicity, IL-2 release, and IFN-γ release were evaluated with ACTR T cells and HER2-amplified cell lines (OE19, N87, and SKBR3) and non-HER2-amplified cell lines (MCF7 and KATOIII). Reactions were carried out at a 2:1 E:T ratio in the presence of 5 μg/mL anti-HER2 antibody trastuzumab at 37 degrees C. in a 5% CO₂ incubator in a 200-μL reaction volume. Supernatant was removed after 24 hours and analyzed for IL-2 and IFN-γ using a homogenous time resolved fluorescence (HTRF) assay (Cisbio). Briefly, the cytokine standards and conjugates were prepared according to the manufacturer's protocol. In a low volume 384-well plate, a 10-μL volume of conjugate and 10-μL volume of cell supernatant (1:4 diluted for IL-2, 1:16 diluted for IFN-γ) were co-incubated for 24 hours. The fluorescence signal was measured using an EnVision Multi-label plate reader and data was analyzed according to the manufacturer's recommendations. After 48 hours, cytotoxicity was evaluated using ATPlite one step (Perkin Elmer) as a measure of live target cells. Previous experiments demonstrated minimal contribution of T cells to the ATPlite signal. The percent cytotoxicity was determined by comparing the ATPlite signal in wells with ACTR and antibody to control wells in the absence of antibody. These experiments demonstrate that ACTR T cells+trastuzumab show robust cytotoxicity, IL-2 release, and IFN-γ release in the presence of the HER2-amplified cell lines OE19, N87, and SKBR3 but not in the presence of non-amplified cell lines MCF7 and KatoIII (FIG. 24).

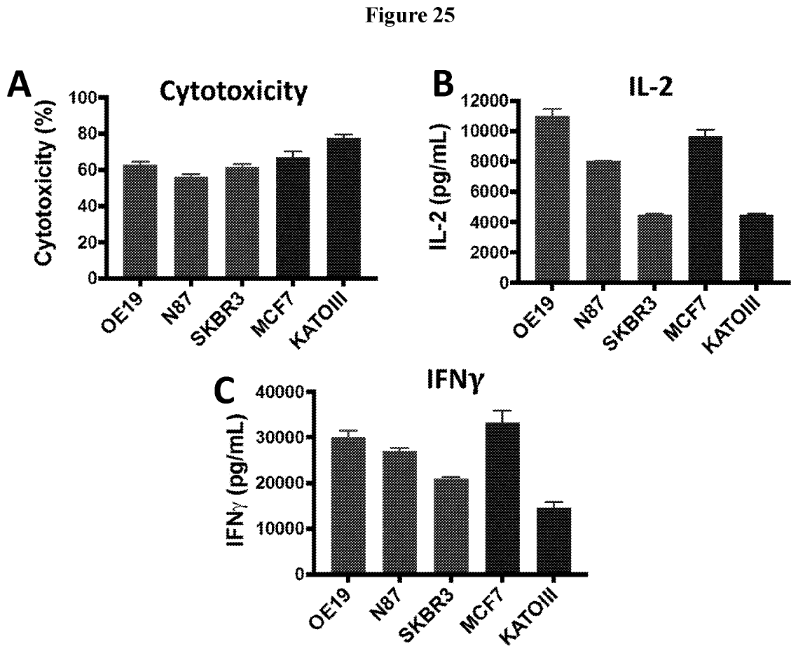

Similar experiments were carried out with T cells expressing an anti-HER2 CAR. This CAR variant was comprised of an scFv derived from the trastuzumab sequence (4D5), a CD8 hinge and transmembrane domain, a CD28 costimulatory domain, and a CD3-zeta signaling domain. Cytotoxicity and cytokine release experiments were carried out as described above with a 2:1 E:T ratio in the absence of trastuzumab. These experiments demonstrate that anti-HER2 CAR T cells mediate robust cytotoxicity, IL-2 release, and IFN-y release in the presence of the HER2-amplified cell lines OE19, N87, and SKBR3 and in the presence of non-amplified cell lines MCF7 and KatoIII (FIG. 25).

These experiments demonstrate that ACTR T cells+trastuzumab show selective activity against HER2-amplified cell lines while anti-HER2 CAR T cells do not show selective activity as a function of HER2 expression on target cells.

ACTR T Cells and HER2-Targeting CAR-T Cells Proliferate on HER2-Amplified Tumor Cell Lines

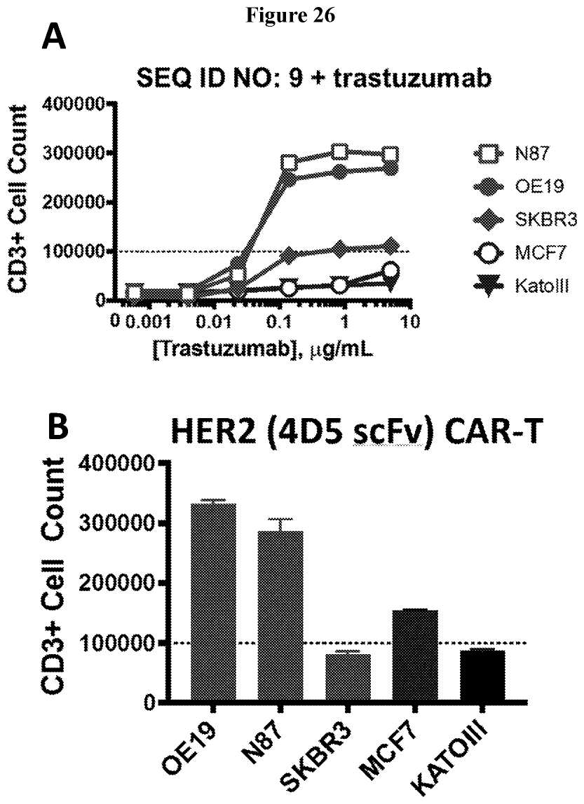

Proliferation assays were carried out in a manner similar to that described in Example 6 at a 2:1 E:T ratio for 6 days, starting with 100,000 T cells per well. Experiments with ACTR T cells were carried out with increasing concentrations of trastuzumab (0-5 μg/mL); experiments with anti-HER2 CAR T cells were carried out in the absence of trastuzmab. T cell proliferation was evaluated in the presence of HER2-amplified cell lines (OE19, N87, and SKBR3) and non-HER2-amplified cell lines (MCF7 and KATOIII). The results of these experiments demonstrated that ACTR T cells demonstrate trastuzumab dose-dependent proliferation in the presence of the HER2-amplified cell lines, N87 and OE19 (FIG. 26, panel A); and HER2-targeting CAR-T cells have comparable proliferation on N87 and OE19 target cells (FIG. 26, panel B). Little or no proliferation was observed with SKBR3, MCF7, and KatoIII target cells.

ACTR T Cells in Combination with Trastuzumab Mediate Robust Anti-Tumor Activity in the N87 Gastric Cancer Xenograft Model

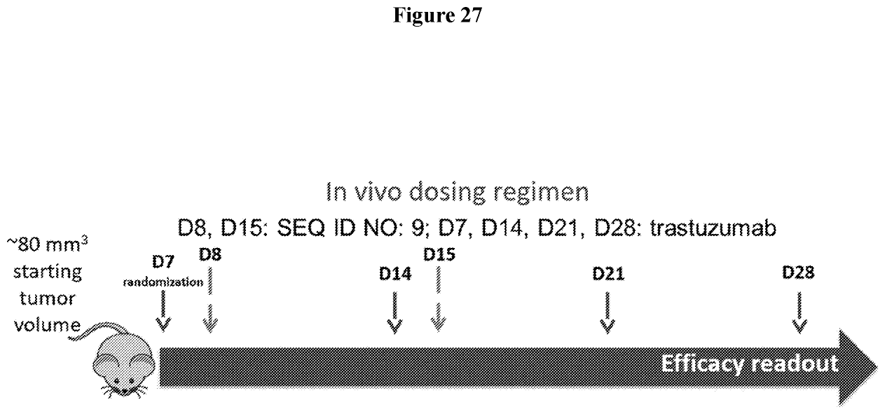

Experiments were performed using an in vivo dosing regimen for ACTR expressing T cells in combination with trastuzumab in female NSG (NOD.Cg-Prkd^{scid} IL-2rg^{tm1Wjl}/SzJ) mice with subcutaneous N87 (human gastric cancer cell line) tumors of approximately 80 mm³ starting volume. All in vivo procedures were carried out in accordance with IACUC approved protocols and standards, as described in Example 6. The dosing regimen is detailed in FIG. 27. Experimental groups in this study were vehicle (control; no treatment), trastuzumab alone, ACTR T cells alone, ACTR T cells+trastuzumab, and anti-HER2 CAR T cells.

The scFv in the anti-HER2 CAR was derived from trastuzumab and thus binds to the same epitope on HER2 as trastuzumab. Thus, the co-use of anti-HER2 CAR T cells and trastuzumab would not be expected to enhance anti-tumor activity in vivo, as they may compete against each other for binding to HER2⁺ tumor cells.

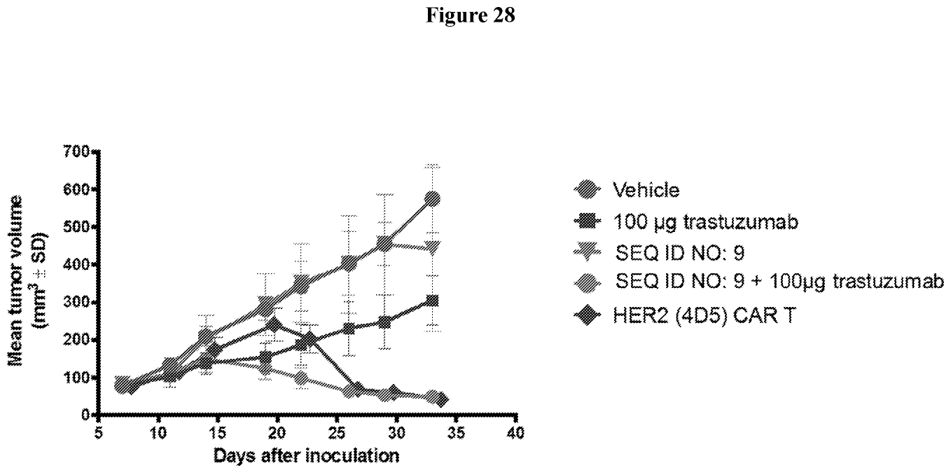

Groups that received trastuzumab were dosed intraperitoneally with 100 μg antibody once weekly for four weeks starting 7 days after tumor inoculation. Groups receiving ACTR T cells (1.5×10⁷ total T cells) or anti-HER2 CAR T cells (1×10⁷ total T cells) were dosed at day 8 and day 15 post-tumor inoculation. Control mice were administered vehicle alone on the same schedule for both antibody (vehicle is PBS) and T cells (vehicle is serum-free media). The mean tumor volume was measured throughout the experiment and plotted as a function of time (FIG. 28).

ACTR T cells, in combination with trastuzumab, showed robust anti-tumor activity relative to trastuzumab or ACTR T cells alone and showed faster anti-tumor kinetics than that observed with anti-HER2 CAR T cells.

HER2 Expression on Normal Cells Compared to Tumor Cell Lines

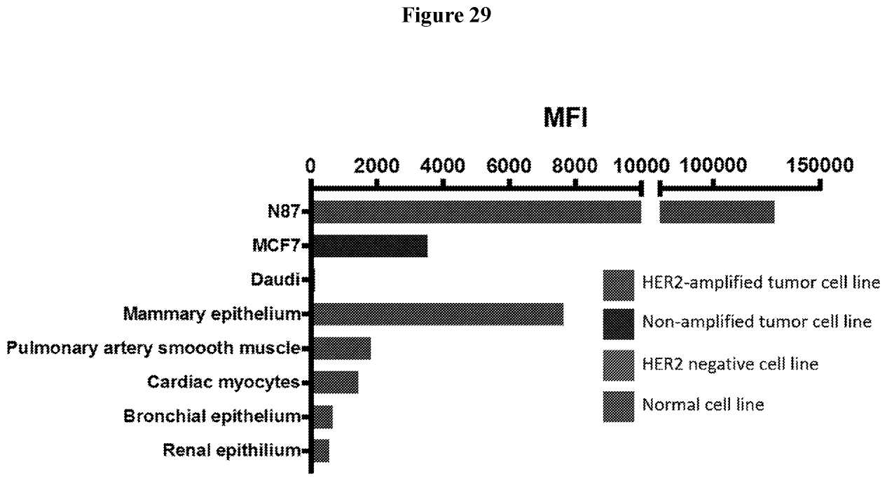

HER2 expression on HER2-amplified tumor cells (N87), non-HER-amplified tumor cells (MCF7), HER-2 negative cells (Daudi), and various normal cells (mammary epithelium, pulmonary artery smooth muscle, cardiac myocytes, bronchial epithelium, or renal epithelium) was measured by flow cytometry after staining with an anti-human HER2 antibody. The mean fluorescence intensity (MFI) was plotted for each cell type (FIG. 29). As expected, N87 cells showed high levels of HER2 expression, MCF7 cells showed low level of HER2 expression, and Daudi cells showed almost no HER2 expression. Among the normal cell lines, mammary epithelium cells showed a moderate level of HER2 expression while all others showed low levels of HER2 expression.

Differential In Vitro Activity of ACTR with Trastuzumab Compared to Anti-HER2 CAR T Cells on Normal Cells is Suggestive of a Favorable Therapeutic Index for ACTR+Trastuzumab

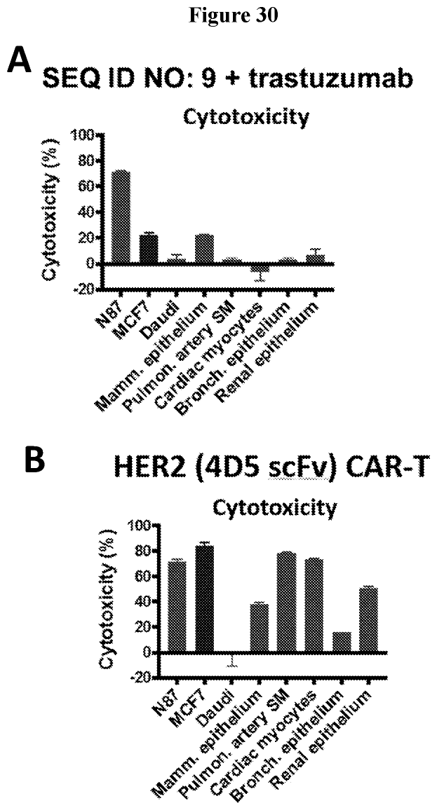

Cytotoxicity assays were carried out as described above with ACTR T cells or anti-HER2 CAR T cells and target cells at a 2:1 E:T for 48 hr; experiments with ACTR T cells also contained trastuzumab (0-5 μg/mL). Target cells evaluated in this experiment were N87 (HER2-amplified), MCF7 (HER2 non-amplified), Daudi (HER2 negative), and normal cell lines (mammary epithelium, pulmonary artery smooth muscle, cardiac myocytes, bronchial epithelium, and renal epithelium). ACTR T cells plus trastuzumab mediated robust cytotoxicity against HER2-amplified N87 cells and showed little or no activity against the non-amplified or negative target cell lines and the normal cell lines (data with ACTR in combination with 5 μg/mL trastuzumab plotted in FIG. 30, panel A). In contrast, anti-HER2 CAR T cells showed robust cytotoxicity against HER2-amplified N87 cells, HER2-non-amplified MCF7 cells, and several of the normal cell lines (FIG. 30, panel B). The results indicate that ACTR T cells in combination with trastuzumab have high tumor selectivity when compared to anti-HER2 CAR T cells.

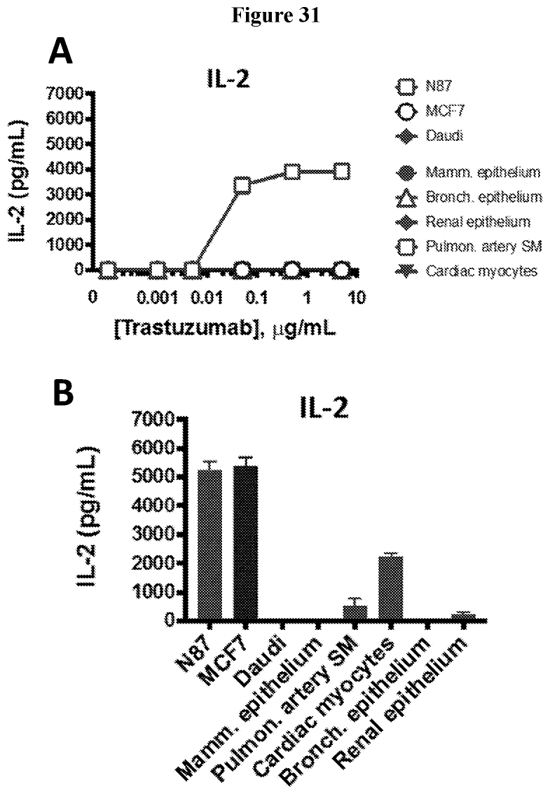

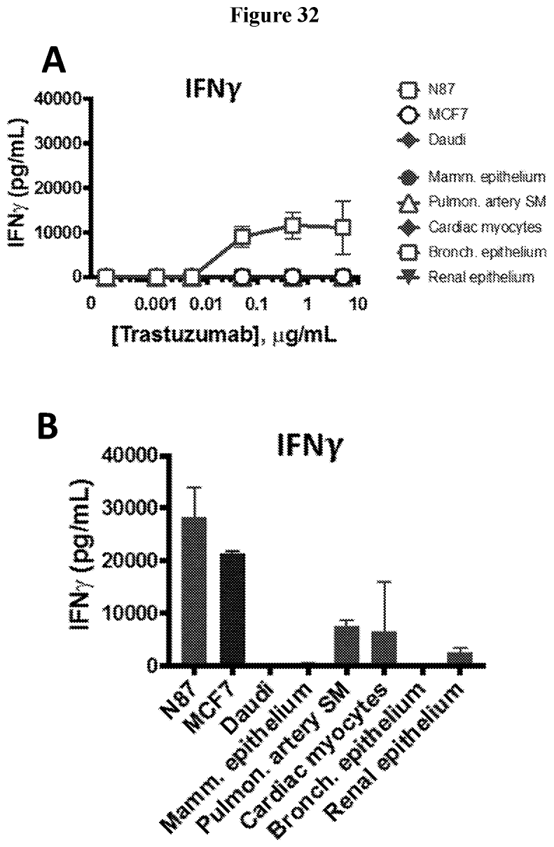

Supernatant was removed from the cytotoxicity reactions and analyzed for IL-2 and IFNγ release, as described above. ACTR T cells showed trastuzumab concentration-dependent IL-2 release in the presence of HER2-amplified N87 cells and little to no IL-2 release in the presence of the other target cells tested, including the normal cell lines (FIG. 31, panel A). In contrast, anti-HER2 CAR T cells showed robust IL-2 release in the presence of HER2-amplified N87 cells and HER2-non-amplified MCF7 cells and IL-2 release in the presence of normal pulmonary artery smooth muscle cells and cardiac myocytes (FIG. 31, panel B). ACTR T cells showed trastuzumab concentration-dependent IFNγ release in the presence of HER2-amplified N87 cells and little to no IFNγ release in the presence of the other target cells tested, including the normal cell lines (FIG. 32, panel A). In contrast, anti-HER2 CAR T cells showed robust IFNγ release in the presence of HER2-amplified N87 cells and HER2-non-amplified MCF7 cells and IFNγ release in the presence of normal pulmonary artery smooth muscle cells and cardiac myocytes (FIG. 32, panel B). These results demonstrate that ACTR-expressing T cells do not release cytokines in the presence of trastuzumab and normal primary cells (mammary epithelium, pulmonary artery smooth muscle, cardiac myocytes, bronchial epithelium, or renal epithelium), while cytokine release is observed with these normal cells in the presence of anti-HER2 CAR T cells.

Summary of Results

As shown above, when combined with trastuzumab, ACTR T cells had potent proliferation, cytokine production and tumor-directed cytotoxicity on HER2-amplified target cell lines, but reduced activity on the non-amplified MCF7 and KATOIII cell lines. Trastuzumab-based (4D5 scFv) HER2-targeting CAR-T cells demonstrated cytotoxicity and cytokine release on both HER2-amplified and non-amplified cell lines, suggesting a differential threshold of HER2 expression is required for activation of ACTR T cells compared to HER2 CAR-T cells. ACTR T cells had potent anti-tumor efficacy in a subcutaneous N87 gastric cancer xenograft model in NSG mice, and this activity was comparable to that of HER2 CAR-T cells. When incubated with normal cells in the presence of trastuzumab, ACTR T cells did not demonstrate significant cytotoxicity or cytokine release. In contrast, HER2 CAR-T cells released cytokines and mediated cytotoxicity against these normal cells. Taken together, these data support the efficacy of ACTR T cells+trastuzumab on HER2-amplified cells, and suggest a decreased risk of ‘on target/off tumor’ toxicity with this combination compared to trastuzumab-based HER2-targeting CAR T cells.

### Example 8: Effective Targeting of CD38-Positive Cancers with Daratumumab in Combination with T Cells Expressing ACTR Variant SEQ ID NO: 9

### CD38 Expression on the Surface of Multiple Myeloma and Lymphoma Cell Lines, Multiple Myeloma Plasma Cells, Immune Cells, and Red Blood Cells

CD38 expression was evaluated on the surface of cancer cell lines, patient-derived cells, and normal cells by flow cytometry.

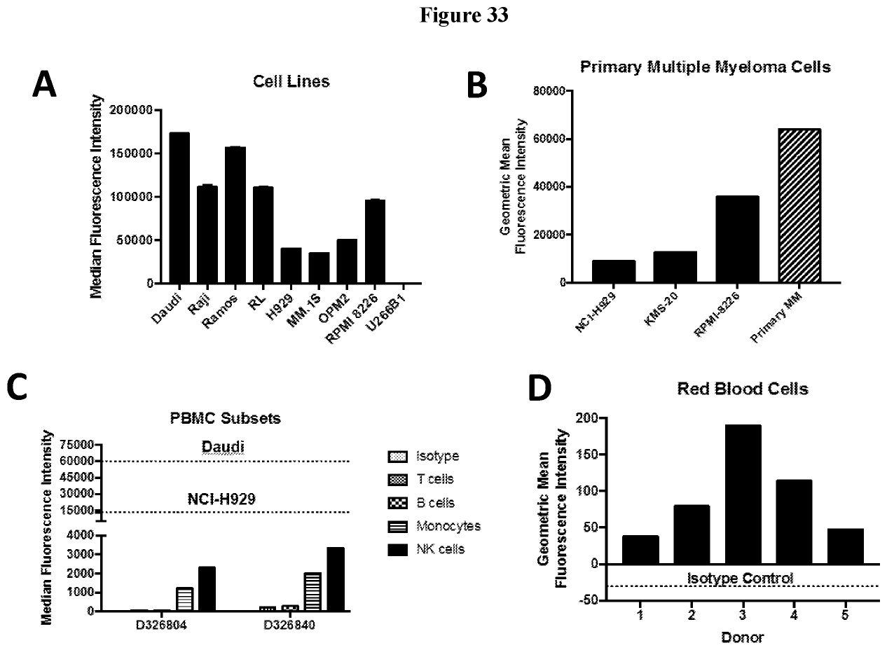

CD38 expression was evaluated on lymphoma cell lines Daudi, Raji, Ramos, and RL and multiple myeloma cell lines NCI-H929, MM.1S, OPM2, RPMI 8226, and U266B1. Cell lines were washed twice in PBS with BSA (staining buffer) followed by a 10-minute incubation with human Fc block. The cells were then incubated with 10 μg/mL daratumumab for 20 minutes at room temperature. This was followed by two washes in staining buffer and incubation of PE-conjugated goat anti-human IgG (Fab′)₂ secondary antibody for 30 min at 4 degrees C. Following two washes in staining buffer, stained cells were analyzed by flow cytometry. The geometric mean fluorescence intensity (gMFI) was plotted for each cell line (FIG. 33, panel A). All cell lines show CD38 expression to varying degrees except U266B1, which is negative for CD38 expression.

CD38 expression was also evaluated on NCI-H929, KMS-20, and RPMI-8226 multiple myeloma (MM) cell lines along with a primary MM patient-derived bone marrow mononuclear cell (BMMC) sample via flow cytometry. Patient derived BMMC and MM cell lines were washed once in PBS and then stained with a Live/Dead eFluor-780 dye for 30-minutes at 4 degrees C. Following viability staining, cells were washed once and incubated with human Fc block (50 μL) for 10 minutes at room temperature. The cells were then stained with 100 μL of an antibody cocktail consisting of AF488-conjugated anti-human CD38 antibody and Brilliant Violet 510-conjugated anti-human CD27 antibody. Following a 30-minute staining incubation, cells were washed twice with staining buffer and evaluated by flow cytometry. Flow cytometry gating on multiple myeloma cell lines and MM patient derived BMMC was performed after doublet exclusion and dead cell exclusion. CD38 expression was detected on all cells and the highest expression was observed with the patient-derived BMMC cells (FIG. 33, panel B).

CD38 expression was also evaluated on NCI-H929 multiple myeloma, Daudi lymphoma, and peripheral blood mononuclear cell (PBMC) subsets from two donors via flow cytometry. PBMCs were thawed and washed once with staining buffer. Cells were stained with 100 μL of an antibody cocktail consisting of AlexaFluor488-conjugated anti-human CD3, APC-conjugated anti-human CD19, PE-Cy7 conjugated anti-human CD14, Brilliant Violet 421-conjugated anti-human CD56, and PE-conjugated anti-human CD38 antibodies. Following, a 30 minute incubation at 4 degrees C., cells were washed twice with staining buffer and evaluated by flow cytometry. Following doublet exclusion, the gMFI of CD38 was calculated within the CD3+(T cell), CD19+(B cell), CD3− CD56+(natural killer (NK) cell), and CD3− CD14+(monocyte) populations. The CD38 expression level on PBMC subsets was compared to the CD38 expression (gMFI) level of Daudi and NCI-H929 cells. In both donors, NK cells demonstrated the highest CD38 expression of the immune cell subsets, but the level of CD38 expression was significantly lower than that observed on NCI-H929 multiple myeloma and Daudi lymphoma cells (FIG. 33, panel C).

CD38 expression was also evaluated on the surface of erythrocytes (red blood cells) from five different healthy donors. Fresh whole blood from five healthy donors was diluted 1:10000 in cell staining buffer containing sodium azide. Cells were washed once, followed by a 30-minute incubation with 100 μL of an antibody cocktail consisting of APC-conjugated anti-human CD235a, Brilliant Violet 605-conjugated anti-human CD45, and PE-conjugated anti-human CD38 antibodies. Following the incubation, cells were washed twice with cell staining buffer and evaluated by flow cytometry. Low CD38 expression (gMFI) was observed on the surface of CD235a+ erythrocytes (FIG. 33, panel D).

### CD38 Expression on the Surface of Activated ACTR T Cells

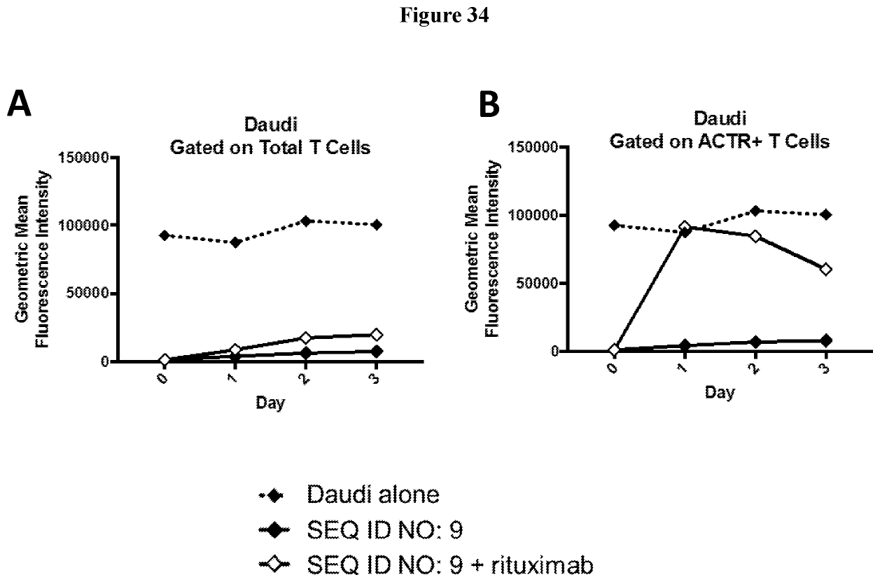

ACTR-expressing T cells (effector; E) and Daudi target cells (target; T) were incubated at a 1:1 effector-to-target ratio (30,000 target cells) in the presence of 1 μg/mL of CD20-targeting antibody rituximab. Reactions were incubated for 1, 2, or 3 days in a 37 degree C./5% CO₂ incubator, followed by staining for flow cytometry analysis. Briefly, cells were washed once with PBS followed by staining with fixable viability dye eFluor450. Cells were washed again with PBS, followed by incubation with 100 μL of an antibody cocktail containing AlexaFluor488-conjugated anti-human CD3, APC-conjugated anti-human CD16, and PE-conjugated anti-human CD38 antibodies. Following a twenty-minute incubation, cells were washed twice and data acquired on a flow cytometer. The gMFI of CD38 was determined on CD3+ total T cells and on CD3+CD16+ ACTR+ T cells, and Daudi target cells. The gMFI of CD38 was plotted as a function of time following stimulation (FIG. 34). The gMFI of CD38 increased as a function of time on total T cells (all CD3+ cells) after activation in the presence of rituximab and Daudi cells relative to cells incubated in the absence of rituximab, but the CD38 expression was significantly lower than that on Daudi target cells (FIG. 34, panel A). The gMFI of CD38 was significantly upregulated on ACTR T cells (CD3+CD16+ cells) in the presence of rituximab, with a peak expression 24 hours after stimulation that was similar to that observed on Daudi target cells (FIG. 34, panel B). These experiments demonstrate that CD38 is upregulated on ACTR T cells upon activation with a targeting-antibody and a target cell expressing the cognate antigen for the antibody.

### Production of Mock, ACTR, T Cells, and CD38-Targeting CAR-T Cells

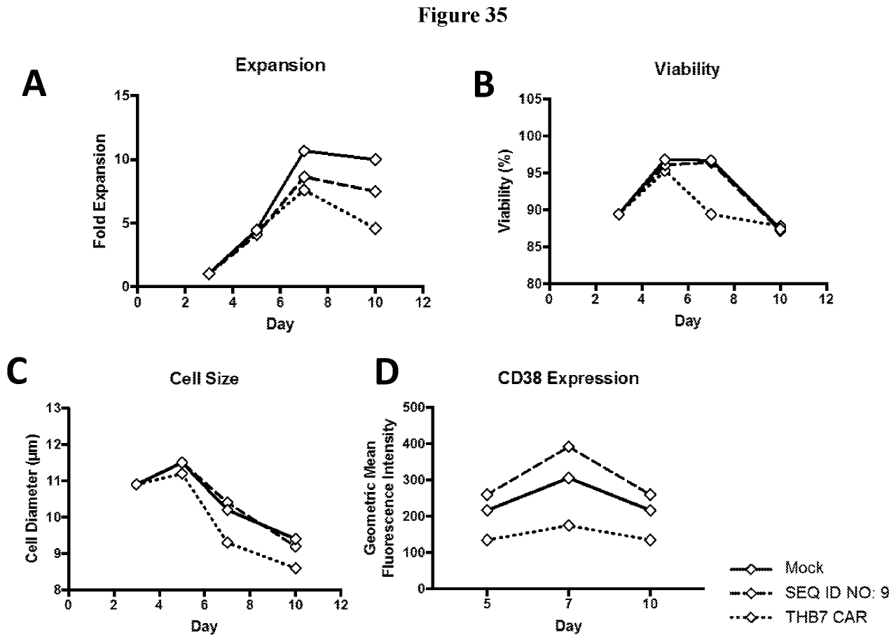

T cells were generated from PBMCs activated with anti-CD3 and anti-CD28 antibodies. Cells were transduced three days post-activation with a gamma-retrovirus that encoded ACTR or a CD38-targeting CAR sequence comprised of the THB7 single chain variable fragment linked to the 4-1BB costimulatory domain, and the T-cell receptor CD3zeta intracellular domain (THB7-41BB-CD3zeta) (Mihara et al. 2009. J Immunother. 32(7):737-43.); mock T cells were not transduced with virus. Fold expansion (FIG. 35, panel A), viability (FIG. 35, panel B), and cell size (FIG. 35, panel C) were monitored throughout the course of expansion using a Nucleocounter NC-200 cell counter. CD38 expression was also evaluated by flow cytometry on day 5, 7, and 10 of the expansion (FIG. 35, panel D). Briefly, cells were washed once in PBS with BSA (staining buffer) followed by a 20-minute incubation with 100 μL of an antibody cocktail containing APC-Cy7-conjugated anti-human CD3, Brilliant Violet 421-conjugated anti-human CD4, PE-conjugated anti-human CD8, APC-conjugated anti-human CD16, and FITC-conjugated anti-human CD38. Following two washes in staining buffer, detection was performed via flow cytometry. ACTR T cells show enhanced expansion (FIG. 35, panel A), viability (FIG. 35, panel B), cell diameter (FIG. 35, panel C), and CD38 expression (FIG. 35, panel D) relative to anti-CD38 CAR T cells.

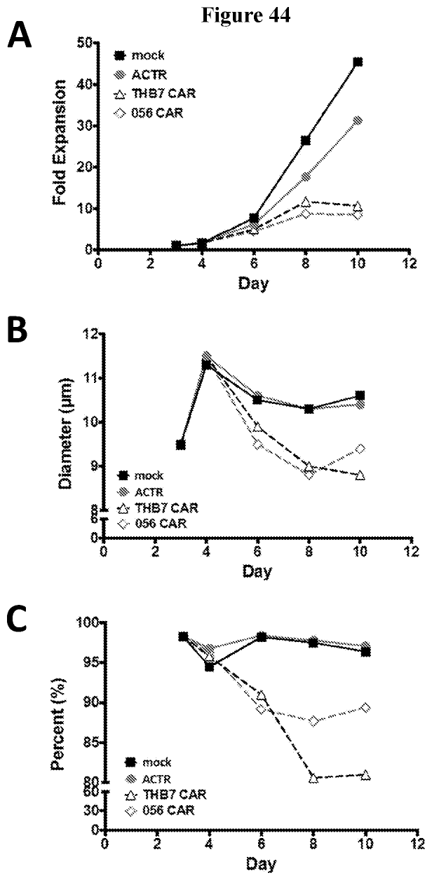

In a similar experiment, an additional anti-CD38 CAR was evaluated. The 056 CAR is comprised of the 056 single chain variable fragment linked to the 4-1BB costimulatory domain, and the T-cell receptor CD3zeta intracellular domain (056-41BB-CD3zeta) (Drent et al. 2016. Haematologica. 101(5):616-25). T cells were transduced with gamma-retrovirus that encoded ACTR, the THB7 CAR, or the 056 CAR; mock T cells were not transduced with virus. Fold expansion (FIG. 44, panel A), cell size (FIG. 44, panel B), and cell viability (FIG. 44, panel C) were monitored throughout the course of expansion using a Nucleocounter NC-200 cell counter. ACTR T cells showed enhanced expansion (FIG. 44, panel A), cell diameter (FIG. 44, panel B), and cell viability (FIG. 44, panel C) relative to T cells expressing both anti-CD38 CAR variants.

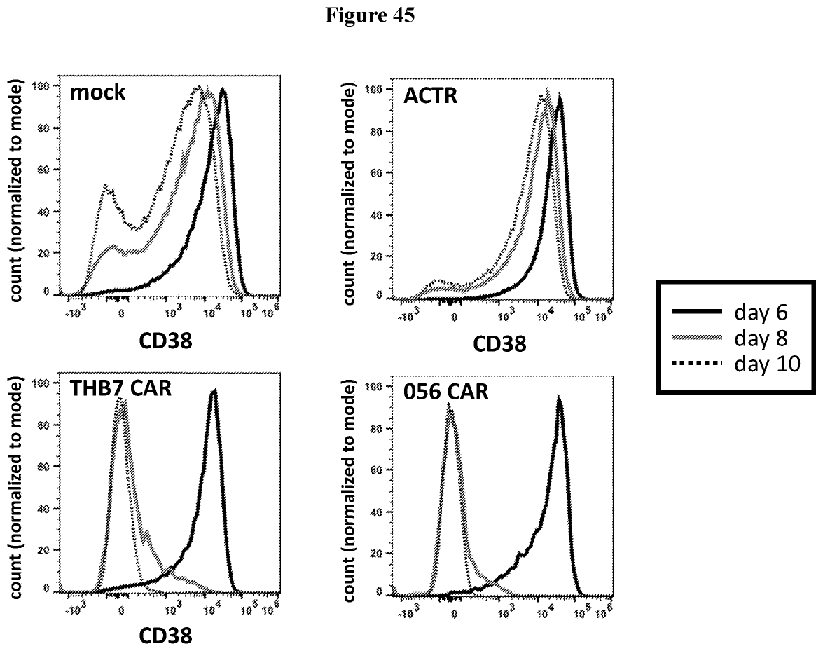

CD38 expression was also evaluated by flow cytometry on day 6, 8, and 10 of the expansion. Briefly, cells were washed once in PBS with BSA (staining buffer) followed by a 20-minute incubation with PE-conjugated anti-human CD38 antibody. Following two washes in staining buffer, detection was performed via flow cytometry. Histograms representing CD38 expression for each T cell expansion are shown in FIG. 45. Histograms for both mock and ACTR T cells demonstrate a shift towards lower CD38 expression over the course of expansion but maintain robust CD38 expression throughout the experiment. Histograms for both THB7 CAR and 056 CAR T cells show a marked decrease in CD38 expression over the course of the experiment, with little or no CD38 expression observed at day 8 and day 10 for both CAR variants, indicating CAR-mediated depletion of CD38-positive T cells and/or downregulation of CD38 expression in CAR+ cells.

These experiments demonstrate that anti-CD38 CAR T cell production is inhibited by CD38-target-mediated autolysis while ACTR T cell production is not.

### ACTR T Cells Demonstrate Enhanced Cytotoxicity and Cytokine Production in Comparison to Anti-CD38 CAR T Cells

The T cells generated in the experiments described above were evaluated in activity experiments with CD38-expressing target cells. ACTR- and CAR-expressing cells were normalized for matched transduction efficiency with mock T cells. For these experiments, mock T cells, ACTR T cells, THB7 CAR T cells, and 056 CAR T cells were incubated at different E:T ratios (1:4, 1:2, 1:1, 2:1, and 4:1) with CD38-expressing Daudi, NCI-H929, or RPMI-8226 target cells. Experiments with mock and ACTR T cells were carried out in the absence and presence of daratumumab (1 μg/mL). Reactions were incubated for 24 hr at 37 degrees C. in a 5% CO₂ incubator. Supernatant (100 μL) was removed for cytokine analysis.

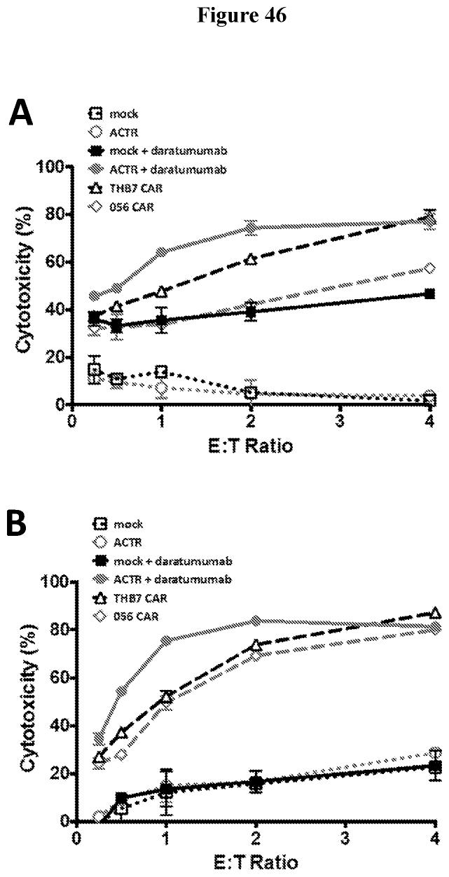

Cytotoxicity was evaluated by flow cytometry. Briefly, cells were washed once with PBS followed by staining with a fixable viability dye. Cells were washed again with PBS, followed by incubation with 100 μL AlexaFluor488-conjugated anti-human CD3 antibody. Following a twenty-minute incubation, cells were washed twice and data acquired on a flow cytometer. Live target cell counts were determined by gating on viability dye negative, CD3-cells. The percentage of live target cells was determined by dividing the live target cell count from a given sample by the live target cell count in the target cell alone wells. The percent cytotoxicity was determined by subtracting the percent live cells from 100. Percent cytotoxicity was plotted as a function of E:T ratio (FIG. 46).

An effector cell dose-dependent increase in cytotoxicity was observed for ACTR with daratumumab, THB7 CAR, and 056 CAR T cells cultured with Daudi (FIG. 46, panel A) and NCI-H929 (FIG. 46, panel B) cells. Mock T cells alone and ACTR T cells alone showed little or no cytotoxicity against either target cell line. Mock T cells in the presence of daratumumab showed some cytotoxicity against Daudi cells, indicating that daratumumab alone may have a cytotoxic effect on these target cells (FIG. 46, panel A). ACTR T cells in the presence of daratumumab demonstrated superior cytotoxicity to both the THB7 and 056 CAR T cells at lower E:T ratios with both Daudi and NCI-H929 target cells (FIG. 46, panels A and B, respectively).

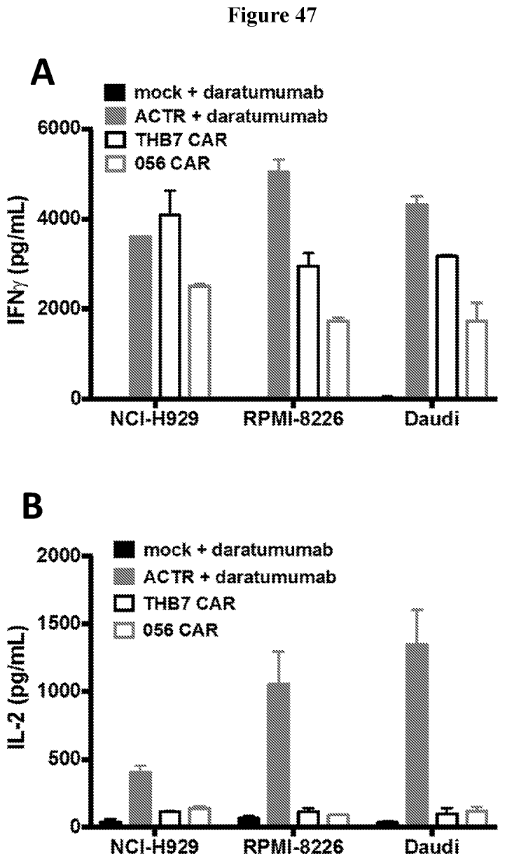

Supernatants from these experiments were analyzed for IFNγ and IL-2 using the Cisbio homogenous time resolved fluorescence (HTRF) assay. Briefly, the cytokine standards and conjugates were prepared according to the manufacturer protocol. In a low volume 384-well plate, a 10 μL volume of conjugate and 10 μL volume of cell supernatant were co-incubated for 2 hours (IL-2) or 24 hours (IFNγ). Plates were read on an EnVision Multi-label plate reader. The concentration of IFNγ or IL-2 measured in the cell supernatant from reactions carried out at a 1:1 E:T ratio is plotted as a function of target cell (FIG. 47).

Robust IFNγ production was observed for ACTR T cells in the presence of daratumumab, THB7 CAR T cells, and 056 CAR T cells in the presence of NCI-H929, RPMI-8226, and Daudi target cells (FIG. 47, panel A). IFNγ production with mock T cells in the presence of daratumumab was very low or below the limits of quantitation for the assay. Robust IL-2 production was observed for ACTR T cells in the presence of daratumumab in the presence of NCI-H929, RPMI-8226, and Daudi target cells (FIG. 47, panel B). Low levels of IL-2 production were observed for THB7 CAR, 056 CAR, and mock (in the presence of daratumumab) T cells in the presence of NCI-H929, RPMI-8226, and Daudi target cells (FIG. 47, panel B).

These experiments demonstrate that ACTR T cells in the presence of daratumumab have superior cytotoxicity and cytokine production relative to anti-CD38 CAR T cells.

### ACTR T Cell Cytotoxicity Mediated Against NCI-H929, MM.1S, RPMI-8226, and Daudi Cells is Dose-Dependent in the Presence of Daratumumab and ACTR-Specific

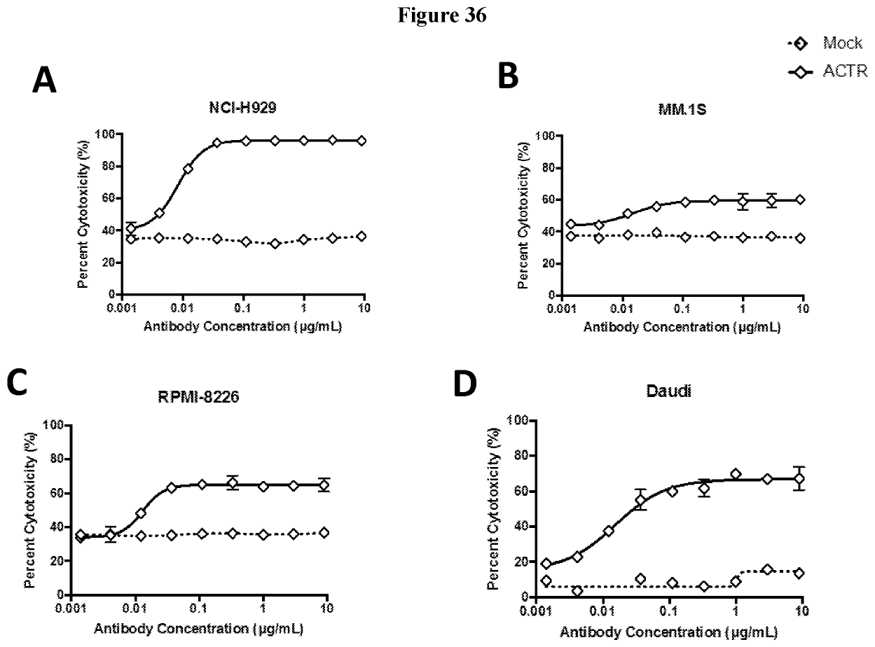

ACTR or mock T cells (effector; E) and target cells (target; T) were incubated at a 2:1 E:T ratio in the presence of increasing concentrations of the CD38-targeting antibody, daratumumab. For these experiments, NCI-H929, MM.1S, RPMI-8226, and Daudi target cells were used. Reactions were incubated in a CO₂ (5%) incubator at 37 degrees C. for 24 hours, followed by flow cytometry staining. Briefly, cells were washed once with PBS followed by staining with a fixable viability dye. Cells were washed again with PBS, followed by incubation with 100 μL of antibody cocktail containing AlexaFluor488-conjugated anti-human CD3 and AlexaFluor 647-conjugated anti-human CD16 antibodies. Following a thirty-minute incubation, cells were washed twice and data acquired on a flow cytometer. Live target cell counts were determined by gating on viability dye negative, CD3-CD16− cells. The percentage of live target cells was determined by dividing the live target cell count from a given sample by the live target cell count in the target cell alone wells. The percent cytotoxicity was determined by subtracting the percent live cells from 100. Percent cytotoxicity was plotted as a function of antibody concentration (FIG. 36).

An antibody dose dependent increase in cytotoxicity was observed for ACTR T cells cultured with NCI-H929 (FIG. 36, panel A), MM.1S (FIG. 36, panel B), RPMI-8226 (FIG. 36, panel C), and Daudi (FIG. 36, panel D) target cells in the presence of daratumumab. An increase in cytotoxicity was not observed when mock T cells were cultured in the presence of increasing concentrations of daratumumab.

### ACTR T Cell Cytokine Release in the Presence of NCI-H929, MM.1S, RPMI-8226, and Daudi Cells and Daratumumab is Antibody-Dose-Dependent

ACTR or mock T cells (effector; E) and target cells (target; T) were incubated at a 1:1 E:T ratio in the presence of increasing concentrations of the CD38-targeting antibody daratumumab. Cell supernatants were collected following a 24-hour incubation in a 37 degrees C./5% CO₂ incubator. Supernatants were analyzed for IFNγ and IL-2 using the Cisbio homogenous time resolved fluorescence (HTRF) assay. Briefly, the cytokine standards and conjugates were prepared according to the manufacturer protocol. In a low volume 384-well plate, a 10 μL volume of conjugate and 10 μL volume of cell supernatant were co-incubated for 2 hours (IL-2) or 24 hours (IFNγ). Plates were read on an EnVision Multi-label plate reader. The concentration of IFNgamma or IL-2 measured in the cell supernatant is plotted as a function of daratumumab concentration.

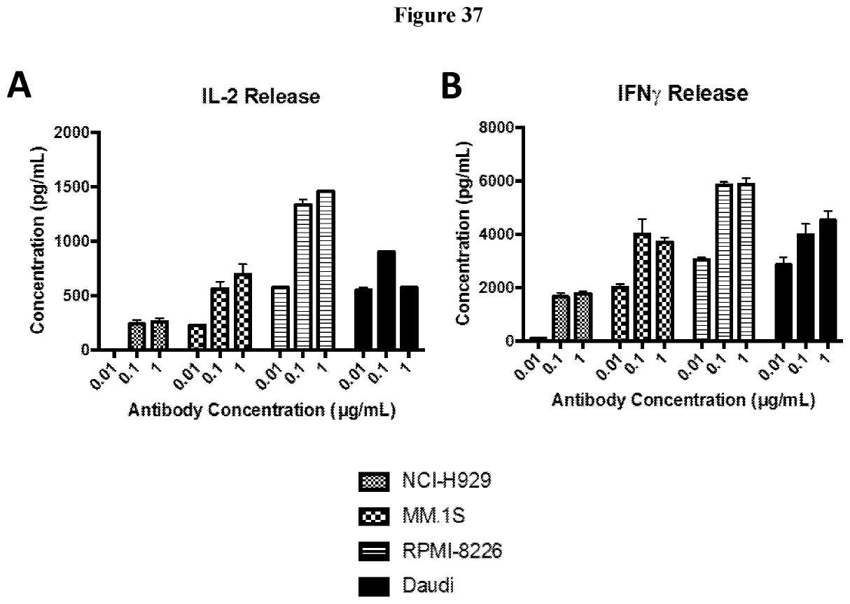

Robust ACTR T cell (FIG. 37, panel A) IL-2 and (FIG. 37, panel B) IFNγ production was observed in the presence of daratumumab-opsonized NCI-H929, MM. 1S, RPMI-8226, and Daudi target cells. Cytokine production with mock T cells (not plotted) was below the linear range of the standard curve.

### ACTR T Cell Specific Proliferation in the Presence of Daratumumab-Opsonized NCI-H929, MM.1S, RPMI-8226, and Daudi Target Cells

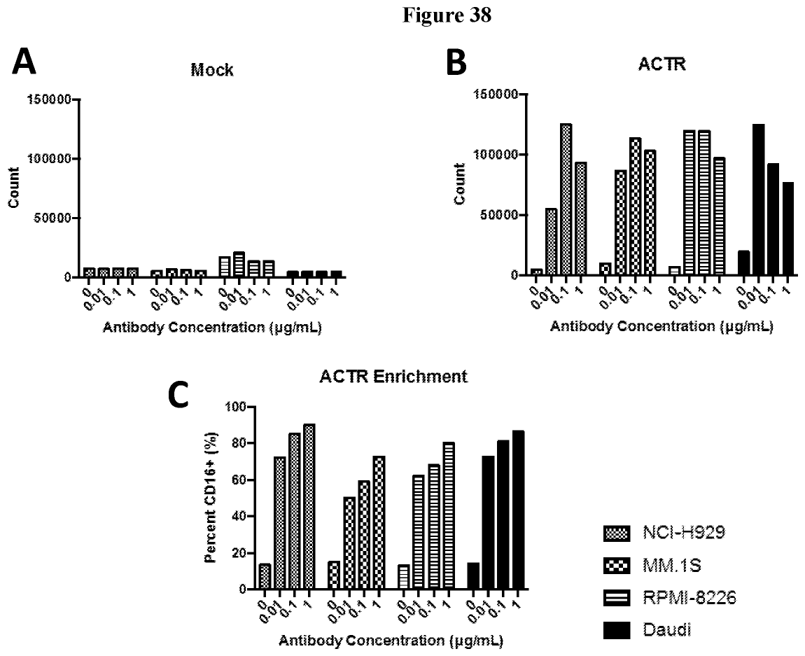

T cells were transduced with gamma retrovirus encoding ACTR; flow cytometry experiments demonstrated that 24-32% of these cells were positive for ACTR. ACTR or mock T cells (effector; E) and target cells (target; T) were incubated at a 1:1 effector-to-target ratio in the presence of increasing concentrations of the CD38-targeting antibody daratumumab. Reactions were incubated for 7 days in a 37 degrees C./5% CO₂ incubator, followed by flow cytometry staining. Briefly, cells were washed once with PBS followed by staining with fixable viability dye eFluor450. Cells were washed again with PBS, followed by incubation with 100 μL of an antibody cocktail containing AlexaFluor488-conjugated anti-human CD3 and AlexaFluor647-conjugated anti-human CD16 antibodies. Following a thirty-minute incubation, cells were washed twice and data acquired on a flow cytometer. Live T cell counts were determined by gating on viability dye negative, CD3+ cells. In FIG. 38, panels A and B, total T cell count is plotted as a function of daratumumab antibody concentration. Mock T cells did not proliferate in the absence or presence of daratumumab and NCI-H929, MM.1S, RPMI-8226, and Daudi target cells (FIG. 38, panel A). ACTR T cells demonstrated robust proliferation in the presence of NCI-H929, MM.1S, RPMI-8226, and Daudi target cells in the presence, but not in the absence of daratumumab (FIG. 38, panel B). In FIG. 38, panel C, the percentage of CD16+ cells was calculated within the total CD3+ T cell gate, and plotted as a function of daratumumab antibody concentration. The percentage of ACTR+ T cells increased in an antibody dose dependent manner demonstrating that ACTR+ T cells are preferentially enriched over untransduced cells during proliferation.

### ACTR T Cell Cytotoxicity and Cytokine Release is Minimal Against Autologous PBMC Subsets but Specific to CD38 Expressing Target Cell Lines.

ACTR T cells were evaluated for potential targeting of PBMCs and PBMC subsets in the presence of CD38-targeting daratumumab in a co-culture assay that contained ACTR T cells, autologous PBMCs, and a CD38-expressing multiple myeloma target cell line. As described above, low expression of CD38 is observed on some PBMC subsets (FIG. 33, panel C).

ACTR or mock T cells were incubated in the presence of autologous PBMCs at a 1:1 E:T ratio (100,000 cells each), with or without RPMI-8226 multiple myeloma target cells (25,000 cells) at a 4:1 E:T ratio. Reactions were incubated at 37 degrees C./5% CO₂ for 24 hours in the presence or absence of daratumumab. After a 24 hours, half of the cell supernatant was collected for cytokine analysis and the cells were harvested for flow cytometry analysis.

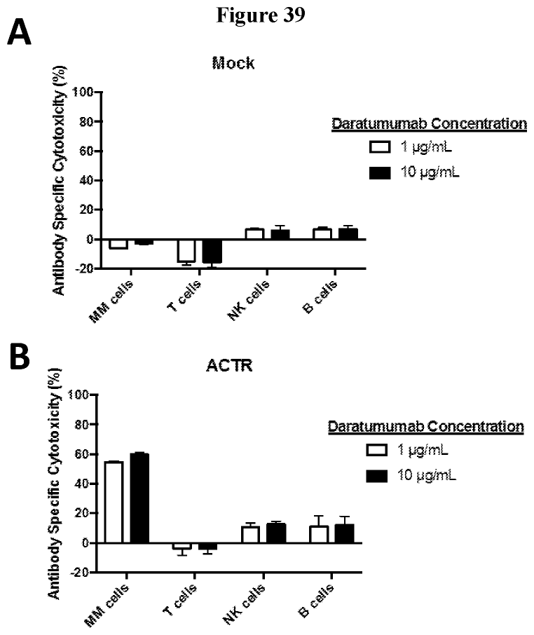

Briefly, cells were washed once with PBS followed by staining with fixable viability dye eFluor780. Following viability staining, cells were washed once with PBS and incubated for 30-minutes with 100 μL of an antibody cocktail consisting of AlexaFluor488-conjugated anti-human CD3, APC-conjugated anti-human CD19, PE-Cy7 conjugated anti-human CD14, Brilliant Violet 421-conjugated anti-human CD56, and Brilliant Violet 510-conjugated anti-human CD138. Cells were washed twice with staining buffer and evaluated by flow cytometry. Following doublet exclusion and dead cell exclusion, CD3+ T cell, CD19+ B cell, CD3− CD56+ natural killer cell, CD3− CD14+ monocyte, and CD3− CD138+ MM target cell counts were determined. The percentage of live target cells was calculated for each specified cell subset by dividing the live target cell count in a given sample well by the live target cell count in the absence of antibody. The antibody specific cytotoxicity was determined by subtracting the percent live cells from 100. Antibody specific cytotoxicity (%) is plotted as a function of antibody concentration for the various cell subsets for reactions with mock T cells (FIG. 39, panel A) and with ACTR T cells (FIG. 39, panel B) in the presence of 1 or 10 μg/mL daratumumab. Data is representative of three donors. Little or no cytotoxicity was observed with mock T cells; ACTR T cells showed robust cytotoxicity against RPMI-8226 cells (MM cells) but little to no cytototoxicity against autologous PBMC subsets.

Cytokines from supernatants were analyzed as described above. Briefly, half of the cell supernatant (100 μL) was collected for IFNγ and IL-2 cytokine analysis using a Cisbio homogenous time resolved fluorescence (HTRF) assay. The cytokine standards and conjugates were prepared according to the manufacturer's protocol. In a low volume 384-well plate, a 10 μL volume of conjugate and 10 μL volume of cell supernatant were co-incubated for 2 hours (IL-2) or 24 hours (IFNγ). Plates were read on an EnVision Multi-label plate reader. The concentration of cytokine measured in the cell supernatant is plotted as a function of daratumumab antibody concentration for reactions with ACTR T cells and PBMCs, with or without RPMI-8226 cells (FIG. 40). An increase in IFNγ (FIG. 40, panel A) and and IL-2 (FIG. 40, panel B) production was observed when ACTR T cells were cultured with daratumumab in the presence, but not in the absence, of RPMI-8226 multiple myeloma cells.

ACTR T Cells in Combination with Daratumumab do not Mediate Hemolysis.

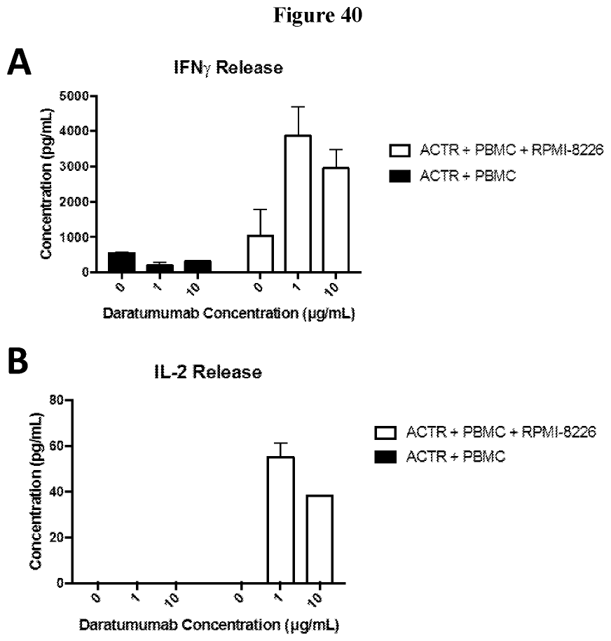

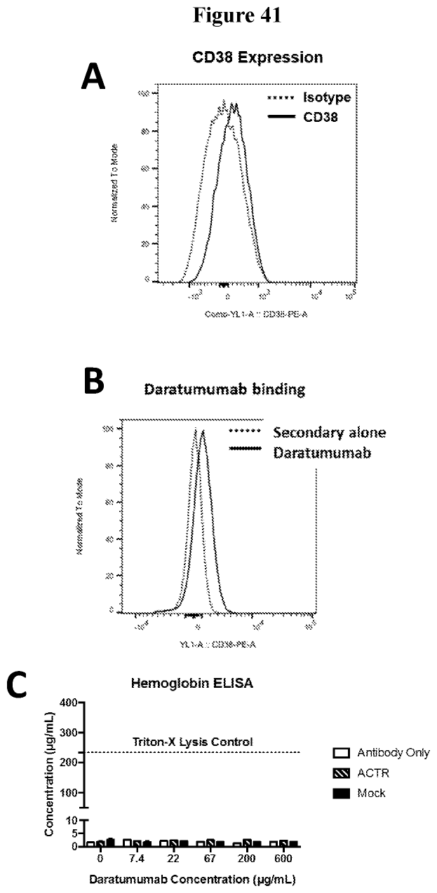

CD38 expression was evaluated on red blood cells by flow cytometry. Fresh whole blood from five healthy donors was diluted 1:10000 in cell staining buffer containing sodium azide (cell staining buffer). Cells were washed once with cell staining buffer, followed by a 30-minute incubation with 100 μL of an antibody cocktail consisting of APC-conjugated anti-human CD235a, Brilliant Violet 605-conjugated anti-human CD45, and PE-conjugated anti-human CD38 or isotype control antibodies. Following the incubation, cells were washed twice with cell staining buffer and evaluated by flow cytometry. CD38 expression was evaluated on the surface of CD235a+ erythrocytes and is plotted in FIG. 41, panel A. The resulting histogram of erythrocytes stained with an anti-CD38 antibody showed a slight shift relative to staining with isotype control, indicating a small amount of CD38 expression on erythrocytes.

Binding of daratumumab to red blood cells was also evaluated by flow cytometry. Fresh whole blood from five healthy donors was diluted 1:1000 in cell staining buffer. Diluted whole blood (100 μL) was plated in a 96 well round bottom plate and pelleted by centrifugation at 200×g for 5 minutes. Cells were incubated for 30 minutes at 37 degrees C./5% CO₂ in cell staining buffer containing daratumumab prepared at a final concentration of 8 μg/mL. The cells were then washed twice with cell staining buffer and incubated with a PE-conjugated goat anti-human IgG (Fab′)₂ secondary antibody for 20 minutes at room temperature. Following two washes in staining buffer, PE detection was performed via flow cytometry. Maximum daratumumab binding is displayed for one of five donors tested, and compared to secondary antibody alone (FIG. 41, panel B). A small shift in the resulting histogram was observed in the presence of daratumumab relative to the secondary antibody alone, indicating some daratumumab binding to red blood cells.

ACTR T cell mediated hemolysis was evaluated using an ACTR and red blood cell coculture assay. ACTR T cells were incubated in the presence of red blood cells from five healthy donors to achieve an approximate 1:200 ACTR+ T cell: red blood cell ratio. Reactions with untransduced mock T cells and daratumumab and with daratumumab alone in the absence of T cells were used as a controls in this experiment. Reactions were incubated in the presence of daratumumab at 600 μg/mL, 200 μg/mL, 66.7 μg/mL, 22.2 μg/mL, 7.4 μg/mL or 0 μg/mL for 24 hours in a 37 degree C./5% CO₂ incubator. Following the 24 hour co-culture, viable red blood cells were pelleted by centrifugation at 500×g for 10 minutes. Supernatant (60 μL) was collected for analysis in a hemoglobin ELISA, where hemoglobin measured in the cell supernatant is indicative of red blood cell lysis. Briefly, wash buffer, diluent solution, enzyme-antibody conjugate, and the hemoglobin standard were prepared according to the manufacturer's instructions. Sample and standard (100 μL) were added to the ELISA plate and incubated for 20 minutes at room temperature. The plate was washed four times with 300 μL of wash buffer, followed by a 20 minute incubation with 100 μL enzyme-antibody conjugate. The plate was again washed four times with 300 μL of wash buffer, followed by a 10 minute incubation with 100 μL TMB chromogen substrate solution. Stop solution (100 μL) was added to each well and the absorbance at 450 nm was determined using a SpectraMax I3X. The hemoglobin concentration measured in each sample well was compared to a 2% Triton-X addition that leads to total lysis (control dotted line). Hemoglobin concentration was plotted as a function of daratumumab concentration (FIG. 41, panel C). Little to no hemoglobin was detected in the supernatants from reactions and there was no difference among reactions with ACTR T cells, mock T cells, or daratumumab alone.

### Example 9: IL-2 Production from T Cells Expressing ACTR Variants with a CD28 Costimulatory Domain

Gamma-retroviruses were generated that encoded ACTR variants with SEQ ID NOs: 2, 9, 13, 19, 20, 21, 22, and 27. These viruses were used to infect primary human T-cells from two different donors, generating cells that expressed these ACTR variants on the surface of infected cells. These cells were subsequently used in IL2 release assays with Her2-positive HCC1954 or SKBR3 target cells and Her2-targeting trastuzumab.

T-cells (effector; E) and HCC1954 target cells (target; T) were incubated at a 1:1 effector-to-target ratio (120,000 effector cells; 120,000 target cells) in the presence of 1 μg/mL trastuzumab in a 200-μL reaction volume in RPMI 1640 media supplemented with 10% fetal bovine serum. T-cells (effector; E) and SKBR3 target cells (target; T) were incubated at a 1:1 effector-to-target ratio (30,000 effector cells; 30,000 target cells) in the presence of 1 μg/mL trastuzumab in a 100-μL reaction volume in RPMI 1640 media supplemented with 10% fetal bovine serum. Reactions were incubated in a CO₂ (5%) incubator at 37 degrees C. for 24 hours.

Supernatants were analyzed for IL-2 using a homogenous time resolved fluorescence (HTRF) assay (Cisbio). Briefly, the cytokine standards and conjugates were prepared according to the manufacturer's protocol. In a low volume 384-well plate, a 10-μL volume of conjugate and 10-μL volume of cell supernatant were co-incubated for 24 hours. The fluorescence signal was measured using an EnVision Multi-label plate reader and data was analyzed according to the manufacturer's recommendations.

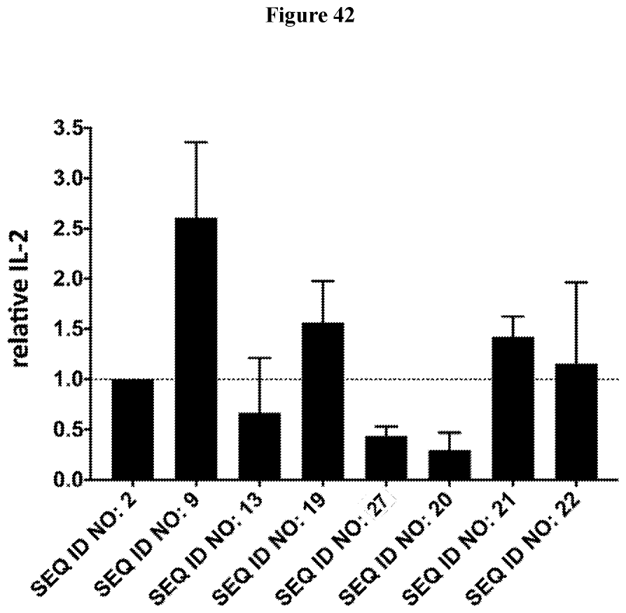

For each target/donor pair, the amount of IL-2 released was normalized to that released with ACTR variant SEQ ID NO: 2. The average relative IL-2 is plotted as a function of ACTR variant in FIG. 42. T cells expressing all ACTR variants tested showed production of IL-2, an indicator of T cell activity. ACTR variant SEQ ID NO: 9 showed superior IL-2 production relative to the other ACTR variants.

### Example 10: Cytokine Production with ACTR Variants SEQ ID NO: 9 and SEQ ID NO: 26

The ability of ACTR variants SEQ ID NOs 9 and 26 to generate cytokines was evaluated in the presence of a number of different antibody-target pairs. For these experiments, ACTR T cells expressing SEQ ID NO: 9 or SEQ ID NO: 26 were incubated at a 1:1 E:T ratio with target cells and varying concentrations of targeting antibodies at 37 degrees C. in a 5% CO₂ incubator for 24 hr. Mock T cells that did not express ACTR were included as a control. Supernatants were removed and analyzed for cytokines IL-2 and IFN-γ using the Meso Scale Discovery V-Plex Human IFN-γ and the V-Plex Human IL-2 kit, as described in Example 6. For experiments with HER2-expressing BT20 and SKBR3 target cells, trastuzumab was titrated from 0-0.5 μg/mL; for experiments with CD20-expressing Daudi, Raji, and RL target cells, rituximab was titrated from 0-10 μg/mL; for experiments with B7H3-expressing BT474 target cells, anti-B7H3 antibody hu8H9-6m (Ahmed et al. 2015. J. Biol. Chem., 290, pp. 30018-30029). Rituximab and trastuzumab were obtained from commercial sources. The anti-B7H3 antibody hu8H9-6m was generated by transfecting plasmids encoding the heavy and light chains of the antibody into Freestyle 293F cells (Thermo Scientific) and purifying the antibody from the cell culture supernatant using Protein A affinity.

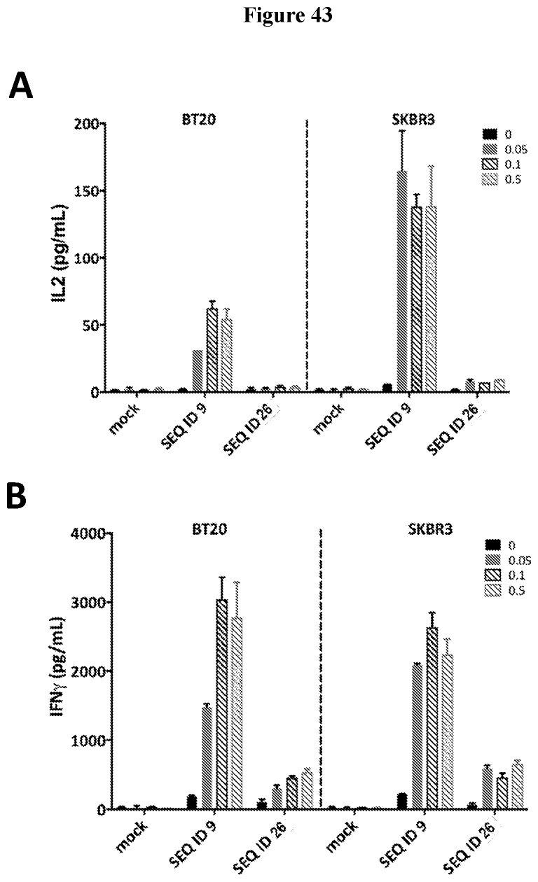

For experiments with HER2-expressing cell lines, IL-2 (FIG. 43, panel A) and IFN-γ (FIG. 43, panel B) values were plotted as a function of the T cells tested for different trastuzumab concentrations and target cell lines. Both ACTR variant SEQ ID NO: 9 and ACTR variant SEQ ID NO: 26 produced IL-2 and IFN-γ in an antibody-dependent manner. Both IL-2 and IFN-γ release were higher with ACTR variant SEQ ID NO: 9 than with ACTR variant SEQ ID NO: 26 with trastuzumab and HER2-expressing target cells. Similar results were obtained with rituximab and CD20-expressing target cells and anti-B7H3 antibody and B7H3-expressing target cells. The relative IL-2 and IFN-γ produced for each target-antibody pair at the highest antibody tested with ACTR variant SEQ ID NO: 9 and ACTR variant SEQ ID NO: 26 are shown in Table 8. Similar experiments were carried out with additional target-antibody pairs and also showed increased cytokine release with ACTR variant SEQ ID NO: 9 relative ACTR variant SEQ ID NO: 26.

TABLE 8
Relative cytokine release with ACTR variant SEQ ID NOs: 9 and 26

| | | | antibody | relative IL-2 | relative IFN-γ |
|---|---|---|---|---|---|
| target | cell line | | concentration | (SEQ ID 9/ | (SEQ ID 9/ |
| cell line | type | target | (μg/mL) | SEQ ID 26) | SEQ ID 26) |
| Daudi | hematalogic | CD20 | 10 | 34 | 2 |
| Raji | hematalogic | CD20 | 10 | 16 | 1 |
| RL | hematalogic | CD20 | 10 | 41 | 4 |
| BT20 | solid tumor | HER2 | 0.5 | 17 | 5 |
| SKBR3 | solid tumor | HER2 | 0.5 | 16 | 3 |
| BT474 | solid tumor | B7H3 | 5 | 123 | 4 |

Results of this study indicate that T cells expressing ACTR constructs containing a CD28 co-stimulatory domain (and optionally a CD28 transmembrane domain and/or a CD28 hinge domain) showed certain superior effects such as the cytokine release profile, indicating that such ACTR constructs may have certain superior therapeutic aspects (e.g., for treatment of cancers (hematopoietic or non-hematopoietic) lacking expression of co-stimulatory molecules).

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one of skill in the art can easily ascertain the essential characteristics of the present disclosure, and without departing from the spirit and scope thereof, can make various changes and modifications of the disclosure to adapt it to various usages and conditions. Thus, other embodiments are also within the claims.

1. An antibody-coupled T cell receptor (ACTR) polypeptide, comprising:
(i) a CD16A extracellular domain,
(ii) a transmembrane domain,
(iii) one or more co-stimulatory signaling domains, at least one of which is a CD28 co-stimulatory signaling domain, and
(iv) a CD3ζ cytoplasmic signaling domain;
wherein if the transmembrane domain (ii) is a CD8 transmembrane domain, the ACTR polypeptide is either free of a hinge domain from any non-CD16A receptor, or comprises more than one co-stimulatory signaling domains.

2. The ACTR polypeptide of claim 1, which further comprises a hinge domain, wherein the hinge domain is 1 to 60 amino acid residues in length.

3. The ACTR polypeptide of claim 2, wherein the hinge domain is 1 to 30 amino acid residues in length.

4. The ACTR polypeptide of claim 2, wherein the hinge domain is 31 to 60 amino acid residues in length.

5. The ACTR polypeptide of claim 2, wherein the hinge domain is a CD16A hinge domain, a non-CD16A receptor hinge domain, or a combination thereof.

6. The ACTR polypeptide of claim 2, wherein the hinge domain comprises a CD28 hinge domain.

7. The ACTR polypeptide of claim 1, wherein the transmembrane domain (ii) is a CD28 transmembrane domain.

8. The ACTR polypeptide of claim 1, which comprises (i) the CD28 co-stimulatory domain; and (ii) a CD28 transmembrane domain, a CD28 hinge domain, or a combination thereof.

9. The ACTR polypeptide of claim 8, which comprises the amino acid sequence of SEQ ID NO: 9, SEQ ID NO: 19, SEQ ID NO: 20, SEQ ID NO: 21, SEQ ID NO: 22, or SEQ ID NO: 27.

10. The ACTR polypeptide of claim 1, which comprises two co-stimulatory signaling domains, one being the CD28 co-stimulatory signaling domain and the other being a 4-1BB co-stimulatory signaling domain or an OX40 co-stimulatory signaling domain.

11. The ACTR polypeptide of claim 10, wherein the transmembrane domain (ii) is a CD8 transmembrane domain.

12. The ACTR polypeptide of claim 11, which further comprises a CD8 hinge domain.

13. The ACTR polypeptide of claim 1, which is free of a hinge domain from any non-CD16A receptor.

14. An antibody-coupled T cell receptor (ACTR) polypeptide, comprising:
(i) a CD16A extracellular domain,
(ii) a transmembrane domain, and
(iii) a CD3ζ cytoplasmic signaling domain;
wherein the ACTR polypeptide is free of a hinge domain from any non-CD16A receptor.

15-26. (canceled)

27. A nucleic acid, comprising a first nucleotide sequence encoding a first polypeptide that is an ACTR polypeptide of claim 1.

28-42. (canceled)

43. An immune cell expressing a first polypeptide, which is an antibody-coupled T cell receptor (ACTR) polypeptide of claim 1.

44. The immune cell of claim 43, wherein the immune cell is a T cell or a natural killer (NK) cell.

45. The immune cell of claim 43, which further expresses a second polypeptide, which comprises co-stimulatory domain or a ligand of a co-stimulatory receptor.

46. The immune cell of claim 45, wherein the second polypeptide comprises 4-1BBL, CD80, CD86, OX40L, ICOSL, CD70, or a combination thereof.

47. (canceled)

48. A method for enhancing antibody-dependent cell-mediated cytotoxicity in a subject, the method comprising administering to a subject in need thereof (i) an effective amount of an immune cell of claim 43, and (ii) an effective amount of a therapeutic antibody.

49-50. (canceled)

51. The method of claim 48, wherein the immune cell is an autologous T cell isolated from the subject, or an allogeneic T cell.

52-54. (canceled)

55. The method of claim 48, wherein the subject is a human patient having or suspected of having cancer.

56. A method for preparing immune cells expressing an antibody-coupled T cell receptor (ACTR), the method comprising introducing a nucleic acid of claim 27 into a population of immune cells.

57-58. (canceled)

59. A genetically engineered immune cell, expressing:
(i) a first polypeptide which is an antibody-coupled T cell receptor (ACTR), wherein the ACTR comprises a CD28 cytoplasmic signaling domain; and
(ii) a second polypeptide that elicits a co-stimulatory signal.

60-62. (canceled)

63. A method for treating a solid tumor, comprising:
(i) administering to a subject in need thereof an effective amount of one or more lymphodepleting agents;
(ii) administering to the subject an anti-CD20 antibody after (i); and
(iii) administering to the subject immune cells expressing an antibody-coupled T cell receptor (ACTR) after (ii), wherein the ACTR comprises the amino acid sequence of SEQ ID NO:9.

64-77. (canceled)

78. A method for inducing cytotoxicity in a subject, comprising administering to a subject in need thereof
(i) an antibody specific to an antigen expressed on the surface of activated T cells; and
(ii) T cells expressing an antibody-coupled T cell receptor (ACTR), wherein the ACTR comprises the amino acid sequence of SEQ ID NO:9.

79-97. (canceled)


SEQUENCE LISTING (40 sequences)

SEQ ID NO: 1 - protein, 442 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCQRRKYRSNKGESPVEPAEPCHYSCPREEEGSTIPIQEDYRKPEPACSPRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 2 - protein, 435 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 3 - protein, 428 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKKKYSSSVHDPNGEYMFMRAVNTAKKSRLTDVTLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 4 - protein, 431 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 5 - protein, 483 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSQRRKYRSNKGESPVEPAEPCHYSCPREEEGSTIPIQEDYRKPEPACSPRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 6 - protein, 469 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSKKKYSSSVHDPNGEYMFMRAVNTAKKSRLTDVTLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 7 - protein, 472 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 8 - protein, 477 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 9 - protein, 432 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - protein, 711 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 11 - protein, 391 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 12 - protein, 397 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQIYIWAPLAGTCGVLLLSLVITLYCQRRKYRSNKGESPVEPAEPCHYSCPREEEGSTIPIQEDYRKPEPACSPRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 13 - protein, 390 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 383 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQIYIWAPLAGTCGVLLLSLVITLYCKKKYSSSVHDPNGEYMFMRAVNTAKKSRLTDVTLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 386 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - protein, 395 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 17 - protein, 394 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 18 - protein, 666 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 19 - protein, 438 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 20 - protein, 419 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 21 - protein, 409 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 393 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 23 - protein, 19 aa, Artificial Sequence
  Synthetic polypeptide
ATNFSLLKQAGDVEENPGP

SEQ ID NO: 24 - protein, 254 aa, Artificial Sequence
  Synthetic polypeptide
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 25 - protein, 300 aa, Artificial Sequence
  Synthetic polypeptide
  MISC_FEATURE (16)..(300): may be absent
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 26 - protein, 436 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 27 - protein, 429 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQIEVMYPPPYLDNEKSNGTIIHVKGKHLCPSPLFPGPSKPIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 28 - protein, 15 aa, Artificial Sequence
  Synthetic polypeptide
GGGGSGGGGSGGGGS

SEQ ID NO: 29 - protein, 30 aa, Artificial Sequence
  Synthetic polypeptide
GGGGSGGGGSGGGGSGGGGSGGGGSGGGGS

SEQ ID NO: 30 - protein, 45 aa, Artificial Sequence
  Synthetic polypeptide
GGGGSGGGGSGGGGSGGGGSGGGGSGGGGSGGGGSGGGGSGGGGS

SEQ ID NO: 31 - protein, 60 aa, Artificial Sequence
  Synthetic polypeptide
GGGGSGGGGSGGGGSGGGGSGGGGSGGGGSGGGGSGGGGSGGGGSGGGGSGGGGSGGGGS

SEQ ID NO: 32 - protein, 75 aa, Artificial Sequence
  Synthetic polypeptide
GGGGSGGGGSGGGGSGGGGSGGGGSGGGGSGGGGSGGGGSGGGGSGGGGSGGGGSGGGGSGGGGSGGGGSGGGGS

SEQ ID NO: 33 - protein, 150 aa, Artificial Sequence
  Synthetic polypeptide
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 34 - protein, 225 aa, Artificial Sequence
  Synthetic polypeptide
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 35 - protein, 300 aa, Artificial Sequence; identical to SEQ ID NO: 25
  Synthetic polypeptide

SEQ ID NO: 36 - protein, 208 aa, Artificial Sequence
  Synthetic polypeptide
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQ

SEQ ID NO: 37 - protein, 254 aa, Artificial Sequence
  Synthetic polypeptide
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQVSFCLVMVLLFAVDTGLYFSVKTNIRSSTRDWKDHKFKWRKDPQDK

SEQ ID NO: 38 - protein, 411 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPG

SEQ ID NO: 39 - protein, 255 aa, Artificial Sequence
  Synthetic polypeptide
PMEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 40 - protein, 456 aa, Artificial Sequence
  Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPG